(19) United States
(12) Patent Application Publication
(10) Pub. No.: US 2022/0098619 A1
(43) Pub. Date: Mar. 31, 2022

(54) ANTI-CRISPR-MEDIATED CONTROL OF GENOME EDITING AND SYNTHETIC CIRCUITS IN EUKARYOTIC CELLS

(71) Applicant: The Board of Trustees of the Leland Stanford Junior University, Stanford, CA (US)

(72) Inventors: Muneaki NAKAMURA, Redwood City, CA (US); Lei S. Ql, Palo Alto, CA (US)

(21) Appl. No.: 17/312,324

(22) PCT Filed: Dec. 10, 2019
(86) PCT No.: PCT/US2019/065486

§ 371 Date: Jun. 9, 2021

Related U.S. Application Data
(60) Provisional application No. 62/777,652, filed on Dec. 10, 2018.

(51) Int. Cl. C12N 15/90; C12N 9/22; C07K 14/005; C12N 15/11; A61K 38/00

(52) U.S. Cl. CPC C12N 15/907; C12N 9/22; C07K 14/005; C12N 2795/00022; C12N 2310/20; C12N 2800/80; A61K 38/00; C12N 15/11

(57) ABSTRACT

Provided herein are, inter alia, compositions, methods, and systems for selectively modulating an activity of CRISPR-based systems, including CRISPR-based genome editing systems and CRISPR-based gene regulation in a target cell. Also provided are compositions, methods, and systems for controlling a gene regulation circuit or building dynamic pulsatile gene regulation circuits in a target cell, e.g., a mammalian cell by using CRISPR-based genome editing tools.

### CROSS-REFERENCE TO RELATED APPLICATIONS

This application claims the benefit of priority to U.S. Provisional Patent Application Ser. No. 62/777,652, filed on Dec. 10, 2018 which is herein expressly incorporated by reference it its entirety, including any drawings.

### INCORPORATION OF THE SEQUENCE LISTING

The material in the accompanying Sequence Listing is hereby incorporated by reference into this application. The accompanying Sequence Listing text file, named 078430-509001WO_Sequence Listing.txt, was created on Nov. 26, 2019 and is 16 KB.

### FIELD

The present disclosure provides compositions, methods, and systems for selectively modulating an activity of CRISPR-based systems, including CRISPR-based genome editing systems and CRISPR-based gene regulation systems in a target cell. The disclosure also provides compositions and methods for controlling gene regulation circuits or for building dynamic pulsatile gene regulation circuits in a target cell by using CRISPR-based genome editing tools.

### BACKGROUND

CRISPR (clustered regularly interspaced short palindromic repeats) systems originate from bacteria where they function as adaptive immune systems that cleave foreign nucleic acids in a sequence specific manner. CRISPR systems have been widely repurposed for biotechnological applications, including genome editing and gene expression regulation in prokaryotic and eukaryotic organisms. For example, the development of Type II CRISPR-Cas9 systems as programmable nucleases for genome engineering has been demonstrated beneficial in the field of biomedical sciences. In particular, various Cas9-based platforms have enabled gene editing in a large variety of biological systems, where both gene knockouts and tailor-made alterations are possible within complex genomes with good accuracy and efficiency. As such, CRISPR systems have the potential for application to gene therapy approaches for disease treatment, whether for the creation of custom, genome-edited cell-based therapies or for direct correction or ablation of aberrant genomic loci within human subjects. In addition, mutant versions of Cas9 in which the DNA nuclease activity has been inactivated (dCas9) have been developed for RNA-guided genome binding, enabling further applications in gene expression control and genome structure visualization.

While these technological advances bring potential benefits to medicine, agriculture, and the environment, concomitant challenges arise over the largely irreversible outcomes generated by genome editing, which in turn hamper the straightforward application of CRISPR-Cas systems for basic or applied research especially in multi-cellular systems. Means to safely and reversibly control the activity of CRISPR tools can mitigate security concerns related to their accidental or intentional misuse. However, the tools available to effectively counteract genome editing or gene regulation remain limited. For example, one major challenge is the insufficient specificity of the Cas nuclease and hence the risk of unintended editing of non-target loci. This issue was attempted to be resolved by employing engineered Cas variants with an improved on-target specificity or by timely activating/de-activating the Cas nuclease. Another major challenge is the need for highly specific delivery of CRISPR-Cas into and/or its activation within the desired target tissue or cell, ideally even after systemic administration of the CRISPR-Cas components. Therefore, the risk of undesired CRISPR-Cas activation in off-target tissues and, consequently, of undesired side-effects during an in vivo CRISPR-Cas application including gene therapy in humans remains a major concern. Furthermore, the safe application of CRISPR-Cas systems in gene therapy requires an ability to control the gene editing activity of a Cas/sgRNA complex once the intended use has been realized. While a number of engineered systems allow for controlled activation of CRISPR-Cas to increase precision, these systems still lack the ability to provide predictable control and robust inhibition.

Synthetic circuits controlling gene expression are another area of active interest for programming novel cellular behaviors, such as controlling development or implementing biological sensors and devices. Recently, interest in synthetic circuits implemented by CRISPR systems has increased due to the adaptability of CRISPR-based gene regulation. The complexity of implementable circuits is limited by the types of control nodes that can be wired to control CRISPR systems. To this end, methods of exogenous control over CRISPR systems have been developed, but internally programmed methods of control remain sparse.

What is needed in the art is the ability to predictably control gene editing activity or genome binding activity (e.g., using dCas9) to prevent unintended CRISPR-Cas cleavage or DNA binding activity once a specific goal has been attained. Additionally, the ability to restrict Cas cleavage activity to a particular site, tissue, or cell cycle stage would greatly improve the efficacy and safety of CRISPR-based clinical treatments and research applications.

Hence, additional tools and layers for controlling CRISPR systems activity are highly desired and urgently needed in order to reduce potential risks of off-target genome editing or gene regulation.

### SUMMARY

Provided herein, inter alia, are compositions and methods for selectively modulating the activity of a CRISPR system in various cells, e.g., human cells. In particular, certain aspects and embodiments of the disclosure provide various cells engineered to include an anti-CRISPR molecule which modulates an activity of a CRISPR system in the cells. Also provided are compositions and methods for inhibiting the activity of a CRISPR-based gene regulation system such as a CRISPR-based gene activation (CRISPRa) system or a CRISPR-based gene interference (CRISPRi) system. Also provided are compositions and methods for inhibiting the activity of a CRISPR-based genome editing system in a cell. Further provided are compositions and methods for treating a subject having or suspected of having a disorder or health condition employing ex vivo and/or in vivo genome editing. As described in greater detail herein, certain compositions and methods of the disclosure can be used for expanded applications to counteract CRISPR-mediated gene activation and repression of reporter and endogenous genes in various cell types.

In one aspect, provided herein are various engineered cells including: (a) one or more components of a CRISPR system including (i) a Cas endonuclease or a first nucleic acid encoding the Cas endonuclease, and (ii) at least one guide RNA (gRNA); and (b) an anti-CRISPR (Acr) polypeptide or a second nucleic acid encoding the Acr polypeptide, wherein the Acr polypeptide modulates an activity of the CRISPR system in an inducible and/or programmable manner.

Non-limiting exemplary embodiments of the engineered cells according to the present disclosure include one or more of the following features. In some embodiments, the at least one gRNA is encoded by a sequence incorporated in the first nucleic acid or in the second nucleic acid. In some embodiments, the CRISPR system further including a donor template that includes a sequence encoding a gene-of-interest (GOI). In some embodiments, the CRISPR system includes a Class 2 Cas endonuclease or a derivative thereof. In some embodiments, the Class 2 Cas endonuclease is a Type II Cas endonuclease or a Type V Cas endonuclease, or a derivative of any thereof.

In some embodiments, the Type II is a Cas9 endonuclease selected from the group consisting of a Streptococcus pyogenes Cas9, a Streptococcus thermophiles Cas9, a Staphylococcus aureus Cas9, a Brackiella oedipodis Cas9, a Neisseria meningitidis Cas9, a Haemophilus influenzae Cas9, a Simonsiella muelleri Cas9, a Ralstonia solanacearum Cas9, a Francisella novicida Cas9, and a Listeria monocytogenes Cas9, or a derivative of any thereof. In some embodiments, the Cas9 derivative is a nuclease-deficient Cas9 polypeptide (dCas9).

CRISPR system comprises a Type V Cas endonuclease or a derivative thereof. In some embodiments, the Type V Cas endonuclease is a Cas12 endonuclease selected from the group consisting of a Francisella novicida Cas12, a Lachnospiraceae bacterium ND2006 Cas12, and an Acidaminococcus sp. BV3L6 Cas12a, or a derivative thereof. In some embodiments, the Cas12 derivative is a nuclease-deficient Cas12 polypeptide (dCas12).

In some embodiments, the Cas endonuclease is operably linked to an effector domain is selected from the group consisting of an activation domain, a repression domain, a protein modification domain, a histone modification domain, a DNA modification domain, a RNA modification domain, and a heterodimerization domain.

In some embodiments, the CRISPR system is a CRISPR-based gene regulation system. In some embodiments, the CRISPR-based gene regulation system is a CRISPR-based gene activation (CRISPRa) system. In some embodiments, the CRISPRa system includes a dCas9 polypeptide or a Cas12 polypeptide operably linked to a transcriptional activation domain. In some embodiments, the transcriptional activation domain is selected from the group consisting of a VP16 activation domain, a VP64 activation domain, a p65 activation domain, a MyoD1 activation domain, a HSF1 activation domain, a RTA activation domain, a SETT/9 activation domain, a VP64-p65-Rta (VPR) activation domain, a mini VPR activation domain, a yeast GAL4 activation domain, a yeast HAP1 activation domain, and a histone acetyltransferase.

In some embodiments, the CRISPR-based gene regulation system is a CRISPR-based gene interference (CRISPRi) system. In some embodiments, the CRISPRi system includes a dCas9 polypeptide or a dCas12 polypeptide operably linked to a repression domain. In some embodiments, the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3A repressor domain, and a DNMT3L repressor domain.

In some embodiments, the CRISPR system is a CRISPR-based genome editing system. In some embodiments, the activity of the CRISPR-based genome editing system is selected from the group consisting of target polynucleotide binding, target polynucleotide double-strand break creation, target polynucleotide nicking (single-strand DNA cleavage), and target polynucleotide modification. In some embodiments, the target polynucleotide modification is selected from the group consisting of insertion of at least one nucleotide, deletion of at least one nucleotide, substitution of at least one nucleotide, and chemical alteration of at least one nucleotide.

In some embodiments, the Acr polypeptide includes a bacteriophage-derived Acr polypeptide. In some embodiments, the bacteriophage-derived Acr polypeptide is selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof. In some embodiments, the functional variants have at least 80% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, and AcrVA5.

In some embodiments, the expression and/or activity of the Acr polypeptide and/or the CRISPR system is inducible or programmable. In some embodiments, the Acr polypeptide and/or the Cas endonuclease is operably linked to an inducible destabilization domain (DD). In some embodiments, the inducible DD is selected from the group consisting of a DD from the rapamycin-binding protein (FKBP12), a DD from dihydrofolate reductase (DHFR), a DD from estrogen receptor ligand binding domain (ERLBD), and a DD from estrogen receptor (ER50). In some embodiments, the DD is from the FKBP12 and the stabilization of the DD is induced by addition of rapamycin analogue compound Shield1. In some embodiments, the Acr polypeptide is operably linked to a proteolytic cleavage site. In some embodiments, the proteolytic cleavage site can be cleaved by a protease selected from the group consisting of a tobacco etch virus (TEV) protease, a porcine teschovirus-1 2A (P2A) protease, a foot-and-mouth disease virus (FMDV) 2A (F2A) protease, an Equine Rhinitis A Virus (ERAV) 2A (E2A) protease, a Thosea asigna virus 2A (T2A) protease, a cytoplasmic polyhedrosis virus 2a (BmCPV2A) protease, a Flacherie Virus 2A (BmIFV2A) protease, thrombin, PreScission™ protease, a glutamyl endopeptidase, an Epstein-Barr virus protease, a matrix metalloproteinase 2 (MMP-2), a matrix metalloproteinase 1 (MMP-1), a membrane type 1 matrixmetalloproteinase (MT-MMP), a stromelysin 3 (or MMP-11), a matrix metalloproteinase 13 (collagenase-3), an MMP-3 (stromelysin), an MMP-7 (matrilysin), MMP-9, an NS3 protease, and a thermolysin-like MMP.

In some embodiments, the Acr polypeptide is further linked to one or more of the following: a nuclear localization signal (NLS), a G-protein-coupled receptor (GPCR), a Gly-Ser linker, and a synthetic Notch receptor.

In some embodiments, the Acr polypeptide and/or the one or more components of the CRISPR system are expressed episomally in the cell. In some embodiments, the Acr polypeptide and/or the one or more components of the CRISPR system are expressed from nucleic acids stably integrated in the genome of the cell.

In some embodiments, the expression of the Acr polypeptide and/or the one or more components of the CRISPR system is independently under control of a constitutive promoter, a repressible promoter, or an inducible promoter. In some embodiments, the constitutive promoter is selected from the group consisting of a SV40 promoter, a CMV promoter, a PGK promoter, a ubiquitin C (UBC) promoter, an EF1A promoter, a CAGG promoter, and a SFFV promoter.

In some embodiments, the inducible promoter is selected from the group consisting of a TRE3G inducible promoter, a tetracycline-regulated promoter, a steroid-regulated promoter, a metal-regulated promoter, an estrogen receptor-regulated promoter, and a UAS inducible promoter.

In some embodiments, the expression of the Acr polypeptide and/or the one or more components of the CRISPR system is under control of the same promoter.

In some embodiments, the engineered cell of the disclosure includes: (a) a first nucleic acid molecule encoding (i) an Acr polypeptide and (ii) a single guide RNA (sgRNA); and (b) a second nucleic acid molecule encoding a nuclease-deficient Cas polypeptide (dCas) fused to an effector domain, wherein the Acr polypeptide inhibits the activity of the dCas-effector domain fusion and wherein the expression of the Acr polypeptide and the CRISPRa system is under control of the same promoter. In some embodiments, the dCas polypeptideis a dCas9 polypeptideor a dCas12 polypeptide. In some embodiments, the first nucleic acid molecule is stably integrated into the genome of the cell. In some embodiments, the first nucleic acid molecule is maintained episomally in the cell.

In some embodiments, the cell is selected from the group consisting of a microbial cell, a fungal cell, a plant cell, and an animal cell, mammalian cell. In some embodiments, the animal cell is a mammalian cell. In some embodiments, the mammalian cell is a human cell. In some embodiments, the human cell is a stem cell. In some embodiments, the stem cell is a human-induced pluripotent stem cell (hiPSC).

In one aspect, provided herein are various kits including: (a) one or more components of a CRISPR system comprising (i) a Cas endonuclease or a first nucleic acid encoding the Cas endonuclease, and (ii) at least one gRNA; and (b) an anti-CRISPR (Acr) polypeptide or a second nucleic acid encoding the Acr polypeptide, wherein the Acr polypeptide modulates an activity of the CRISPR system in an inducible and/or programmable manner.

Non-limiting exemplary embodiments of the kits according to the present disclosure include one or more of the following features. In some embodiments, the kit further includes a sequence encoding a donor template encoding a gene-of-interest (GOI). In some embodiments, the sequence encoding the at least one gRNA and/or the sequence encoding a donor template is incorporated in the first nucleic acid. In some embodiments, the sequence encoding the gRNA and/or the sequence encoding a donor template is incorporated in the second nucleic acid.

In one aspect, provided herein are various methods for selectively modulating an activity of a CRISPR system in a cell. The methods include providing to the cell: (a) one or more components of a CRISPR system including (i) a Cas endonuclease or a first nucleic acid encoding the Cas endonuclease, and (ii) at least one gRNA; and (b) an anti-CRISPR (Acr) polypeptide or a second nucleic acid encoding the Acr polypeptide, wherein the Acr polypeptide modulates an activity of the CRISPR system in an inducible and/or programmable manner.

Non-limiting exemplary embodiments of the methods for selectively modulating an activity of a CRISPR system according to the present disclosure include one or more of the following features. In some embodiments, the at least one gRNA is encoded by a sequence incorporated in the first nucleic acid or in the second nucleic acid. In some embodiments, the CRISPR system further including a donor template that includes a sequence encoding a gene-of-interest (GOI).

In some embodiments, the CRISPR system includes a Class 2 Cas endonuclease or a derivative thereof. In some embodiments, the CRISPR system includes a Type II Cas endonuclease or a derivative thereof. In some embodiments, the CRISPR system includes a Type IIA Cas endonuclease or a derivative thereof. In some embodiments, the Type II Cas endonuclease is a Cas9 endonuclease selected from the group consisting of a Streptococcus pyogenes Cas9, a Streptococcus thermophiles Cas9, a Staphylococcus aureus Cas9, a Brackiella oedipodis Cas9, a Neisseria meningitidis Cas9, a Haemophilus influenzae Cas9, a Simonsiella muelleri Cas9, a Ralstonia solanacearum Cas9, a Francisella novicida Cas9, and a Listeria monocytogenes Cas9, or a derivative of any thereof. In some embodiments, the Cas9 derivative is a nuclease-deficient Cas9 polypeptide (dCas9).

In some embodiments, the CRISPR system includes a Type V Cas endonuclease or a derivative thereof. In some embodiments, the Type V Cas endonuclease is a Cas12 endonuclease selected from the group consisting of Francisella novicida Cas12, a Lachnospiraceae bacterium ND2006 Cas12, and an Acidaminococcus sp. BV3L6 Cas12a, or a derivative thereof. In some embodiments, the Cas12 derivative is a nuclease-deficient Cas12 polypeptide (dCas12).

In some embodiments, the nuclease-deficient Cas polypeptide is operably linked to an effector domain selected from the group consisting of an activation domain, a repression domain, a protein modification domain, a histone modification domain, a DNA modification domain, a RNA modification domain, and a heterodimerization domain.

In some embodiments, the CRISPR system is a CRISPR-based gene regulation system. In some embodiments, the CRISPR-based gene regulation system is a CRISPR-based gene activation (CRISPRa) system. In some embodiments, the CRISPRa system includes a dCas9 polypeptide or a dCas12 polypeptide operably linked to a transcriptional activation domain. In some embodiments, the transcriptional activation domain is selected from the group consisting of a VP16 activation domain, a VP64 activation domain, a p65 activation domain, a MyoD1 activation domain, a HSF1 activation domain, a RTA activation domain, a SETT/9 activation domain, a VP64-p65-Rta (VPR) activation domain, a mini VPR activation domain, a yeast GAL4 activation domain, a yeast HAP1 activation domain, and a histone acetyltransferase.

In some embodiments, the CRISPR-based gene regulation system is a CRISPR-based gene interference (CRISPRi) system. In some embodiments, the CRISPRi system includes a dCas9 polypeptide or a dCas12 polypeptide operably linked to a repression domain. In some embodiments, the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3A repressor domain, and a DNMT3L repressor domain.

In some embodiments, the CRISPR-based gene regulation system is a CRISPR-based genome editing system. In some embodiments, the activity of the CRISPR-based genome editing system is selected from the group consisting of target polynucleotide binding, target polynucleotide double-strand break creation, target polynucleotide nicking (single-strand DNA cleavage), and target polynucleotide modification. In some embodiments, the target polynucleotide modification is selected from the group consisting of insertion of at least one nucleotide, deletion of at least one nucleotide, substitution of at least one nucleotide, and chemical alteration of at least one nucleotide.

In some embodiments, the Acr polypeptide includes a bacteriophage-derived Acr polypeptide. In some embodiments, the bacteriophage-derived Acr polypeptide is selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof. In some embodiments, the bacteriophage-derived Acr polypeptide is AcrIIA4 or a derivative thereof. In some embodiments, the functional variants have at least 80% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, and AcrVA5. In some embodiments, the functional variants have at least 80% sequence identity to AcrIIA4. In some embodiments, the expression and/or activity of the Acr polypeptide and/or the CRISPR system is inducible or programmable

In some embodiments, the Acr polypeptide is provided by introducing into the cell a second nucleic acid molecule encoding the Acr polypeptide. In some embodiments, the second nucleic acid encoding the Acr polypeptide is introduced into the cell via transient delivery. In some embodiments, the Acr polypeptide is provided to the cell via stable integration of the second nucleic acid molecule into the genome of the cell.

In some embodiments, the Acr polypeptide and/or the Cas endonuclease is operably linked to an inducible DD. In some embodiments, the inducible DD is selected from the group consisting of a DD from the rapamycin-binding protein (FKBP12), a DD from dihydrofolate reductase (DHFR), a DD from estrogen receptor ligand binding domain (ERLBD), and a DD from estrogen receptor (ER50). In some embodiments, the DD is from FKBP12 and the stabilization of the DD is induced by addition of the stabilizing ligand rapamycin analogue compound Shield1. In some embodiments, the Acr polypeptide is operably linked to a proteolytic cleavage site. In some embodiments, the proteolytic cleavage site can be cleaved by a protease selected from the group consisting of a tobacco etch virus (TEV) protease, a porcine teschovirus-1 2A (P2A) protease, a foot-and-mouth disease virus (FMDV) 2A (F2A) protease, an Equine Rhinitis A Virus (ERAV) 2A (E2A) protease, a Thosea asigna virus 2A (T2A) protease, a cytoplasmic polyhedrosis virus 2a (BmCPV2A) protease, a Flacherie Virus 2A (BmIFV2A) protease, thrombin, PreScission™ protease, a glutamyl endopeptidase, an Epstein-Barr virus protease, a matrix metalloproteinase 2 (MMP-2), a matrix metalloproteinase 1 (MMP-1), a membrane type 1 matrixmetalloproteinase (MT-MMP), a stromelysin 3 (or MMP-11), a matrix metalloproteinase 13 (collagenase-3), an MMP-3 (stromelysin), an MMP-7 (matrilysin), MMP-9, an NS3 protease, and a thermolysin-like MMP.

In some embodiments, the Acr polypeptide is further linked to one or more of the following: a nuclear localization signal (NLS), a G-protein-coupled receptor (GPCR), a Gly-Ser linker, and a synthetic Notch receptor. In some embodiments, the Acr polypeptide and/or the one or more components of the CRISPR system are expressed episomally in the cell. In some embodiments, the Acr polypeptide and/or the one or more components of the CRISPR system are expressed from nucleic acids stably integrated in the genome of the cell.

In some embodiments, the expression of the Acr polypeptide and/or the one or more components of the CRISPR system is independently under control of a constitutive promoter, a repressible promoter, or an inducible promoter. In some embodiments, the constitutive promoter is selected from the group consisting of a SV40 promoter, a CMV promoter, a PGK promoter, a ubiquitin C (UBC) promoter, an EF1A promoter, a CAGG promoter, and a SFFV promoter. In some embodiments, the repressible promoter is a cumate promoter. In some embodiments, the inducible promoter is selected from the group consisting of a TRE3G inducible promoter, a tetracycline-regulated promoter, a steroid-regulated promoter, a metal-regulated promoter, an estrogen receptor-regulated promoter, and a UAS inducible promoter. In some embodiments, the expression of the Acr polypeptide and/or the one or more components of the CRISPR system is under control of the same promoter.

In some embodiments, a method for selectively modulating an activity of a CRISPR system according to the present disclosure includes (a) introducing into the cell a first nucleic acid molecule encoding (i) an Acr polypeptide and (ii) a sgRNA to produce an engineered cell; and (b) transiently transfecting the engineered cell from (a) with a second nucleic acid molecule encoding a nuclease-deficient Cas polypeptide (dCas) fused to an activation domain; wherein the Acr polypeptide inhibits the activity of the dCas-transcription domain fusion and wherein the dCas-transcription domain fusion activates the expression of the Acr polypeptide to create an incoherent feedforward loop (IFFL) gene regulation circuit. In some embodiments, the first nucleic acid molecule is stably integrated into the genome of the cell. In some embodiments, the first nucleic acid molecule is maintained episomally in the cell.

In some embodiments of the disclosure, the cell is selected from the group consisting of a microbial cell, a fungal cell, a plant cell, and an animal cell, mammalian cell. In some embodiments, the animal cell is a mammalian cell. In some embodiments, the mammalian cell is a human cell. In some embodiments, the human cell is a stem cell. In some embodiments, the stem cell is a human-induced pluripotent stem cell (hiPSC).

In some embodiments of the methods disclosed herein, the Acr polypeptide is provided to the cell concurrently with the introduction of at least one component of the CRISPR-based gene regulation system. In some embodiments, the Acr polypeptide is provided to the cell sequentially in relation to the introduction of the CRISPR-based gene regulation system. In some embodiments, the Acr polypeptide is provided to the cell prior to the introduction of the CRISPR-based gene regulation system. In some embodiments, the one or more components of the CRISPR-based gene regulation system is introduced into the cell via delivery of nucleic acids encoding the one or more components or via delivery of purified ribonucleoprotein (RNP) complex.

In another aspect, provided herein is a method for making a genetic circuit, including (a) providing a plurality of first nucleic acid molecules each encoding a nuclease-deficient Cas polypeptide (dCas) fused to an effector domain; and (b) introducing each of the first nucleic acid molecules into a host cell comprising a second nucleic acid molecule to produce a plurality of engineered cells, wherein the second nucleic acid molecule encodes (i) an Acr polypeptide and (ii) a gRNA capable of directing the dCas-effector domain fusion to a target gene in the engineered cells, and wherein the Acr polypeptide simultaneously inhibits the activity of the dCas-effector domain fusions expressed from the plurality of first nucleic acid molecules. In some embodiments, the dCas polypeptideis a dCas9 polypeptide. In some embodiments, the dCas polypeptide is a dCas12 polypeptide. In some embodiments, the dCas endonuclease comprises two non-contiguous portions: an N-terminal portion (A) and a C-terminal portion (B) which, when combined, forms an active dCas-effector domain fusion. In some embodiments, the Acr polypeptide inhibits the activity of the active dCas-effector domain fusion when both non-contiguous portions of dCas-effector domain fusion are present in the cell. In some embodiments, the first nucleic acid molecule and/or the second nucleic acid molecule is stably integrated into the genome of the cell. In some embodiments, the first nucleic acid molecule and/or the second nucleic acid molecule is maintained episomally in the cell. In some embodiments, the target gene is an endogenous gene of the cell. In some embodiments, the target gene is an endogenous gene in the host cell. In some embodiments, the target gene is a report gene selected from the group consisting of green fluorescent protein (GFP) gene, blue fluorescent protein (BFP) gene, yellow fluorescent protein (YFP) gene, luciferase gene, and mCherry gene. In some embodiments, the effector domain is an activation domain. In some embodiments, the expression of the target gene is monitored by live-cell time-lapse microscopy. In a related aspect, provided herein is a genetic circuit that is produced by a method according to a method for making a genetic circuit as disclosed herein.

In another aspect, provided herein are various methods of treating a disorder or health condition in a subject, the method including providing to a cell in the subject: (a) an anti-CRISPR (Acr) polypeptide or nucleic acid encoding the Acr polypeptide; and (b) a CRISPR-based system or nucleic acid encoding the system, wherein the Acr polypeptide modulates an activity of the CRISPR-based system in said cell.

In another aspect, provided herein is a method for controlling CRISPR-driven genome editing in a mammalian cell, the method includes administering into said cell one or more anti-CRISPR molecules as described and illustrated herein.

In another aspect, provided herein is a method for controlling a gene regulation circuit in a mammalian cell, the method includes administering into said cell one or more anti-CRISPR molecules as described and illustrated herein. In yet another aspect, some embodiments of the disclosure relate to a method for building dynamic pulsatile gene regulation circuits in cells, the method includes administrating into said cell one or more anti-CRISPR molecules as described and illustrated herein. In another aspect, some embodiments of the disclosure relate to a method using Acr-dynamic gene circuits for one or more of the following: (1) fine-tuning endogenous gene expression level, gene expression period, and kinetics for cell reprogramming and/or differentiation, (2) drug administration via cell based therapy, and (3) metabolic engineering, the method includes administrating into said cell one or more anti-CRISPR molecules as described and illustrated herein. In some embodiments, the one or more anti-CRISPR molecules includes bacteriophage-derived anti-CRISPR proteins (Acrs). In some embodiments, the Acrs are independently selected from AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof. In some embodiments, the functional variants have at least 80% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof. In some embodiments, the administered one or more anti-CRISPR molecules confers a CRISPR-based gene interference (CRISPRi) in the cell. In some embodiments, the administered one or more anti-CRISPR molecules confers a CRISPR-based gene activation (CRISPRa) in the cell.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIGS. 1A-1E summarize the results of experiments performed to show that CRISPR-based gene regulation provides quantitative characterization of Acr activity. FIG. 1A is a schematic representation of gene regulation mediated by sgRNA-programmed binding of dCas9 fused with gene activation domain VPR (CRISPRa) and gene repression domain KRAB (CRISPRi). Acrs that inhibit binding of DNA prevents CRISPRa and CRISPRi. FIG. 1B shows box diagrams for experimental assay of Acr activity. Cell lines with integrated reporter for CRISPRa (top) and CRISPRi (bottom) were transiently transfected with plasmids encoding appropriate dCas9 effector, sgRNA, and Acr variants fused via P2A peptide to the reporter mCherry. FIGS. 1C-1D show representative raw fluorescence flow cytometry traces for CRISPRa (FIG. 1C) and CRISPRi (FIG. 1D) assays. Negative control (dCas9 effector+non-targeting [n.t.] sgRNA) and positive control (dCas9 effector+active sgRNA) conditions in absence of Acr are compared to conditions with AcrIIC3 or AcrIIA4 incorporated in active sgRNA plasmid. Dotted line indicates median value of non-targeting sgRNA negative control. FIG. 1E shows a summary comparison of CRISPRa and CRISPRi activity in presence and absence of active and null Acrs (n.s.: p>0.05; **: p<0.01; ***: p<0.001) for n=7 (CRISPRa) and n=3 (CRISPRi) experimental replicates.

FIGS. 2A-D summarize the results of experiments performed to demonstrate that Acr regulates control of endogenous gene expression in different cell types. FIG. 2A shows box diagram for endogenous gene regulation experiments in human embryonic kidney (HEK293T) cells. Cells with integrated doxycycline-inducible CRISPRa or CRISPRi dCas9 effectors were transfected with plasmid encoding sgRNA and Acr fused in varying configurations to the reporter BFP via 2A peptide. FIG. 2A depicts change in mean expression of CXCR4 for CRISPRa and CRISPRi experiments in presence and absence of various Acr constructs for n=3 experimental replicates. FIG. 2C shows box diagram of endogenous gene regulation experiments in hiPSC. Cells with integrated dCas9-VPR-GFP were lentivirally transduced with sgRNA plasmids containing or lacking Acr. FIG. 2D depicts change in mean expression of CXCR4 in hiPSC cells without (−dox) and with (+dox) dCas9-VPR and in presence and absence of Acr for n=2 experimental replicates for Acr constructs and n=1 experimental replicate for “no Acr” condition.

FIGS. 3A-3E summarize the results of experiments performed to illustrate activity of Acrs in yeast editing and gene regulation. FIG. 3A is a schematic description for editing experiments in yeast cells, where yeast cells were simultaneously transformed with a plasmid encoding Cas9 with sgRNA targeting the essential gene TRP1 (chromosome IV) and a KanMX selection marker along with a plasmid expressing Acr. FIG. 3B shows representative images of colony formation on G418 plates. “No Acr” condition had a plasmid containing a coding sequence for mCherry in the place of Acr. FIG. 3C shows arithmetic means of colony formation efficiency in presence of various Acrs for n=3 experimental replicates. Error bar indicates+s.e.m. FIG. 3D are schemes for gene regulation experiments in yeast cells. CRISPRa (left panel)—a yeast strain bearing stably integrated Venus reporter from a TET-inducible promoter, dCas9, and MCP-VP64 was transformed with a plasmid expressing sgRNA capable of co-localizing MCP-VP64 to the TETO promoter, as well as Acr; CRISPRi (right panel)—a yeast strain bearing constitutive mCherry reporter and dCas9 was transformed with plasmid expressing sgRNA targeting reporter promoter and Acr. FIG. 3E shows Acr-induced change in reporter expression for CRISPRa and CRISPRi experimental conditions described in (d) for n=3 experimental replicates.

FIGS. 4A-4D summarize the results of experiments performed to demonstrate Acr activity in inducible CRISPRa contexts. FIG. 4A is box diagram of GPCR activation experiment, where a reporter cell line was transiently transfected with plasmids encoding sgRNA, synthetic GPCR with TEV protease and Acr, and β-arrestin-2 fused to dCas9-VPR via a TEV cleavage site (TCS). FIG. 4B depicts change in reporter fluorescence with various Acr constructs in absence and presence of GPCR-activating ligand clozapine-N-oxide (CNO) for n=2 experimental replicates. FIG. 4C is a diagram of Shield1-inducible control experiment: a reporter cell line was transiently transfected with CRISPRa VPR-dCas9 plasmid along with sgRNA plasmid containing fusions of AcrIIA4 to Shield1-stabilized destabilization domain. FIG. 4D: Inducible control of gene activation from DD-AcrIIA4 fusions as a function of stabilization reagent Shield1 for n=4 experimental replicates. Shaded region indicates ±s.e.m.

FIGS. 5A-5C summarize the results of experiments performed to demonstrate that cells with integrated AcrIIA4 become “write-protected” against future editing. FIG. 5A: HEK293T cells were lentivirally transduced with a cassette constitutively expressing AcrIIA4. A clonal line was isolated and compared to untransduced wild-type cells. Cas9+sgRNA was delivered either by a plasmid expressing both components or purified ribonucleoprotein complex (RNP). FIG. 5B summarize the results from a T7E1 assay comparing editing efficiency between wild-type (WT) and AcrIIA4 (WPC) line targeting the PD1 locus run on an agarose gel with a 100 bp standard. Predicted lengths for uncut and cleaved products are annotated. FIG. 5C shows editing efficiency as quantified by TIDE analysis, with plasmid delivery, unsorted (plasmid) and sorted (sorted), as well as RNP delivery at various genomic loci. Error bars indicate s.e.m. of n=2 experimental replicates for plasmid delivery and an estimate of technical variance (s.d.) of a single experimental replicate for RNP delivery. The dotted line is an estimate of detection sensitivity computed from sequencing traces of unedited cells (mean+2×s.d.).

FIGS. 6A-6J summarize the results of experiments performed to illustrate an exemplary pre-programmed Acr-based genetic circuits according to some embodiments of the disclosure. FIG. 6A is a schematic representation of Genetic circuits analyzed via live-cell microscopy. CRISPRa: dCas9-VPR drives inducible GFP reporter expression; Acr: constitutive expression of AcrIIA4 prevents dCas9-VPR-based CRISPRa activity; IFFL: dCas9-VPR simultaneously drives inducible GFP and AcrIIA4 expression, resulting in a pulse of activity. FIG. 6B shows selected snapshots of cell-tracking traces of the IFFL circuit. Each row corresponds to a single trace, with time post-transfection annotated above each frame. FIGS. 6C-6J depict experimental and computational exploration of IFFL circuit activity. Circuit activity (y-axis) corresponds to GFP production shown in FIG. 6C and computed response for an arbitrary circuit output for FIGS. 6D-6J. FIG. 6C shows aligned activity of IFFL condition. Shown are the time-dependent median expression of GFP of cell tracking traces for two separate experiments. Overlaid are median fits (solid lines) and the combined density plots of both experiments (gray areas), encompassing n=187 cell traces. t=0 corresponds to aligned maximum of pulse for each trace. FIGS. 6D-6J show parameter sensitivity analysis from computational modeling. Plotted are predictions of behavior of the IFFL circuit when changing the following parameters: (1) dCas9-VPR production rate (FIG. 6D); (2) dCas9-VPR degradation rate (FIG. 6E); (3) dCas9-VPR-dependent output production rate (FIG. 6F); (4) output degradation rate (FIG. 6G); (5) dCas9-VPR-dependent Acr production rate FIG. 6H); (6) Acr degradation rate FIG. 6I); and (7) dCas9-Acr interaction rate FIG. 6J). The thick black line for each plot corresponds to computed activity for the value of that parameter derived from fits to experimental data, while other lines correspond to alterations to that parameter plus or minus one order of magnitude. All plots share axis units as annotated in (d). t=0 corresponds to activation of circuit response

FIGS. 7A-7C summarize the results of experiments performed to demonstrate that CRISPRa and CRISPRi reveal differences in Acr activity. FIG. 7A shows experimental schemes for assessing Acr variant activity for CRISPRa (left panel) and CRISPRi (right panel). Reporter cell lines were transfected with plasmids encoding sgRNA, dCas9-effector, and Acr. FIG. 7B shows representative raw flow cytometry traces of reporter fluorescence in presence of various Acrs. Dotted line indicates median fluorescence of untransfected condition. FIG. 7C is a summary of effect of panel of Acrs on CRISPRa (left) and CRISPRi (right). (*: p<0.05; **: p<0.01; ***: p<0.001) for n=5 (CRISPRa) and n=3 (CRISPRi) experimental replicates.

FIGS. 8A-8B summarize the results of experiments performed to compare activities of AcrIIA5 and AcrIIA6. Results from a dual-plasmid CRISPRa experiment comparing additional Acr families. Plasmid bearing sgRNA+Acr variant was co-transfected with dCas9-VPR plasmid. FIG. 8A shows raw flow cytometry traces of GFP fluorescence from a single experimental replicate. Dotted line indicates median fluorescence of untransfected condition. FIG. 8B depicts the mean fluorescence for each condition normalized to the negative control sgRNA condition.

FIGS. 9A-9C summarize the results of experiments performed to illustrate that AcrIIA4 regulates CRISPRa and CRISPRi on endogenous CXCR4 expression. FIGS. 9A-9B depict representative raw fluorescence flow cytometry traces of CXCR4 immunostaining for HEK293T CRISPRa (FIG. 9A) and HEK293T CRISPRi (FIG. 9B). Dotted line indicates median fluorescence of untransfected condition. FIG. 9C: CXCR4 CRISPRa in hiPSC. Traces compare conditions in absence (−dox) and presence (+dox) of dCas9-VPR effector. Dotted line indicates median of dCas9-VPR-only cell line (+dox).

FIGS. 10A-10B summarize the results of experiments performed to show that Acrs demonstrate varying activity depending on experimental context. FIG. 10A depicts toxicity of Acrs in yeast. Yeast were transformed with plasmids bearing Acr variants and grown under uracil auxotrophic conditions. AcrIIA3 demonstrates a clear toxic phenotype compared to other Acrs tested. FIG. 10B depicts representative raw flow cytometry density traces of reporter expression for CRISPRa and CRISPRi experiments in yeast.

FIG. 11A-11D summarize the results of experiments performed to demonstrate that Acr activity depends on stoichiometric ratio and fusion context. FIG. 11A is a schematic for free dCas9 controls: a reporter cell line is transiently transfected with a plasmid encoding sgRNA, synthetic GPCR with TEV protease, and Acr simultaneously with one encoding dCas9-VPR.

FIG. 11B is a summary of activity from n=2 experimental replicates of various Acr fusions under conditions from FIG. 11A. Fold changes in GFP expression are labeled for each condition. As shown in FIG. 11C, Acr:VPR-dCas9 expressed at roughly 1:1 ratio. Reporter cells are transiently transfected with sgRNA plasmid and a construct fusing Acr to VPR-dCas9 via a P2A self-cleaving linker. FIG. 11D depicts a comparison of Acr activity of constructs from the experiment described in FIG. 11C. Fold changes in GFP expression are labeled for each condition. n=1 experimental replicate for AcrIIC3 and n=2 for AcrIIA4 condition.

FIGS. 12A-12C summarize the results of experiments performed to demonstrate the effects of Shield1 ligand on CRISPRa, Acr, and DD-VPR-dCas9. As shown in FIG. 12A, control plasmids for CRISPRa, as well as CRISPRa with Acr lacking DD were transiently transfected into a reporter cell. In FIG. 12B, a DD-domain was fused to VPR-dCas9 and assessed for CRISPRa activity. FIG. 12C (left panel): Activity of CRISPRa with non-targeting guide (n.t. sgRNA), on-target guide (target sgRNA), on-target guide with AcrIIC3 (AcrIIC3) and on-target guide with AcrIIA4 in presence and absence of Shield1. FIG. 12C (right panel): Activity of CRISPRa with DD-VPR-dCas9 fusion across Shield1 concentrations. Shaded regions indicate ±s.e.m. n=4 for all conditions except DD-VPR-dCas9 at Shield1 concentrations above 0 nM (n=1)

FIGS. 13A-13B summarize the results of experiments performed to demonstrate that AcrIIA4 mediates editing efficiency in a mammalian reporter system. A cell line was generated with an integrated editing reporter: a split GFP with the complementary strand in an out-of-frame context. Indels induced by Cas9 editing allow for the formation of in-frame expression, resulting in gain of fluorescence in the edited cell population. FIG. 13A depicts flow cytometry traces demonstrating an increase in GFP expression upon genome editing, and diminished efficiency of editing in presence of AcrIIA4. FIG. 13B shows quantified proportion of GFP-positive cells in presence and absence of Acr for two experimental replicates.

FIGS. 14A-14D summarize the results of experiments performed to illustrate the gene regulation circuit performance as assessed by live-cell microscopy. FIG. 14A depicts an experimental scheme for live-cell microscopy assays. Reporter cells with inducible GFP were stably integrated with plasmid with sgRNA and constitutive expression of mCherry alone (CRISPRa condition), constitutive expression of mCherry+AcrIIA4 (Acr condition), or inducible expression of mCherry+AcrIIA4 (IFFL condition). The circuits were started by transient transfection of plasmid containing VPR-dCas9. FIG. 14B shows unaligned density plots (gray areas) of computationally detected cell traces across CRISPRa (left), Acr (center), and IFFL (right) conditions. Median fluorescence is plotted as discrete points. FIG. 14C depicts aligned density plots of two separate experiments for CRISPRa (left) and Acr (right) conditions. Median fluorescence is plotted as discrete points, and median cell response fit is plotted as solid lines. Number of cell traces is n=472 for CRISPRa and n=6 for Acr conditions (only 6/97 cells passed alignment step due to low overall fluorescence). FIG. 14D depicts aligned density plot and median fluorescence of transient transfection of both IFFL and VPR-dCas9 plasmids from n=8 cells (8/64 passed alignment step).

FIGS. 15A-15C summarize the results of experiments performed to illustrate the parameterization of model based on recapitulation of experimental circuit response, comparison of mean fluorescence data of experimental traces, and parameterized models. A more detailed description of the Supplementary Modeling is discussed in Example 2 below) for CRISPRa (FIG. 15A), Acr (FIG. 15B), and IFFL circuit conditions (FIG. 15C).

FIG. 16 schematically summarizes the results of experiments performed to demonstrate the degradation of a DD-Acr fusion polypeptide (ArcIIA4) in the presence of Shield1. In these experiments, a dCas9-miniVPR fusion was placed under control of PGK promoter (PGK-miniVPR) or EF1a promoter (EF1a-miniVPR). Two versions of ArcIIA4 were tested on inducible control of gene expression: (1) N-terminal DD-ArcIIA4; (2) C-terminal: AcrIIA4-DD; and (3) Acr only (no fusion; control), each of which was placed under control of three promoters (CMV-, PGK-, EF1a-) and configuration (N-terminal: DD-AcrIIA4; C-terminal: AcrIIA4-DD; no fusion: AcrIIA4). The (−) and (+) at the bottom of the graph each refer to absence or presence of stabilizing ligand Shield1, respectively. In this experiment, log-fold gene expression changes are plotted for n=1 experimental replicate.

FIG. 17 schematically summarizes the results of experiments performed to demonstrate the degradation of an exemplary DD-Cas9 polypeptide fused to an activation domain in the presence of Shield1. In these experiments, two fusions of DD and dCas9-miniVPR were tested on inducible gene regulation: (1) N-terminal: CMV-DD-dCas9-miniVPR; and (2) C-terminal: CMV-dCas9-miniVPR-DD. sgGAL4 condition indicates activity with non-targeting gRNA. sgTET indicates activity with targeting gRNA. The presence or absence of stabilizing ligand Shield1 is indicated by (−) and (+) on X-axis. Log-fold gene expression changes are plotted for a single experimental replicate.

FIG. 18 schematically summarizes the results of experiments performed to demonstrate genome editing efficiencies at two different loci. In these experiments, human iPSCs were transfected via editing Cas9 RNP targeting two genomic loci RELA and CDCl42BPB, as indicated on X-axis. For each locus, two cell lines were transfected: a basal wild-type line, and a write-protection line that was created by lentiviral transduction of a constitutive Acr cassette followed by sorting of positive cells. Editing efficiencies are shown as determined by TIDE analysis as plotted for n=2 experimental replicates. Dotted line indicates detection threshold as determined by TIDE analysis on untransfected cell.

FIG. 19 schematically summarizes the results of experiments performed to demonstrate a combinatorial logic over gene expression using Acrs. In these experiments, a dCas12a-miniVPR protein with a leucine zipper was split into two polypeptide portions: an N-terminal portion (A) and a C-terminal portion (B) which could spontaneously recombine into an active dCas12a-effector fusion protein when polypeptide portions are expressed. dCas12a-effector fusion activity is inhibited in the presence of a third polypeptide, i.e., AcrVA1 protein (C). Therefore, the three polypeptides constitute a logic gate wherein gene activation occurs only when A and B are present and C is absent (i.e., A AND B AND NOT C). Right panel: gene activation data confirming the activity of the performance of the logic gate.

### DETAILED DESCRIPTION OF THE DISCLOSURE

The present disclosure generally relates to, inter alia, compositions and methods for selectively modulating an activity of a CRISPR-based system, such as a CRISPR-based genome editing system or CRISPR-based gene regulation system in a target cell. Also provided are compositions, methods, and systems for controlling a gene regulation circuit or building dynamic pulsatile gene regulation circuits in a target cell, e.g., a mammalian cell by using CRISPR-based genome editing tools.

As discussed in greater detail below, repurposed CRISPR-Cas systems provide a useful tool set for broad applications of genomic editing and regulation of gene expression in prokaryotes and eukaryotes. Recent discovery of phage-derived proteins, anti-CRISPR (Acr) proteins, which serve to abrogate natural CRISPR anti-phage activity, potentially expands the ability to build synthetic CRISPR-mediated circuits. The experiments described in the Examples below were performed to characterize and evaluate of a panel of Arc molecules for expanded applications, including counteract CRISPR-mediated gene activation and repression of reporter genes and endogenous genes in various cell types. The results of certain experiments described herein demonstrated that cells pre-engineered with Arc molecules become resistant to genome editing, thus providing a means to generate “write-protected” cells that prevent future genome editing. The results of other experiments described herein also demonstrated that Arc proteins can be used to control CRISPR-based gene regulation circuits, including implementation of a pulse generator circuit in mammalian cells. The data described herein indicates that Arc proteins can serve as widely applicable tools for synthetic systems regulating the behavior of eukaryotic cell.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. As such, the term “about” is used to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “cell”, “cell culture”, “cell line”, “engineered cell”, “recipient cells” and “host cells” as used herein, include the primary subject cells and any progeny thereof, without regard to the number of transfers. It should be understood that not all progeny are exactly identical to the parental cell (due to deliberate or inadvertent mutations or differences in environment); however, such altered progeny are included in these terms, so long as the progeny retain the same functionality as that of the originally transformed cell.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA.” The spacers are short segments of DNA from a virus and may serve as a “memory” of past exposures to facilitate an adaptive defense against future invasions.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective alteration of a specific genomic target. Such a specific genomic target includes, but is not limited to, a chromosomal region, a gene, a promoter, an open reading frame or any nucleic acid sequence.

The term “engineered” or “recombinant” when used with reference to a cell, a nucleic acid, a protein, or a vector, indicates that the cell, nucleic acid, protein or vector has been altered through human intervention such as, for example, has been modified by or is the result of laboratory methods. Thus, for example, recombinant or engineered proteins and nucleic acids include proteins and nucleic acids produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence. The term “engineered” when used in reference to a cell is not intended to include naturally-occurring cells but encompass cells that have been modified to include or express a polypeptide or nucleic acid that would not be present in the cell if it was not engineered.

As used herein, the term “variant” of a polypeptide (e.g., Acr polypeptide) refers to a polypeptide in which one or more amino acid substitutions, deletions, and/or insertions are present as compared to the amino acid sequence of a reference Acr polypeptide. The term “functional variant thereof” relates to a molecule having quantitative and qualitative biological activity in common with the wild-type molecule from which the fragment or variant was derived. For example, when referencing a polypeptide having an enzymatic activity (e.g., an enzyme such as a Cas endonuclease, the term “functional variant” refers to an enzyme that has a polypeptide sequence that is at least about 70%, at least about 75%, at least about 80%, at least about 85%, at least about 90%, at least about 95% or at least about 99% identical to a polypeptide sequence encoding the enzyme. The “functional variant” enzyme may retain amino acids residues that are recognized as conserved for the enzyme, and may have non-conserved amino acid residues substituted or found to be of a different amino acid, or amino acid(s) inserted or deleted, but which does not affect or has insignificant effect its enzymatic activity, as compared to the enzyme described herein. The “functional variant” enzyme has an enzymatic activity that is identical or essentially identical to the biological activity of the enzyme (e.g., Cas) described herein. One skilled in the art will appreciate that the “functional variant” polypeptide may be found in nature, e.g., naturally occurring, or be an engineered mutant thereof.

The term “modulating” refers to decreasing, reducing, inhibiting, increasing, inducing, activating, or otherwise affecting the expression or activity of a polypeptide. The term may also refer to decreasing, reducing, inhibiting, increasing, inducing, activating, or otherwise affecting the activity of a gene encoding a polypeptide which can include, but is not limited to, modulating transcriptional activity.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

The term “operably linked”, as used herein, denotes a physical or functional linkage between two or more elements, e.g., polypeptide sequences or polynucleotide sequences, which permits them to operate in their intended fashion.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity typically is calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, the term “constitutive promoter” refers to promoters that are active in all circumstances in the cell. As used herein, the terms “inducible promoter” or “repressible promoter” refers to promoters that become active or repressed in response to specific stimuli.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### CRISPR Endonuclease Systems

CRISPR (Clustered, Regularly Interspaced Short Palindromic Repeats) systems can be found in the genomes of many prokaryotes (e.g., bacteria and archaea) and mediate specific degradation of foreign, invading nucleic acids. They comprise a CRISPR-associated (Cas) nuclease which can be programmed by short guide RNAs (gRNAs) to induce double-strand breaks at specific, sequence-complementary DNA loci. Cas nucleases are also referred to as “programmable nuclease” in that they can be “targeted” (“programed”) to recognize and edit a pre-determined genomic location. In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid.

CRISPR-Cas systems belong to two classes, with multi-subunit effector complexes in Class 1 and single-protein effector modules in Class 2. It has been reported that Class 1 systems use a complex of multiple Cas proteins to degrade foreign nucleic acids. On the other hand, Class 2 systems use a single large Cas protein for the same purpose. Class 1 is divided into types I, III, and IV; class 2 is divided into types II, V, and VI. These six system types are divided into 19 subtypes. Class 1 Cas endonucleases encompasses that following subtypes: I, IA, IB, IC, ID, IE, IF, IU, III, IIIA, IIIB, IIIC, IIID, IV, IVA, and IVB. Class 2 Cas endonucleases encompasses that following subtypes: II, IIA, IIB, IIB, IIC, V, and IV. Each type and most subtypes are characterized by a “signature gene” found almost exclusively in the category. Classification is also based on the complement of cas genes that are present. Most CRISPR-Cas systems have a Casl protein. The phylogeny of Casl proteins generally agrees with the classification system. Many organisms contain multiple CRISPR-Cas systems suggesting that they are compatible and may share components. The sporadic distribution of the CRISPR/Cas subtypes suggests that the CRISPR/Cas system is subject to horizontal gene transfer during microbial evolution.

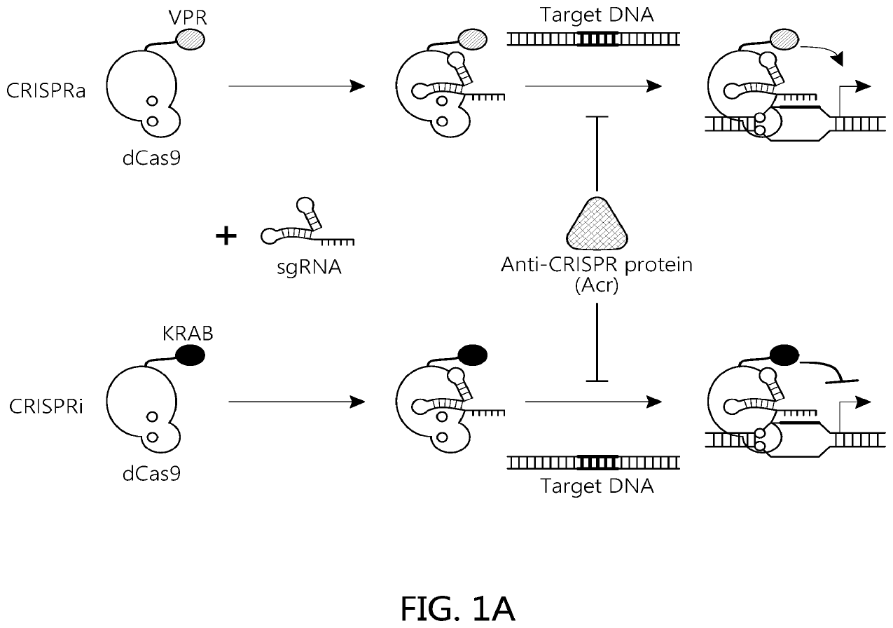

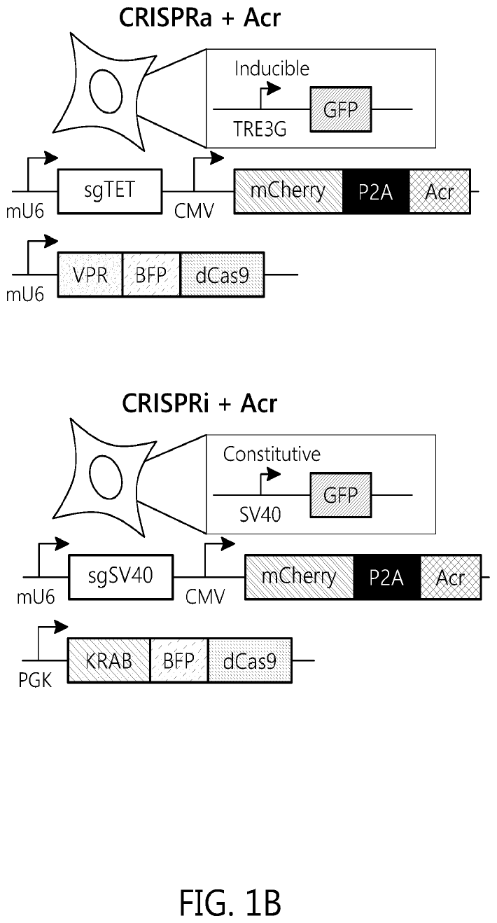

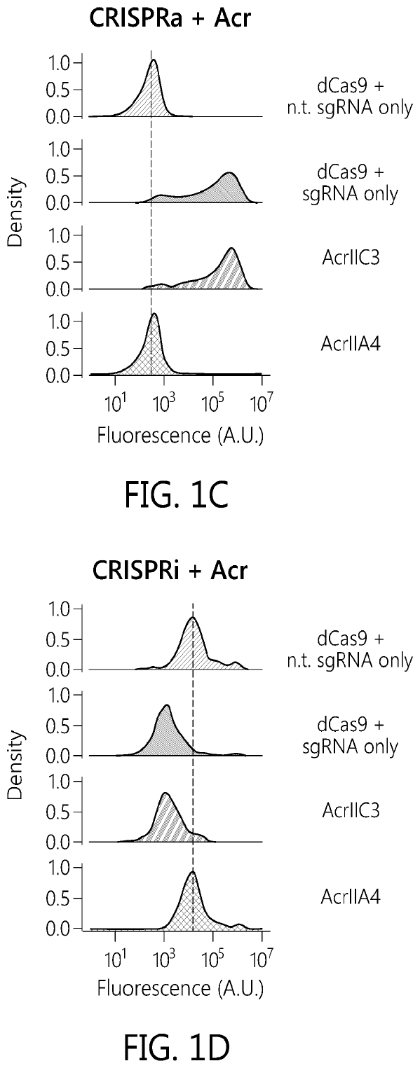

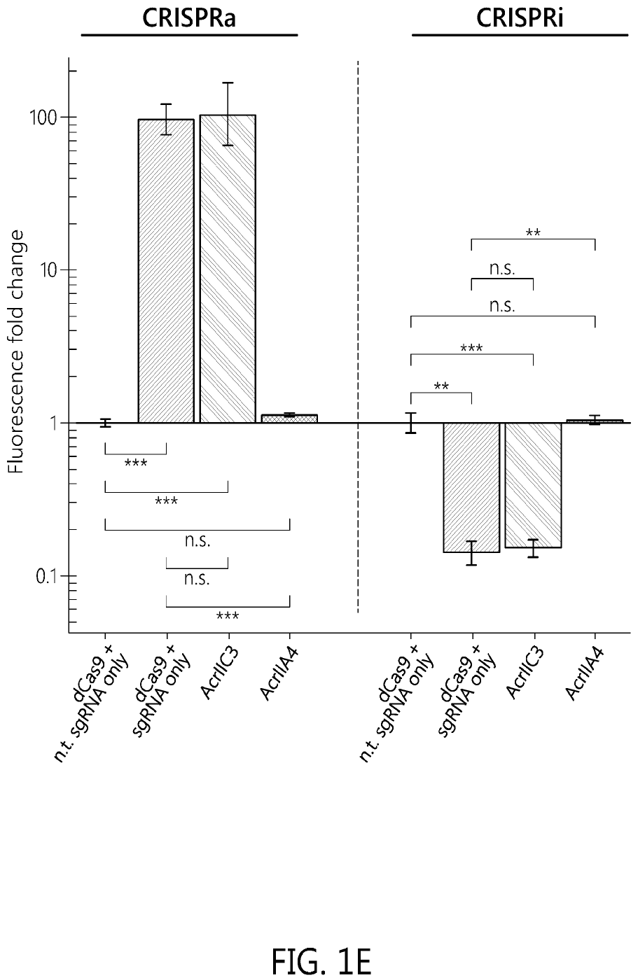

A CRISPR locus includes a number of short repeating sequences referred to as “repeats.” When expressed, the repeats can form secondary hairpin structures (e.g., hairpins) and/or have unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA. A CRISPR locus also has polynucleotide sequences encoding CRISPR-associated (Cas) genes. Cas genes encode endonucleases involved in the biogenesis and the interference stages of crRNA function in prokaryotes. The term “cas gene” (for CRISPR-associated) has its conventional meaning as used in the art where it refers to a gene that is coupled to, associated with, close to, or in the vicinity of a CRISPR array. As such, “cas gene” includes, but is not limited to, cas, csn, csm and cmr genes, depending upon the type of CRISPR-Cas system. A person skilled in the art can readily identify based on conventional protein comparison bioinformatics tools (such as BLAST), whether a gene associated with a CRISPR locus encodes a Cas polypeptide characteristic of any CRISPR-Cas system. The expression “Cas polypeptide” encompasses Cas, Csn, Csm and Cmr polypeptides, depending upon the type of CRISPR-Cas system. Exemplary CRISPR/Cas polypeptides include the Cas9 polypeptides in FIG. 1 of Fonfara et al., Nucleic Acids Research, 42: 2577-2590 (2014), which is herein incorporated by reference.

### Class 2 CRISPR Systems

As discussed above, Class 2 CRISPR-Cas systems refer to CRISPR-Cas systems functioning with a single protein as effector complex. crRNA biogenesis in a Class 2 CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed endonuclease (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Class 2 CRISPR system is referred to as a protospacer adjacent motif (PAM). In nature, the PAM is essential to facilitate binding of a site-directed endonuclease (e.g., Cas9) to the target nucleic acid. Class 2 Cas endonucleases encompasses that following subtypes: II, IIA, IIB, IIB, IIC, V, and IV. Each type and most subtypes are characterized by a “signature gene” found almost exclusively in the category. Type II CRISPR systems (also referred to as Nmeni or CASS4) are further subdivided into Type II (Cas9), Type IIA (CASS4), and Type IIB (CASS4a). Type II CRISPR-Cas system refers to CRISPR-Cas systems comprising cas9. Type IIA CRISPR-Cas system refers to CRISPR-Cas systems comprising csn2 genes. Type IIB CRISPR-Cas system refers to CRISPR-Cas systems comprising the cas4 genes. Type IIC CRISPR-Cas system refers to CRISPR-Cas systems comprising the cas9 gene but neither the csn2 nor the cas4 gene. Type V CRISPR-Cas system refers to CRISPR-Cas systems comprising the C2c1, C2c3, cas12 genes (cas12a, cas12b, or cas12c gene) in its cas genes. Type VI CRISPR-Cas system refers to CRISPR-Cas systems comprising the cas13 gene, such as cas13a (previously known as C2c2), cas13b, cas13c, and cas13d genes in its cas genes. The classification used for the distinction between Class 1 CRISPR-Cas systems and Class 2 CRISPR-Cas systems is often based on the genes encoding the effector molecules. Several recent studies demonstrated that the CRISPR/Cas9 system is useful for RNA-programmable genome editing, and provided numerous examples and applications of the CRISPR/Cas endonuclease system for site-specific gene editing. Additional information regarding the classification and evolution of Class 2 CRISPR-Cas systems can be found in, for example, a review by Koonin E. V. et al, (Current Opinion in Microbiology, Vol. 37, June 2017, pp. 67-78), which is herein incorporated by reference.

The CRISPR-Cas system uses a RNA molecule called a single guide RNA (sgRNA) that contains nucleotides of sequence complementary to the desired target site, and targets an associated Cas nuclease (for example the Cas9 nuclease) to a specific sequence in DNA. This targeting occurs by Watson-Crick based pairing between the sgRNA and the sequence of the genome within the approximately 20 bp targeting sequence of the sgRNA. Once bound at a target site, the Cas nuclease cleaves both strands of the genomic DNA creating a double strand break. The only requirement for designing a sgRNA to target a specific DNA sequence is that the target sequence must contain a protospacer adjacent motif (PAM) sequence at the 3′ end of the sgRNA sequence that is complementary to the genomic sequence. In the case of the Cas9 nuclease, the PAM sequence is NRG (where R is A or G and N is any base), or the more restricted PAM sequence NGG. PAM motifs occur on average very 15 bp in the genome of eukaryotes. Therefore, sgRNA molecules that target any region of the genome can be designed in silico by locating the 20 bp sequence adjacent to all PAM motifs. Because sgRNA can be rapidly synthesized in vitro, this enables the rapid screening of all potential sgRNA sequences in a given genomic region to identify the sgRNA that results in the most efficient cutting.

As discussed in some embodiments of the disclosure, a Cas derivative can be devoid of at least one of its activities, such as for example a Cas9 polypeptide or a Cas12 polypeptide devoid of its nuclease activity, which is also referred to as a nuclease-null Cas9 or nuclease-null Cas12, a catalytically-inactive Cas9 or catalytically-inactive Cas12, nuclease-deficient Cas9 polypeptide or nuclease-deficient Cas12 polypeptide, or “dCas9” or “dCas12.” In dCas9 polypeptide, the nuclease activity has been disabled by mutating residues in the RuvC and HNH catalytic domains. Disabling of both cleavage domains can convert Cas9 from a RNA-programmable nuclease into an RNA-programmable DNA recognition complex to deliver effector domains to specific target sequences.

A catalytically-inactive programmable RNA-dependent DNA-binding protein (e.g., dCas9) can be generated by mutating the endonuclease domains within Cas9, which can modulate transcription in prokaryotes or eukaryotes either directly or through an incorporated effector domain, such as a transcriptional activation domain (CRISPRa) or repressor domain (CRISPRi). As such, CRISPR-associated catalytically inactive dCas9 protein offers a general platform for RNA-guided DNA targeting. Fusion of dCas9 to effector domains with distinct regulatory functions enables stable and efficient transcriptional repression or activation in a target cell, with the site of delivery determined solely by a co-expressed sgRNA.

The experimental data presented herein demonstrate that Class 2 CRISPR-Cas systems can be engineered to enable targeted RNA-guided genome regulation in target cells by tethering transcriptional activation domains either directly to a catalytically inactive (nuclease-deficient) Cas9 polypeprtide (dCas9) or a catalytically inactive Cas12 polypeptide (dCas12) to a sgRNA.

### Anti-Crispr Polypeptides

As outlined above, various embodiments and aspects of the present disclosure include polypeptides that interfere with a function of a bacterial CRISPR-Cas system. Such polypeptides are referred herein as “anti-CRISPR proteins” (Acrs). The interference of an Arc polypeptide with a function of a bacterial CRISPR-Cas system is often defined as significantly down-modulates the activity of the CRISPR-Cas system, e.g., decreases, from partial to complete inhibition, the activity of the CRISPR-Cas system. Such down-modulation can be assayed by various methods known in the art. For example, the interference of an Arc polypeptide with a function of a bacterial CRISPR-Cas system can be assayed by providing a bacterial strain which is resistant to a given virulent phage, said resistance being mediated by a given CRISPR-Cas system (e.g., the CRISPR array of the given CRISPR-Cas system includes a spacer corresponding to a protospacer found in the genome of the virulent phage); introducing into bacterial strain an Arc polypeptide (interfering with a function of a bacterial CRISPR-Cas system); and exposing said bacterial strain to said given virulent phage, wherein an increase of the titer of said virulent phage is recorded as compared to a control.

Recently, the discovery of a set of bacteriophage-derived Arc polypeptides that inactivate certain CRISPR systems revealed the existence of an evolutionary arms race between these adaptive immune systems and infectious agents. These naturally occurring inhibitors can potentially inactivate CRISPR-Cas systems when needed, or decrease off-target effects, without significantly affecting on-target efficiency. Thus, these proteins can be used as a promising method to control CRISPR-Cas activity. Acrs were initially discovered for Type I CRISPR systems. It was not until fairly recently that Acrs targeting the Class 2 CRISPR systems, including the protein mostly widely used for genomic engineering, S. pyogenes (Spy) CRISPR-associated protein 9 (Cas9), were identified. Subsequent bioinformatics mining identified Acr activity against Type IIC Cas9 from Neisseria meningitidis (AcrIIC1, AcrIIC2, and AcrIIC3) and Type IIA Cas9 from Streptococcus pyogenes (spCas9) (AcrIIA1, AcrIIA2, AcrIIA3, and AcrIIA3). More recently an additional Acr against spCas9 was identified by cloning and testing multiple genes from a phage that was able to escape CRISPR-based immunity from Streptococcus thermophillus.

Based on self-targeting bioinformatics analysis, a bioinformatics pipeline named self-targeting spacer search (STSS) has recently been developed to predict the self-targeting sequence in all available bacterial genomes with the predicted CRISPR arrays. Using STSS combined with a functional screening system called transcription-cell-free translation (TXTL), the inhibitors of Cas12a, namely AcrVA1, AcrVA4, and AcrVA5 have been recently identified.

Within all the anti-CRISPR proteins that have been discovered so far, mechanisms have been described for only 15 of among them. These mechanisms can be divided into a number of different types: (1) crRNA loading interference, (2) DNA binding blockage, and (3) DNA cleavage prevention. Among these, crRNA (CRISPR RNA) loading interference mechanism has been mainly associated with the AcrIIC2 protein family. In order to block Cas9 activity, it prevents the correct assembly of the crRNA-Cas9 complex. In addition, AcrIJA5 appears to also inhibit CRISPR systems via gRNA disruption. More information in this regard can be found in, for example, a review by Zhang et al., Animal Model Exp Med. 2019 June; 2(2): 69-75, which is herein incorporated by reference.

With regard to DNA binding blockage, AcrIIC2 has been shown not to be the only one capable of blocking DNA binding. There are 11 other Acr family proteins that can also carry it out. Some among those are AcrIF1, AcrIF2, and AcrIF10, which act on different subunits of the Cascade effector complex of the type I-F CRISPR-Cas system, preventing the DNA to bind to the complex. Furthermore, AcrIIC3 prevents DNA binding by promoting dimerization of Cas9 and AcrIIA2 mimics DNA, thereby blocking the PAM recognition residues and consequently preventing dsDNA (double-stranded DNA) recognition and binding.

Regarding DNA cleavage prevention, AcrE1, AcrIF3 and AcrIIC1 can prevent target DNA cleavage. Using X-ray crystallography, AcrE1 was discovered to bind to the CRISPR associated Cas3. Likewise, biochemical and structural analysis of AcrIF3 showed its capacity of binding to Cas3 as a dimer so as to prevent the recruitment of Cas3 to the Cascade complex. From biochemical and structural AcrIIC1 studies, it was found that it binds to the active site of the HNH endonuclease domain in Cas9, which prevents DNA from cleaving. Thus, it turns Cas9 into an inactive but DNA bound state.

Based on these reports and biophysical and biochemical analyses, a picture has emerged by which these Acrs can inhibit CRISPR activity by a variety of mechanisms and with varying promiscuity, but predominantly specifically inhibit the binding of a small set of Cas proteins to DNA. These studies demonstrated inhibition of gene expression in E. coli cells or extracts, as well as inhibition of genomic editing imaging, or deposition of epigenetic marks. However, the broad extent as to whether Acrs can be used as tools to provide temporal, inhibitory control of CRISPR genome editors and nuclease-deactivated Cas9 (dCas9) genome regulators (both activation and repression) in different eukaryotic cells remains to be characterized. Described herein is a comprehensive characterization of Acr activity in a range of contexts and establish the basis for biotechnological applications involving the use of Acrs for controlling CRISPR activity in eukaryotic cells, e.g., mammalian cells.

### Compositions of the Disclosure

### Engineered Cells

In one aspect, provided herein are various engineered cells including: (a) one or more components of a CRISPR system including (i) a Cas endonuclease or a first nucleic acid encoding the Cas endonuclease, and (ii) at least one gRNA capable of binding to a target polynucleotide sequence in the cell; and (b) an anti-CRISPR (Acr) polypeptide or a second nucleic acid encoding the Acr polypeptide, wherein the Acr polypeptide modulates an activity of the CRISPR system in an inducible and/or programmable manner.

As discussed above, the skilled artisan in the art would understand that the term “programmable” when used in reference to a CRISPR system refers to the ability to engineer the nuclease-based platforms for recognizing and/or binding a specific target sequence in chromosomal DNA and modifying the DNA or protein(s) associated with DNA at or near the target sequence.

In principle, there are no particular limitations to the number of gRNA that can be included in the engineered cells of the disclosure. In some embodiments, the engineered cell includes one gRNA molecule that targets an activity of a to a CRISPR system to a specific target sequence in the genome. In some embodiments, the engineered cell includes a plurality of gRNAs that can effect multiplex editing or regulation of multiple target loci. Accordingly, in some embodiments, the engineered cell can include from one to twenty gRNAs, such as one, two, three, four, five, six, seven, eight, nine, or ten gRNAs.

In some embodiments, at least one gRNA is encoded by a sequence incorporated in the first nucleic acid, which also includes a coding sequence for the Cas endonuclease. In some embodiments, at least one gRNA is encoded by a sequence incorporated in the second nucleic acid, which also includes a coding sequence for the Acr polypeptide. In some embodiments, the CRISPR system further including a donor template that includes a sequence encoding a gene-of-interest. In some embodiments, the sequence encoding the gRNA and the sequence encoding the GOI are incorporated in the same nucleic acid molecule. In some embodiments, the sequence encoding the gRNA and the sequence encoding the GOI are incorporated in different nucleic acid molecules.

In some embodiments, the first nucleic acid and/or the first nucleic acid are incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, the CRISPR system includes a Cas endonuclease or a derivative thereof. As described above, a Cas derivative can be devoid of at least one of its activities, such as for example a Cas9 protein devoid of its nuclease activity, which is referred to as “dCas9.” A catalytically-inactive programmable RNA-dependent DNA-binding protein (e.g., dCas9) can be generated by mutating residues in the RuvC and HNH catalytic domains within Cas9. As such, CRISPR-associated catalytically inactive dCas protein can be used as a general platform for RNA-guided DNA targeting. For example, fusion of dCas to effector domains with distinct regulatory functions enables stable and efficient transcriptional repression or activation of target genes in an engineered cell, with the site of delivery determined solely by a co-expressed sgRNA.

In principle, there are no particular limitations to Cas endonucleases that can be used. In some embodiments, the CRISPR system includes a Class 2 Cas endonuclease or a derivative thereof. In some embodiments, the Class 2 Cas endonuclease is selected from the group consisting of Type II Cas endonucleases, Type IIA Cas endonucleases, Type BIB Cas endonucleases, Type IIC Cas endonucleases, Type V Cas endonucleases, Type VI Cas endonucleases, or a derivative of any thereof. In some embodiments, the Class 2 Cas endonuclease is a Type II Cas endonuclease or a derivative of any thereof. In some embodiments, the Class 2 Cas endonuclease is a Type IIA Cas endonuclease, a Type BB Cas endonuclease, a Type IIC Cas endonuclease, or a derivative of any thereof. In some embodiments, the Type II Cas endonuclease is a Cas9 endonuclease or a derivative thereof. In some embodiments, the Class 2 Cas endonuclease is a Type IIA Cas endonuclease or a derivative of any thereof. In some embodiments, the Type IIA Cas endonuclease is a Csn2 endonuclease or a derivative thereof.

In some embodiments, suitable Cas9 endonucleases include, but are not limited to, Streptococcus pyogenes Cas9, a Streptococcus thermophiles Cas9, a Staphylococcus aureus Cas9, a Brackiella oedipodis Cas9, a Neisseria meningitidis Cas9, a Haemophilus influenzae Cas9, a Simonsiella muelleri Cas9, a Ralstonia solanacearum Cas9. Additional non-limiting examples of Cas9 endonucleases suitable for the compositions and methods disclosed herein include Francisella novicida Cas9, and a Listeria monocytogenes Cas9. In some embodiments, the Cas9 derivative is a catalytically-inactive nuclease-deficient Cas9 polypeptide (dCas9).

In some embodiments, the Class 2 Cas endonuclease is a Type V Cas endonuclease or a derivative of any thereof. In some embodiments, the Type V Cas endonuclease is a Cas12 endonuclease or a derivative thereof. In some embodiments, the Type V Cas endonuclease is a Cas12 endonuclease selected from the group consisting of Cas12a, Cas12b, and Cas12c, or a derivative of any thereof. Suitable Cas12 endonucleases include, but are not limited to, Francisella novicida Cas12, a Lachnospiraceae bacterium ND2006 Cas12, and an Acidaminococcus sp. BV3L6 Cas12a. In some embodiments, the Cas12 derivative is a catalytically-inactive Cas12 polypeptide (e.g., nuclease-deficient Cas12 polypeptide; “dCas12”).

In some embodiments, the Cas endonuclease is operably linked to an effector domain. As used herein, the term “effector domain” refers to a protein domain that can: 1) affect either transcriptional repression or activation, 2) catalytically modify histones, or 3) catalytically chemically modify DNA. Accordingly, in some embodiments, the Cas endonuclease of the CRISPR system is operably linked to one or more effector domains. Non-limiting examples of effector domains suitable for the compositions and methods disclosed herein include activation domains, repression domains, protein modification domains, histone modification domains, DNA modification domains, and RNA modification domains. In some embodiments, heterodimerization domains for controlling localization of genomic loci to subcellular regions can suitably be used as effector domains in the compositions and methods described herein. As discussed above, fusion of dCas9 to effector domains with distinct regulatory functions enables stable and efficient transcriptional repression or activation in a target cell, with the site of delivery determined solely by a co-expressed sgRNA.

In some embodiments, the CRISPR system is a CRISPR-based gene regulation system for activation (CRISPRa) or repression (CRISPRi) of target genes in an engineered cell, with the site of delivery determined solely by a co-expressed sgRNA.

In some embodiments, a CRISPR-based gene activation (CRISPRa) system can be used to mediate activation of one or more target genes in the cell. In some embodiments, the CRISPRa system includes a dCas9 polypeptide operably linked to an activation domain. In some embodiments, the CRISPRa system includes a dCas12 polypeptide operably linked to an activation domain. The activation domain can be a transcriptional activation domain. Non-limiting examples of suitable transcriptional activation domains include VP16 activation domain, VP64 activation domain, p65 activation domain, MyoD1 activation domain, HSF1 activation domain, RTA activation domain, SETT/9 activation domain, VP64-p65-Rta (VPR) activation domain, a mini VPR activation domain, yeast GAL4 activation domain, yeast HAP1 activation domain, and a histone acetyltransferase.

In some embodiments, a CRISPR-based gene interference (CRISPRi) system can be used to enable downregulation of one or more target genes in the cell. In some embodiments, the CRISPRi system includes a dCas9 polypeptide operably linked to a repression domain. In some embodiments, the CRISPRi system includes a dCas12 polypeptide operably linked to a repression domain. The repression domain can be a transcriptional repression domain. In some embodiments, the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3A repressor domain, and a DNMT3L repressor domain.

In some embodiments, the CRISPR system can be used to edit a genome is a CRISPR-based genome editing system. One or more activities of the CRISPR-based genome editing system can be modulated by the Acr polypeptide in the engineered cell. Such activities include, but are not limited to, target polynucleotide binding, target polynucleotide double-strand break creation, target polynucleotide nicking (single-strand DNA cleavage), and target polynucleotide modification. In some embodiments, one or more CRISPR-mediated modifications of the target locus is modulated. Non-limiting examples of CRISPR-mediated modifications of the target polynucleotide include insertion of at least one nucleotide, deletion of at least one nucleotide, substitution of at least one nucleotide, and chemical alteration of at least one nucleotide at, within, or near the target locus.

As discussed above, small proteins encoded by bacteriophages have been shown to inhibit the CRISPR-Cas systems of host bacteria. These naturally occurring inhibitors can potentially inactivate CRISPR-Cas systems when needed, or decrease off-target effects, without significantly affecting on-target efficiency. Thus, these proteins can be used as a promising method to control CRISPR-Cas activity. In some embodiments, the Acr polypeptides are bacteriophage-derived Acr polypeptides. Non-limiting examples of bacteriophage-derived Acr polypeptides include AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof. In some embodiments, the Acr polypeptide is selected from the group consisting of AcrIIA4, AcrIIA5, and AcrIIA6 or a functional variant thereof. In some embodiments, the Acr polypeptide is AcrIIA4.

In some embodiments, the Acr functional variants have at least 80% sequence identity, for examples at least 85%, at least 90%, at least 95%, at least 96%, at least 97%, at least 98%, or at least 99% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, and AcrVA5. In some embodiments, the Acr functional variants have at least 80% sequence identity, for examples at least 85%, at least 90%, at least 95%, at least 96%, at least 97%, at least 98%, or at least 99% sequence identity to AcrIJA4. In some embodiments, the Acr functional variants have at least 80% sequence identity, for examples at least 85%, at least 90%, at least 95%, at least 96%, at least 97%, at least 98%, or at least 99% sequence identity to AcrIIA5. In some embodiments, the Acr functional variants have at least 80% sequence identity, for examples at least 85%, at least 90%, at least 95%, at least 96%, at least 97%, at least 98%, or at least 99% sequence identity to AcrIIA6.

In some of such embodiments, the polynucleotide sequences encoding the Cas endonuclease and the Acr polypeptide are codon-optimized according to methods standard in the art for expression in the cell containing the target DNA of interest. For example, if the intended target nucleic acid is in a human cell, a human codon-optimized polynucleotide sequence encoding Cas9 and/or a human codon-optimized polynucleotide sequence encoding Acr polypeptide are contemplated for use.

In some embodiments, the expression and/or activity of the Acr polypeptide and/or the CRISPR system is inducible or programmable. For example, in some embodiments, the stability and/or activity of the Acr polypeptide and/or the Cas endonuclease can be modulated by an inducible protein degradation approach. In some embodiments, the expression and/or activity of the Acr polypeptide and/or the Cas endonuclease can be modulated via incorporation of one or more inducible DD in the respective polypeptides, wherein binding of a stabilizing ligand to the destabilization domain(s) incorporated in the Acr polypeptide and/or the Cas endonuclease prevents proteasomal degradation of the DD-polypeptide fusion, which in turn allows dosable control of the CRISPR system (as illustrated in, e.g., Example 7). The inducible DD(s) can be independently incorporated in the Acr polypeptide and/or the Cas endonuclease at its C-terminus, N-terminus, and/or internally. The inducible DD(s) can be independently incorporated in the Acr polypeptide and/or the Cas endonuclease within the C-terminal regions, e.g., fused to the C-termini of the respective polypeptides. The inducible DD(s) can be independently incorporated in the Acr polypeptide and/or the Cas endonuclease within the N-terminal regions, e.g., fused to the N-termini of the respective polypeptides. The inducible DD(s) can be independently incorporated in the Acr polypeptide and/or the Cas endonuclease within the sequences of the respective polypeptides. In some embodiments, the Acr polypeptide is operably linked to at least one inducible DD. In some embodiments, the Cas endonuclease polypeptide is operably linked to an inducible DD. In some embodiments, at least one inducible DD is incorporated in each of the Acr polypeptide and the Cas endonuclease polypeptide. The inducible DD incorporated in the Acr polypeptide and the Cas endonuclease polypeptide can be the same or can be different. Suitable destabilization domains include, but are not limited to, a DD from the FKBP12, a DD from dihydrofolate reductase (DHFR), a DD from estrogen receptor ligand binding domain (ERLBD), and a DD from estrogen receptor (ER50). In some embodiments, engineered versions of the FKB12 domain can also be chemically induced to be directly targeted to endogenous ubiquitination system as alternative inducible protein degradation approach. Additional non-limiting examples of inducible DDs suitable for the compositions and methods disclosed herein include the AID-auxin system and NS3pro-N4a, as well as peptide-based caging of constitutive degrons such as the cODC degron.

In some embodiments, the inducible DD is derived from FKBP12 and the stabilization of the DD is induced by addition of stabilizing ligand rapamycin analogue compound Shield1, which in turn creates an integrated Shield1-inducible CRISPRa or CRISPRi system. In some embodiments, the inducible DD is derived from DHFR and the stabilization of the DD is induced by addition of stabilizing ligand trimethoprim (TMP), which in turn creates an integrated TMP-inducible CRISPRa or CRISPRi system. In some embodiments, the inducible DD is derived from estrogen receptor (ER50) and the stabilization of the DD is induced by addition of stabilizing ligand 4-hydroxytamoxifen (4HT or 40HT) or CMP8, which in turn creates an integrated 4HT or 40HT-inducible CRISPRa or CRISPRi system.

In some embodiments, at least one destabilization domain is fused to a Cas-endonuclease, wherein binding of the stabilizing ligand to the destabilization domain increases activity of the Cas-endonuclease (e.g., ligand-inducible activation) by at least 30%, at least 40%, at least 50%, at least 60%, at least 70%, at least 80%, at least 90%, or at least 100%. In some embodiments, the binding of the stabilizing ligand to the destabilization domain increases activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, at least one destabilization domain is fused to a Acr polypeptide that counteract the activity of a Cas-endonuclease, wherein binding of the stabilizing ligand to the destabilization domain decreases the activity of the Cas-endonuclease (e.g., ligand-inducible inhibition) by at least 30%, at least 40%, at least 50%, at least 60%, at least 70%, at least 80%, at least 90%, or at least 100%. In some embodiments, the binding of the stabilizing ligand to the destabilization domain decreases the activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, the expression and/or activity of the Acr polypeptide and/or the Cas endonuclease can be modulated via incorporation of one or more proteolytic cleavage sites. In some embodiments, the Acr polypeptide is operably linked to at least one proteolytic cleavage site. In some embodiments, the Cas endonuclease is operably linked to at least one proteolytic cleavage site. In principle, there are no particular limitations to the proteolytic cleavage sites that can be used. Suitable examples of proteolytic cleavage sites include, but are not limited to, sequences that can be cleaved by a tobacco etch virus (TEV) protease, a porcine teschovirus-1 2A (P2A) protease, a foot-and-mouth disease virus (FMDV) 2A (F2A) protease, an Equine Rhinitis A Virus (ERAV) 2A (E2A) protease, a Thosea asigna virus 2A (T2A) protease, a cytoplasmic polyhedrosis virus 2a (BmCPV2A) protease, a Flacherie Virus 2A (BmIFV2A) protease. Additional non-limiting proteolytic cleavage sites suitable for this purpose include thrombin, PreScission™ protease, a glutamyl endopeptidase, an Epstein-Barr virus protease, a matrix metalloproteinase 2 (MMP-2), a matrix metalloproteinase 1 (MMP-1), a membrane type 1 matrixmetalloproteinase (MT-MMP), a stromelysin 3 (or MMP-11), a matrix metalloproteinase 13 (collagenase-3), an MMP-3 (stromelysin), an MMP-7 (matrilysin), MMP-9, an NS3 protease, and a thermolysin-like MMP.

In some embodiments, at least one proteolytic cleavage site is incorporated into a Cas-endonuclease, wherein cleavage of the at least one proteolytic cleavage site increases activity of the Cas-endonuclease (e.g., ligand-inducible activation) by at least 30%, at least 40%, at least 50%, at least 60%, at least 70%, at least 80%, at least 90%, or at least 100%. In some embodiments, the binding of the stabilizing ligand to the destabilization domain increases activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, at least one proteolytic cleavage site is incorporated into a Acr polypeptide that counteract the activity of a Cas-endonuclease, wherein cleavage of the at least one proteolytic cleavage site decreases the activity of the Cas-endonuclease (e.g., ligand-inducible inhibition) by at least 30%, at least 40%, at least 50%, at least 60%, at least 70%, at least 80%, at least 90%, or at least 100%. In some embodiments, the binding of the stabilizing ligand to the destabilization domain decreases the activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, the Acr polypeptide and/or Cas-endonuclease can be further linked to one or more additional domains such as, e.g. a nuclear localization signal (NLS), a G-protein-coupled receptor (GPCR), a flexible Gly-Ser linker, and a synthetic Notch receptor. As described below in Example 6 below, GPCR activation by cognate ligand binding induces recruitment of β-arrestin, which allows for release of dCas9-VPR for nuclear localization and gene activation. According, the activity and/or expression of the Acr polypeptide can be further modulated via incorporation of a GPCR polypeptide to create an integrated doxycycline-inducible CRISPRa or CRISPRi system.

In some embodiments, the Acr polypeptide and/or Cas-endonuclease can be operably linked to a synthetic Notch receptor. Notch receptors are transmembrane proteins that mediate cell-cell contact signaling and play a central role in development and other aspects of cell-to-cell communication. Many synthetic Notch receptors are known in the art and generally replace the extracellular ligand-binding domain, which in wild-type Notch contains multiple EGF-like repeats, with an antibody derivative, and replacing the cytoplasmic domain with a transcription activator of choice, while still relying on the Notch negative regulatory region (NRR). Additional information in this regard can be found in, for example, Roybal L. et al., Cell (2016) 164:780-91; and Roybal K. T. et al., Cell. 2016 Oct. 6; 167(2): 419-432.

Attachment or association of the various domains described in the compositions and methods of the disclosure can be achieved via a linker, e.g., a flexible glycine-serine (GlyGlyGlySer) or (GGGS)₃ or a rigid alpha-helical linker such as (Ala(GluAlaAlaAlaLys)Ala). Linkers such as (GGGGS)₃ are preferably used herein to separate protein or peptide domains. (GGGGS)₃ is preferable because it is a relatively long linker (15 amino acids). The glycine residues are the most flexible and the serine residues enhance the chance that the linker is on the outside of the protein. (GGGGS)₆ (GGGGS)₉ or (GGGGS)₁₂ may preferably be used as alternatives. Other preferred alternatives are (GGGGS)₁, (GGGGS)₂, (GGGGS)₄, (GGGGS)₅, (GGGGS)₇, (GGGGS)₈, (GGGGS)₁₀, or (GGGGS)n. Alternative linkers are available, but highly flexible linkers are thought to work best to allow for maximum opportunity for the 2 parts of the Acr polypeptide and/or Cas-endonuclease polypeptide to come together and thus reconstitute a desired activity. One alternative is that the NLS of nucleoplasmin can be used as a linker. For example, a linker can also be used between the Acr polypeptide and/or Cas-endonuclease and any functional domain. Again, a (GGGGS)₃ linker may be used here (or the 6, 9, or 12 repeat versions therefore) or the NLS of nucleoplasmin can be used as a linker between the Acr polypeptide and/or Cas-endonuclease and the functional domain.

As discussed above, the nucleic acids encoding the Acr polypeptide and/or the CRISPR system can be stably integrated in the host genome, or can be episomally replicating, or present in the engineered cell as a mini-circle expression vector for transient expression. Accordingly, in some embodiments disclosed herein, the nucleic acids are maintained and replicated in the engineered cell as an episomal unit, and the Acr polypeptide and/or one or more components of the CRISPR system are expressed episomally in the cell. In some other embodiments, the nucleic acids are stably integrated into the genome of the recombinant cell, wherein the Acr polypeptide and/or the one or more components of the CRISPR system are expressed from nucleic acids stably integrated in the genome of the cell.

In some embodiments, the expression and/or activity of the Acr polypeptide and/or the Cas endonuclease can be modulated at transcriptional level. Accordingly, in some embodiments, the expression of the Acr polypeptide and/or the one or more components of the CRISPR system is under control of a promoter. Non-limiting examples of suitable eukaryotic promoters (e.g., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), mouse metallothionein-I, and chicken β-Actin promoter coupled with CMV early enhancer (CAGG).

In some embodiments, the expression of the Acr polypeptide and/or the one or more components of the CRISPR system is under control of a promoter constitutive promoter. In principle, there are no particular limitations to the constitutive promoters that can be used. Non-limiting examples of suitable constitutive promoters include SV40 promoters, CMV promoters, PGK promoters, ubiquitin C (UBC) promoters, EF1A promoters, CAGG promoters, SFFV promoters. In some embodiments, the constitutive promoter is a CMV constitutive promoter. In some embodiments, the constitutive promoter is a PGK constitutive promoter.

In some embodiments, the expression of the Acr polypeptide and/or the one or more components of the CRISPR system is under control of a repressible promoter or an inducible promoter. In principle, the inducible promoter can generally be any the inducible promoter and can be, for example, a TRE3G inducible promoter, a tetracycline-regulated promoter, a steroid-regulated promoter, a metal-regulated promoter, an estrogen receptor-regulated promoter, or an inducible UAS promoter. In some embodiments, the inducible promoter is a TRE3G inducible promoter. In some embodiments, the inducible promoter is a tetracycline inducible promoter.

In some embodiments, the expression of the Acr polypeptide and/or the one or more components of the CRISPR system is under control of the same promoter. In some embodiments, the promoter is a spatially restricted and/or temporally restricted promoter such as, a tissue specific promoter, a cell type specific promoter, a growth-stage specific promoter, etc.

In some embodiments, the engineered cell of the disclosure includes: (a) a first nucleic acid molecule encoding (i) an Acr polypeptide and (ii) a sgRNA; and (b) a second nucleic acid molecule encoding a catalytically inactive Cas polypeptide (e.g., nuclease-deficient Cas polypeptide; “dCas”) fused to an effector domain, wherein the Acr polypeptide inhibits the activity of the dCas-effector domain fusion and wherein the expression of the Acr polypeptide and the CRISPRa system is under control of the same promoter. In some embodiments, the first nucleic acid molecule encoding the Acr polypeptide and the sgRNA is stably integrated into the genome of the cell. In some embodiments, the first nucleic acid molecule the first nucleic acid molecule encoding the Acr polypeptide and the sgRNA is maintained episomally in the cell.

The methods and compositions disclosed herein may be deployed for engineering cells of any species, including, but not limited to, prokaryotic and eukaryotic species. Suitable host cells to be engineered according to the present disclosure can include, but not limited to, algal cells, bacterial cells, fungal cells, plant cells, and animal cells. In some embodiments, the cell is an animal cell. In some embodiments, the animal cell is an invertebrate animal cell. In some embodiments, the vertebrate animal cell is a mammalians cell. In some embodiments, the mammalian cell is a human cell. In some embodiments, the human cell is a stem cell. In some embodiments, the stem cell is a human-induced pluripotent stem cell (hiPSC).

Introduction of the polypeptides and nucleic acid molecules of the disclosure into cells can be achieved by any well-established method known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like. Host cells can be either untransformed cells or cells that have already been transfected with at least one nucleic acid molecule.

### Methods of Modulating Activity of Crispr Systems

As outlined above, experimental results presented herein demonstrate that Acr polypeptides can be used in development of new tools for programmable inhibition of CRISPR genome editors and catalytically inactive Cas9 polypeptide (e.g., nuclease-deficient Cas9 polypeptide; “dCas9”) genome regulators (e.g., activation and repression) in various target cells. This is because the advent of CRISPR technologies has drastically reduced the barrier of genome editing and control over gene expression. While undoubtedly a boon to science, this ease of use has also raised concomitant fears of the use of genome editing for malignant or otherwise unethical or illegal purposes. The results of certain experiments described herein demonstrated that cells pre-engineered with Arc molecules become resistant to genome editing, thus providing a means to generate “write-protected” cells that prevent future genome editing. These data provide a proof-of-principle demonstration of the practicality of integrating Acrs in the genome to generate cells that are immune to unlicensed editing applications.

In one aspect, some embodiments of the disclosure relate to various methods for selectively modulating an activity of a CRISPR system in a cell. The methods include providing to the cell: (a) one or more components of a CRISPR system including (i) a Cas endonuclease or a first nucleic acid encoding the Cas endonuclease, and (ii) at least one gRNA capable of binding to a target polynucleotide sequence in the cell; and (b) an anti-CRISPR (Acr) polypeptide or a second nucleic acid encoding the Acr polypeptide, wherein the Acr polypeptide modulates an activity of the CRISPR system in an inducible and/or programmable manner.

“Providing” (and “provided”) is intended to mean presenting to the host cell, any polypeptides, ribonucleoprotein (RNP) complexes, and/or nucleic acid molecules used in the methods provided herein, e.g. nucleic acids encoding Cas endonucleases, gRNAs, and/or Acr polypeptides as described herein, in such a manner that the component(s) gains access to the interior of the host cell. The methods and compositions described herein do not depend on a particular method for introducing a nucleic acid or polypeptide into a host cell, only that the nucleic acid or polypeptide gains access to the interior of the host cell. Providing includes the introduction or incorporation of a nucleic acid or polypeptide into the host cell where the nucleic acid or polypeptide may be integrated into the genome of the host cell, and includes the transient provision (e.g., delivery) of a nucleic acid or polypeptide to the host cell.

Providing the polypeptides, nucleic acids, and RNP complexes of the disclosure into cells can be carried out by several methods known in the art, such as by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and similar techniques.

In some embodiments, any polypeptides, ribonucleoprotein (RNP) complexes, and/or nucleic acid molecules used in the methods provided herein, e.g. nucleic acids encoding Cas endonucleases, gRNAs, and/or Acr polypeptides described herein can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

In some embodiments of the disclosed methods, gRNAs and nucleic acids encoding Cas endonucleases, gRNAs, and/or Acr polypeptides can be delivered by viral or non-viral delivery vehicles known in the art. Alternatively, Cas endonuclease(s) and Acr polypeptide(s) can be delivered by viral or non-viral delivery vehicles known in the art, such as electroporation or lipid nanoparticles. In some embodiments, the Cas endonuclease(s) can be delivered as one or more polypeptides, either alone or pre-complexed with one or more guide RNAs, or one or more crRNA together with a tracrRNA.

In some embodiments, nucleic acids can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

In some embodiments, the introduction of a nucleic acid can be stable, e.g., that the nucleic acid (construct or vector) introduced into host cell integrates into a genome of the host cell and is capable of being inherited by the progeny thereof. In some other embodiments, the introduction can be temporary, e.g., that the nucleic acid (construct or vector) is introduced into the host cell and does not integrate into a genome of the host cell. Transient transformation indicates that the introduced nucleic acid or protein is only temporarily expressed or present in the host cell.

In some embodiments, a polypeptide such as a Cas endonuclease or Acr polypeptide can be introduced into a host cell by directly introducing the polypeptide itself or an mRNA encoding the polypeptide. In some embodiments, the polypeptide can be introduced into a host cell transiently. In some embodiments, uptake of the polypeptide into the host cell can be facilitated with a cell penetrating peptide (CPP).

In principle, there are no particular limitations to the number of gRNA that can be included in the engineered cells of the disclosure. In some embodiments, the engineered cell includes one gRNA molecule that targets an activity of a CRISPR system to a specific target polynucleotide sequence in the genome. In some embodiments, the engineered cell includes a plurality of gRNAs that can effect multiplex editing or multiplex regulation of a plurality of target loci. Accordingly, in some embodiments, it is contemplated that the engineered cell can include from one to twenty gRNAs, such as one, two, three, four, five, six, seven, eight, nine, or ten gRNAs.

In some embodiments, at least one gRNA is encoded by a sequence incorporated in the first nucleic acid, which also includes a coding sequence for the Cas endonuclease. In some embodiments, at least one gRNA is encoded by a sequence incorporated in the second nucleic acid, which also includes a coding sequence for the Acr polypeptide. In some embodiments, the CRISPR system further including a donor template that includes a sequence encoding a gene-of-interest. In some embodiments, the sequence encoding the gRNA and the sequence encoding the GOI are incorporated in the same nucleic acid molecule. In some embodiments, the sequence encoding the gRNA and the sequence encoding the GOI are incorporated in different nucleic acid molecules.

In some embodiments, the first nucleic acid and/or the second nucleic acid are incorporated into an expression vector, which can be stably integrated in the host genome, or can be episomally replicating, or can be present in the cell as a mini-circle expression vector for transient expression. Accordingly, in some embodiments, the vector can be a replicon, such as a plasmid, phage, or cosmid, into which another DNA segment may be inserted so as to bring about the replication of the inserted segment. In some embodiments, the expression vector can be an integrating vector. In some embodiments, the expression vector can be a viral vector which, as discussed above, may refer either to a virus or viral particle capable of transferring a nucleic acid into a cell or to the transferred nucleic acid itself. In some embodiments, the expression vector is a retroviral vector. In some embodiments, the viral vector is a lentiviral vector with structural and functional genetic elements, or portions thereof, including LTRs that are primarily derived from a lentivirus, which is a genus of retrovirus.

In some embodiments of the disclosed methods, the CRISPR system includes a Cas9 endonuclease or a derivative thereof. As described above, a Cas derivative can be devoid of at least one of its activities, such as for example a Cas9 protein devoid of its nuclease activity, which is referred to as “dCas9.” A catalytically-inactive programmable RNA-dependent DNA-binding protein (e.g., dCas9) can be generated by mutating residues in the RuvC and HNH catalytic domains within Cas9. As such, CRISPR-associated catalytically inactive dCas9 protein can be used as a general platform for RNA-guided DNA targeting. For example, fusion of dCas9 to effector domains with distinct regulatory functions enables stable and efficient transcriptional repression or activation of target genes in an engineered cell, with the site of delivery determined solely by a co-expressed sgRNA.

In principle, there are no particular limitations to Cas endonucleases that can be use in the methods described herein. In some embodiments, the CRISPR system includes a Class 2 Cas endonuclease or a derivative thereof. In some embodiments, the Class 2 Cas endonuclease is selected from the group consisting of Type II Cas endonucleases, Type IIA Cas endonucleases, Type BB Cas endonucleases, Type IIC Cas endonucleases, Type V Cas endonucleases, Type VI Cas endonucleases, or a derivative of any thereof. In some embodiments, the Class 2 Cas endonuclease is a Type II Cas endonuclease or a derivative of any thereof. In some embodiments, the Class 2 Cas endonuclease is a Type IIA Cas endonuclease, a Type IIB Cas endonuclease, a Type IIC Cas endonuclease, or a derivative of any thereof. In some embodiments, the Type II Cas endonuclease is a Cas9 endonuclease or a derivative thereof. In some embodiments, the Class 2 Cas endonuclease is a Type IIA Cas endonuclease or a derivative of any thereof. In some embodiments, the Type IIA Cas endonuclease is a Csn2 endonuclease or a derivative thereof. Non-limiting examples of suitable Cas9 endonucleases include Streptococcus pyogenes Cas9, a Streptococcus thermophiles Cas9, a Staphylococcus aureus Cas9, a Brackiella oedipodis Cas9, a Neisseria meningitidis Cas9, a Haemophilus influenzae Cas9, a Simonsiella muelleri Cas9, and a Ralstonia solanacearum Cas9. Additional examples of Cas9 endonucleases suitable for the methods disclosed herein include, but are not limited to Francisella novicida Cas9, and a Listeria monocytogenes Cas9. In some embodiments, the Cas9 derivative is a nuclease-deficient Cas9 polypeptide (dCas9).

In some embodiments, the Class 2 Cas endonuclease is a Type V Cas endonuclease or a derivative of any thereof. In some embodiments, the Type V Cas endonuclease is a Cas12 endonuclease or a derivative thereof. In some embodiments, the Type V Cas endonuclease is a Cas12 endonuclease selected from the group consisting of Cas12a, Cas12b, and Cas12c, or a derivative of any thereof. Suitable Cas12 endonucleases include, but are not limited to, Francisella novicida Cas12, a Lachnospiraceae bacterium ND2006 Cas12, and an Acidaminococcus sp. BV3L6 Cas12a. In some embodiments, the Cas9 derivative is a nuclease-deficient Cas12 polypeptide (dCas12).

In some embodiments, the Cas endonuclease is operably linked to an effector domain. As used herein, the term “effector domain” refers to a protein domain that can: 1) affect either transcriptional repression or activation, 2) catalytically modify histones, or 3) catalytically chemically modify DNA. Accordingly, in some embodiments, the Cas endonuclease of the CRISPR system is operably linked to one or more effector domains. Non-limiting examples of effector domains suitable for the compositions and methods disclosed herein include activation domains, repression domains, protein modification domains, histone modification domains, DNA modification domains, and RNA modification domains. In some embodiments, heterodimerization domains for controlling localization of genomic loci to subcellular regions can suitably be used as effector domains in the compositions and methods described herein. As discussed above, fusion of dCas to effector domains with distinct regulatory functions enables stable and efficient transcriptional repression or activation in a target cell, with the site of delivery determined solely by a co-expressed sgRNA.

Some embodiments of the disclosure relates to a method for modulating an activity of a CRISPR system which is a CRISPR-based gene regulation system. The skilled artisan in the art will understand that the term “modulating”, when used to in reference to an activity of a CRISPR system can refers to increasing (e.g., activation) or decreasing (inhibition) or otherwise affecting an activity of the CRISPR system. Accordingly, some embodiments of the disclosure relates to a method for modulating an activity of a CRISPR-based gene regulation system which is a CRISPR-based gene activation (CRISPRa) system, wherein a Cas endonuclease of the CRISPRa is operably linked to an activation domain. In some embodiments, a CRISPRa system can be used to mediate activation (e.g., upregulation) of one or more target genes in the cell. In some embodiments, the CRISPRa system includes a dCas9 polypeptide operably linked to an activation domain. In some embodiments, the CRISPRa system includes a dCas12 polypeptide operably linked to an activation domain. The activation domain can be a transcriptional activation domain. Non-limiting examples of suitable transcriptional activation domains include VP16 activation domain, VP64 activation domain, p65 activation domain, MyoD1 activation domain, HSF1 activation domain, RTA activation domain, SETT/9 activation domain, VP64-p65-Rta (VPR) activation domain, a mini VPR activation domain, yeast GAL4 activation domain, yeast HAP1 activation domain, and a hi stone acetyltransferase.

Some embodiments of the disclosure relates to a method for modulating an activity of a CRISPR-based gene regulation system which is a CRISPR-based gene interference (CRISPRi) system, wherein a Cas endonuclease of the CRISPRi is operably linked to a repressor domain. In some embodiments, a CRISPRi system can be used to enable downregulation of one or more target genes in the cell. In some embodiments, the CRISPRi system includes a dCas9 polypeptide operably linked to a repression domain. In some embodiments, the CRISPRi system includes a dCas12 polypeptide operably linked to a repression domain. The repression domain can be a transcriptional repression domain. In some embodiments, the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3A repressor domain, and a DNMT3L repressor domain.

Some embodiments of the disclosure relates to a method for modulating an activity of a CRISPR system which can be used to edit a genome, also referred herein as a CRISPR-based genome editing system. One or more activities of the CRISPR-based genome editing system can be modulated by the Acr polypeptide in the engineered cell. Such activities include, but are not limited to, target polynucleotide binding, target polynucleotide double-strand break creation, target polynucleotide nicking (single-strand DNA cleavage), and target polynucleotide modification. In some embodiments, one or more CRISPR-mediated modifications of the target locus is modulated. Non-limiting examples of CRISPR-mediated modifications of the target polynucleotide include insertion of at least one nucleotide, deletion of at least one nucleotide, substitution of at least one nucleotide, and chemical alteration of at least one nucleotide at, within, or near the target locus.

As discussed above, small proteins encoded by bacteriophages have been shown to inhibit the CRISPR-Cas systems of host bacteria. These naturally occurring inhibitors can potentially inactivate CRISPR-Cas systems when needed, or decrease off-target effects, without significantly affecting on-target efficiency. Thus, these proteins can be used as a promising method to control CRISPR-Cas activity. In some embodiments, the Acr polypeptides are bacteriophage-derived Acr polypeptides. Non-limiting examples of bacteriophage-derived Acr polypeptides include AcrIIC1, AcrIIA1, AcrIIC2, AcrIIC3, AcrIIA2, AcrIIA3, AcrIIA4, AcrIIA5, AcrIIA6, AcrVA1, AcrVA2, AcrVA3, AcrVA4, AcrVA5, and functional variants of any thereof. In some embodiments, the Acr polypeptide is selected from the group consisting of AcrIIA4, AcrIIA5, and AcrIIA6 or a functional variant thereof. In some embodiments, the Acr polypeptide is AcrIIA4.

In some embodiments, the Acr functional variants have at least 80% sequence identity, for examples at least 85%, at least 90%, at least 95%, at least 96%, at least 97%, at least 98%, or at least 99% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6 AcrVA1, AcrVA2, AcrVA3, AcrVA4, and AcrVA5. In some embodiments, the Acr functional variants have at least 80% sequence identity, for examples at least 85%, at least 90%, at least 95%, at least 96%, at least 97%, at least 98%, or at least 99% sequence identity to AcrIIA4. In some embodiments, the Acr functional variants have at least 80% sequence identity, for examples at least 85%, at least 90%, at least 95%, at least 96%, at least 97%, at least 98%, or at least 99% sequence identity to AcrIIA5. In some embodiments, the Acr functional variants have at least 80% sequence identity, for examples at least 85%, at least 90%, at least 95%, at least 96%, at least 97%, at least 98%, or at least 99% sequence identity to AcrIIA6.

In some of such embodiments, the polynucleotide sequences encoding the Cas endonuclease and the Acr polypeptide are codon-optimized according to methods standard in the art for expression in the cell containing the target DNA of interest. For example, if the intended target nucleic acid is in a human cell, a human codon-optimized polynucleotide sequence encoding Cas9 and/or a human codon-optimized polynucleotide sequence encoding Acr polypeptide are contemplated for use.

In some embodiments, the expression and/or activity of the Acr polypeptide and/or the CRISPR system is inducible or programmable. For example, in some embodiments, the stability and/or activity of the Acr polypeptide and/or the Cas endonuclease can be modulated by an inducible protein degradation approach. In some embodiments, the expression and/or activity of the Acr polypeptide and/or the Cas endonuclease can be modulated via incorporation of one or more inducible destabilization domains (DD) in the respective polypeptides, wherein binding of a stabilizing ligand to the destabilization domain(s) incorporated in the Acr polypeptide and/or the Cas endonuclease prevents proteasomal degradation of the DD-polypeptide fusion, which in turn allows dosable control of the CRISPR system (as illustrated in, e.g., Example 7). The inducible DD(s) can be incorporated in the Acr polypeptide and/or the Cas endonuclease at its C-terminus, N-terminus, and/or internally. The inducible DD(s) can be independently incorporated in the Acr polypeptide and/or the Cas endonuclease within the C-terminal regions, e.g., fused to the C-termini of the respective polypeptides. The inducible DD(s) can be independently incorporated in the Acr polypeptide and/or the Cas endonuclease within the N-terminal regions, e.g., fused to the N-termini of the respective polypeptides. The inducible DD(s) can be independently incorporated in the Acr polypeptide and/or the Cas endonuclease within the sequences of the respective polypeptides. In some embodiments, the Acr polypeptide is operably linked to at least one inducible DD. In some embodiments, the Cas endonuclease polypeptide is operably linked to an inducible DD. In some embodiments, at least one inducible DD is incorporated in each of the Acr polypeptide and the Cas endonuclease polypeptide. The inducible DD incorporated in the Acr polypeptide and the Cas endonuclease polypeptide can be the same or can be different. In some embodiments, engineered versions of the FKB12 domain can also be chemically induced to be directly targeted to endogenous ubiquitination system as alternative inducible protein degradation approach. Additional non-limiting examples of inducible DDs include the AID-auxin system and NS3pro-N4a, as well as peptide-based caging of constitutive degrons such as the cODC degron.

In some embodiments, the inducible DD is derived from FKBP12 and the stabilization of the DD is induced by addition of rapamycin analogue compound Shield1, which in turn creates an integrated Shield1-inducible CRISPRa or CRISPRi system. In some embodiments, the inducible DD is derived from DHFR and the stabilization of the DD is induced by addition of stabilizing ligand trimethoprim (TMP), which in turn creates an integrated TMP-inducible CRISPRa or CRISPRi system. In some embodiments, the inducible DD is derived from estrogen receptor (ER50) and the stabilization of the DD is induced by addition of stabilizing ligand 4-hydroxytamoxifen (4HT or 40HT) or CMP8, which in turn creates an integrated 4HT or 40HT-inducible CRISPRa or CRISPRi system.

In some embodiments, at least one destabilization domain is fused to a Cas-endonuclease, wherein binding of the stabilizing ligand to the destabilization domain increases activity of the Cas-endonuclease (e.g., ligand-inducible activation) by at least 30%, at least 40%, at least 50%, at least 60%, at least 70%, at least 80%, at least 90%, or at least 100%. In some embodiments, the binding of the stabilizing ligand to the destabilization domain increases activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, at least one destabilization domain is fused to a Acr polypeptide that counteract the activity of a Cas-endonuclease, wherein binding of the stabilizing ligand to the destabilization domain decreases the activity of the Cas-endonuclease (e.g., ligand-inducible inhibition) by at least 30%, at least 40%, at least 50%, at least 60%, at least 70%, at least 80%, at least 90%, or at least 100%. In some embodiments, the binding of the stabilizing ligand to the destabilization domain decreases the activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, the expression and/or activity of the Acr polypeptide and/or the Cas endonuclease can be modulated via incorporation of one or more proteolytic cleavage sites. In some embodiments, the Acr polypeptide is operably linked to at least one proteolytic cleavage site. In some embodiments, the Cas endonuclease is operably linked to at least one proteolytic cleavage site. In principle, there are no particular limitations to the proteolytic cleavage sites that can be used. Suitable examples of proteolytic cleavage sites include, but are not limited to, sequences that can be cleaved by a tobacco etch virus (TEV) protease, a porcine teschovirus-1 2A (P2A) protease, a foot-and-mouth disease virus (FMDV) 2A (F2A) protease, an Equine Rhinitis A Virus (ERAV) 2A (E2A) protease, a Thosea asigna virus 2A (T2A) protease, a cytoplasmic polyhedrosis virus 2a (BmCPV2A) protease, a Flacherie Virus 2A (BmIFV2A) protease. Additional non-limiting proteolytic cleavage sites suitable for this purpose include thrombin, PreScission™ protease, a glutamyl endopeptidase, an Epstein-Barr virus protease, a matrix metalloproteinase 2 (MMP-2), a matrix metalloproteinase 1 (MMP-1), a membrane type 1 matrixmetalloproteinase (MT-MMP), a stromelysin 3 (or MMP-11), a matrix metalloproteinase 13 (collagenase-3), an MMP-3 (stromelysin), an MMP-7 (matrilysin), MMP-9, an NS3 protease, and a thermolysin-like MMP.

In some embodiments, at least one proteolytic cleavage site is incorporated into a Cas-endonuclease, wherein cleavage of the at least one proteolytic cleavage site increases activity of the Cas-endonuclease (e.g., ligand-inducible activation) by at least 30%, at least 40%, at least 50%, at least 60%, at least 70%, at least 80%, at least 90%, or at least 100%. In some embodiments, the binding of the stabilizing ligand to the destabilization domain increases activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds.

In some embodiments, at least one proteolytic cleavage site is incorporated into a Acr polypeptide that counteract the activity of a Cas-endonuclease, wherein cleavage of the at least one proteolytic cleavage site decreases the activity of the Cas-endonuclease (e.g., ligand-inducible inhibition) by at least 30%, at least 40%, at least 50%, at least 60%, at least 70%, at least 80%, at least 90%, or at least 100%. In some embodiments, the binding of the stabilizing ligand to the destabilization domain decreases the activity of the DD-Cas-endonuclease fusion by at least one fold, at least 2 folds, at least 5 folds, at least 10 folds, at least 20 folds, at least 30 folds, at least 40 folds, at least 50 folds, at least 60 folds, at least 70 folds, at least 80 folds, at least 90 folds, or at least 100 folds

In some embodiments, the Acr polypeptide can be further linked to one or more additional domains such as, e.g. a nuclear localization signal (NLS), a G-protein-coupled receptor (GPCR), a Gly-Ser linker, and a synthetic Notch receptor. As described below in Example 6 below, GPCR activation by cognate ligand binding induces recruitment of β-arrestin, which allows for release of dCas9-VPR for nuclear localization and gene activation. According, the activity and/or expression of the Acr polypeptide can be further modulated via incorporation of a GPCR polypeptide to create an integrated doxycycline-inducible CRISPRa or CRISPRi system.

As discussed above, the nucleic acids encoding the Acr polypeptide and/or the CRISPR system can be stably integrated in the host genome, or can be episomally replicating, or can be present in the engineered cell as a mini-circle expression vector for transient expression. Accordingly, in some embodiments disclosed herein, the nucleic acids are maintained and replicated in the engineered cell as an episomal unit, and the Acr polypeptide and/or one or more components of the CRISPR system are expressed episomally in the cell. In some other embodiments, the nucleic acids are stably integrated into the genome of the recombinant cell, wherein the Acr polypeptide and/or the one or more components of the CRISPR system are expressed from nucleic acids stably integrated in the genome of the cell.

In some embodiments, the expression and/or activity of the Acr polypeptide and/or the Cas endonuclease can be modulated at transcriptional level. In some embodiments, the expression of the Acr polypeptide and/or the one or more components of the CRISPR system is under control of a constitutive promoter. In principle, there are no particular limitations to the constitutive promoters that can be used. Non-limiting examples of suitable constitutive promoters include SV40 promoters, CMV promoters, PGK promoters, ubiquitin C (UBC) promoters, EF1A promoters, CAGG promoters, and a SFFV promoter. In some embodiments, the constitutive promoter is a CMV constitutive promoter. In some embodiments, the constitutive promoter is a PGK constitutive promoter.

In some embodiments, the expression of the Acr polypeptide and/or the one or more components of the CRISPR system is under control of a repressible promoter or an inducible promoter. In principle, the inducible promoter can generally be any the inducible promoter and can be, for example, a TRE3G inducible promoter, a tetracycline-regulated promoter, a steroid-regulated promoter, a metal-regulated promoter, an estrogen receptor-regulated promoter, or an inducible UAS promoter. In some embodiments, the inducible promoter is a TRE3G inducible promoter. In some embodiments, the inducible promoter is a tetracycline inducible promoter.

In some embodiments, the expression of the Acr polypeptide and/or the one or more components of the CRISPR system is under control of the same promoter.

In some embodiments, the engineered cell of the disclosure includes: (a) a first nucleic acid molecule encoding (i) an Acr polypeptide and (ii) a sgRNA; and (b) a second nucleic acid molecule encoding a nuclease-deficient Cas9 polypeptide (dCas9) fused to an effector domain, wherein the Acr polypeptide inhibits the activity of the dCas9-effector domain fusion and wherein the expression of the Acr polypeptide and the CRISPRa system is under control of the same promoter. In some embodiments, the first nucleic acid molecule encoding the Acr polypeptide and the sgRNA is stably integrated into the genome of the cell. In some embodiments, the first nucleic acid molecule the first nucleic acid molecule encoding the Acr polypeptide and the sgRNA is maintained episomally in the cell.

### Methods of Making Genetic Circuits

In another aspect, also provided herein are various methods for making a genetic circuit, including: (a) providing a plurality of first nucleic acid molecules each encoding a nuclease-deficient Cas endonuclease (dCas) fused to an effector domain; and (b) introducing each of the first nucleic acid molecules into a host cell comprising a second nucleic acid molecule to produce a plurality of engineered cells, wherein the second nucleic acid molecule encodes (i) an Acr polypeptide and (ii) a gRNA capable of directing the dCas9-effector domain fusion to a target gene in the engineered cells, and wherein the Acr polypeptide simultaneously inhibits the activity of the dCas-effector domain fusions expressed from the plurality of first nucleic acid molecules. In some embodiments, the dCas endonuclease is a dCas9 polypeptide. In some embodiments, the dCas endonuclease is a dCas12 polypeptide. In some embodiments, the dCas endonuclease comprises two non-contiguous portions (e.g., A and B) which, when combined, forms an active dCas. In some embodiments, the Acr polypeptide (e.g., C) inhibits the activity of the active dCas when both non-contiguous portions of dCas are present in the cell. In these instances, the three polypeptides A, B, and C constitute a logic gate, where both portions of dCas must be expressed and Acr polypeptide is absent in order to obtain output of the device (A AND B AND NOT C) (see, e.g., Example 12).

In some embodiments, the first nucleic acid molecule is stably integrated into the genome of the cell. In some embodiments, the second nucleic acid molecule is stably integrated into the genome of the cell. In some embodiments, both the first nucleic acid molecule and/or the second nucleic acid molecule are stably integrated into the genome of the cell. In some embodiments, the first nucleic acid molecule is maintained episomally in the cell. In some embodiments, the second nucleic acid molecule is maintained episomally in the cell. In some embodiments, both the first nucleic acid molecule and/or the second nucleic acid molecule are maintained episomally in the cell. In some embodiments, the target gene is an endogenous gene of the cell. In some embodiments, the first nucleic acid molecule is stably integrated into the genome of the cell and the second nucleic acid molecule is maintained episomally in the cell. In some embodiments, the first nucleic acid molecule is maintained episomally in the cell and the second nucleic acid molecule is stably integrated into the genome of the cell. In some embodiments, the target gene is an endogenous gene of the cell. In some embodiments, the target gene is an endogenous gene in the host cell. In some embodiments, the target gene is a reporter gene. In principle, there are no particular limitations to the reporter gene. Suitable reporter genes that can be used for the methods and compositions as described herein include, but are not limited to GFP, BFP, YFP, luciferase, and mCherry. In some embodiments, the effector domain is an activation domain. In some embodiments, the reporter gene is GFP. In some embodiments, the expression of the target gene expression is monitored by live-cell time-lapse microscopy. In a related aspect, provided herein is a genetic circuit that is produced by a method according to a method for making a genetic circuit as disclosed herein.

The methods disclosed herein may be deployed for selectively modulating CRISPR systems in cells of any species, including, but not limited to, prokaryotic and eukaryotic species. Suitable host cells to be engineered according to the present disclosure can include, but not limited to, algal cells, bacterial cells, fungal cells, plant cells, and animal cells. In some embodiments, the cell is an animal cell. In some embodiments, the animal cell is an invertebrate animal cell. In some embodiments, the vertebrate animal cell is a mammalians cell. In some embodiments, the mammalian cell is a human cell. In some embodiments, the human cell is a stem cell. In some embodiments, the stem cell is a human-induced pluripotent stem cell (hiPSC).

### Methods of Treatments

The engineered cells, compositions, kits, and methods of the disclosure can be used to treat individuals who have, who are suspected of having, or who may be at high risk for developing one or more health conditions or disorders. Exemplary health conditions and disorders of interest can include, without limitation, those associated with acute and chronic infections, inflammatory diseases, immune diseases, and various cancers.

In one aspect, provided herein are various methods of treating a disorder or health condition in a subject, the method including providing to a cell in the subject: (a) an anti-CRISPR (Acr) polypeptide or nucleic acid encoding the Acr polypeptide; and (b) a CRISPR-based system or nucleic acid encoding the system, wherein the Acr polypeptide modulates an activity of the CRISPR-based system in said cell.

For example, the experiments described in the Examples below were performed to characterize and evaluate a panel of natural phage-derived anti-CRISPR proteins using both CRISPRi and CRISPRa in mammalian cells (see, e.g., Examples 3-4). In particular, it is demonstrated that Arc proteins, exemplified by AcrIIA4, are a potent regulators of (d) Cas9 activity in a wide variety of contexts (e.g., reporter genes or endogenous genes) and different cell types such as, e.g., human embryonic kidney cells (HEK293T), human-induced pluripotent cells (hiPSC), and yeast cells. The small size of AcrIIA4 allows it to be incorporated in a range of contexts easily, and it remains highly efficient in inhibiting CRISPR activity when fused to other gene products via many linkers including 2A peptides at either the N- or C-terminus, or larger domains such as DD or fluorescent proteins. Experimental results presented herein also demonstrated that Arc proteins, e.g., AcrIIA4, are effective in ablating CRISPR-based complex synthetic devices. For example, fusing a destabilization domain to AcrIIA4 enabled tunable and inducible control of CRISPRa activity (see, e.g., Example 10). The data presented herein contributes to a larger picture on the use of Acrs as an additional layer of control over (d) Cas9 activity in eukaryotic cells. Thus, the present disclosure expands the existing CRISPRa/CRISPRi tool set by characterizing a useful and tunable inhibitor molecule, which is useful for probing biology in a wider range of contexts.

It was also observed that AcrIIA4 could operate when present at low-copy number (e.g., genomically integrated versus transiently transfected), and there was no apparent cytotoxic effect nor loss of expression of Acr over months of culturing in HEK293T and hiPSC lines. AcrIIA4 inhibits Spy (d) Cas9, by far the most commonly used CRISPR system, but may potentially be bypassed with another Cas9 ortholog. However, due to the small size of most Acrs, it is also contemplate that one can create a cassette encoding multiple Acrs targeting commonly used orthologs, especially as the wealth of Acrs becomes uncovered or to use promiscuous anti-CRISPRs as broad-spectrum inhibitors.

Based on the results described herein, it was contemplated that an integration approach will also be useful for defending against Cas9-based editing in various eukaryotic cells, such as S. cerevisiae; which in turn can be useful to preserve the integrity of cell lines used in the production of sensitive materials including toxic products and controlled substances. Additionally, it is contemplated that genomically write-protected organisms can be used as a safety valve for counteracting CRISPR-based gene drives. This is because Cas9-driven gene drives have previously been implemented in organisms such as mosquitos but one major concern has been the potential for unforeseen ecological impacts resulting from population collapse. Hence, without being bound to any particular theory, it is also contemplated that the introduction of a population with an integrated Acr will inhibit and possibly allow for the reversal of the spread of the gene drive. In fact, the results of a proof-of-principle study demonstrated the use of Acrs for the inhibition of a gene drive in yeast (see, e.g., Example 5).

As demonstrated in the Examples below, the use of CRISPRa and CRISPRi for assessing Acr activity relative to editing assays affords several advantages. For example, the assays are quick and can provide quantitative information on Acr activity (rather than a discrete readout of edited/not-edited) on the single-cell level, and thus are ideal for assessing Acr activity in mammalian cells. Further, the combination of CRISPRa and CRISPRi assays, in addition to editing assays, allows for distinguishing between specific anti-CRISPR activity and potential cytotoxic effects. The experimental results described herein indicate that one can begin to move from a regime of classifying Class 2 Cas targeting Acrs in a binary manner (i.e., as effective or non-effective) into one where anti-CRISPR activity can be more finely determined. It is further contemplated that these assays, coupled with the feasibility of using such organisms as E. coli and S. cerevisiae, allow for rapidly screening of discovered or designed Acrs for enhanced or tuned activity, activity targeting Cas9 variants and orthologs, and promiscuity of Acr activity. Based on the mechanism of Cas9, novel Acrs that can modulate dCas9 activity can also prove useful for inhibiting Cas9-driven editing.

As further demonstrated below, the use of Acrs in regulating CRISPR activity allows for the generation of more advanced dynamic control over gene regulation. In particular, the experimental data presented herein demonstrate that incorporating inducible control over Arcs, e.g., AcrIIA4 can be superior to using the same technology directly on Cas9. Without being bound to any particular theory, it is believed that the temporal dynamics of Acrs under such methods of control would be sharper compared to direct control over Cas9, because Acr is smaller and can be likely produced and degraded more quickly. The level at which Acrs operate is distinct from other methods of inducible control, allowing for multilayered logical control over dCas9-based gene regulation. A recent report also demonstrated the utility of incorporating control on the Acr by realizing light-dependent activity of Acr, further cementing this notion of Acr as an easily adaptable mode of control over dCas9 function.

The experimental data described in the Examples below demonstrates a proof-of-concept pulse generator circuit implemented via Acr-dCas9 interaction. While a similar type of circuit was previously reported in bacteria, a synthetic pulse response circuit has not been reported in mammalian cells. Utility of these circuits can potentially be enhanced by adding additional nodes of control (such as dCas9 orthologs/Acrs or other gene regulation tools) and implementing control over endogenous genes, as well as computational modeling to analyze and optimize circuit performance. The results described herein open the door to use dCas9 and Acrs to build dynamic pre-programmed gene regulation circuits. With further investigation, Acrs are poised to enter the realm of quantitative synthetic biology, potentially integrating multiple inputs and dCas9 effectors into genetically encoded gene regulation programs.

### Kits

Some embodiments of the disclosure contemplate kits for the practice of the methods disclosed herein. In some embodiments, the kits includes one or more of the above-described compositions, e.g., Cas endonucleases of a CRISPR system, anti-CRISPR (Acr) polypeptides, gRNAs, donor templates, and engineered cells.

In some embodiments, the kits include (a) a Cas endonuclease of a CRISPR system or a first nucleic acid encoding the Cas endonuclease; and (b) an anti-CRISPR (Acr) polypeptide or a second nucleic acid encoding the Acr polypeptide, wherein the Acr polypeptide modulates an activity of the CRISPR system in an inducible and/or programmable manner. In some embodiments, the kits include a first nucleic acid encoding a nuclease-deficient Cas9 polypeptide fused with an effector domain and a second nucleic acid encoding at least one sgRNA gene.

In some embodiments, the kit further includes a sequence encoding at least one gRNA and/or a sequence encoding a donor template encoding a gene-of-interest (GOI). In some embodiments, the sequence encoding the gRNA and/or the sequence encoding a donor template is incorporated in the first nucleic acid. In some embodiments, the sequence encoding the gRNA and/or the sequence encoding a donor template is incorporated in the second nucleic acid.

Non-limiting exemplary embodiments of the disclosed kit of the disclosure include one or more of the following features. In some embodiments, the kit further includes a sequence encoding at least one gRNA and/or a sequence encoding a donor template including a nucleic acid sequence encoding a gene-of-interest (GOI). In some embodiments, the sequence encoding the gRNA is incorporated in the first nucleic acid or in the second nucleic acid. In some embodiments, the sequence encoding the GOI is incorporated in the first nucleic acid or in the second nucleic acid.

In some embodiments, a kit can further include instructions for using the components of the kit to practice the methods described herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or subpackaging), etc. The instructions can be present as an electronic storage data file present on a suitable computer readable storage medium, e.g., CD-ROM, diskette, flash drive, etc. In some embodiments, the actual instructions are not present in the kit, but means for obtaining the instructions from a remote source (e.g., via the Internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference. As appropriate, procedures involving the use of commercially available kits and reagents are generally carried out in accordance with manufacturer defined protocols and/or parameters unless otherwise noted.

Additional embodiments are disclosed in further detail in the following examples, which are provided by way of illustration and are not in any way intended to limit the scope of this disclosure or the claims.

### Example 1

### General Experimental Procedures

### Materials

Plasmids and cell lines were generated using standard molecular cloning techniques. See Tables 1˜4 below for details on constructs used.

TABLE 1
Cell lines.

| Cell line | Construct/Genotype | Reference |
|---|---|---|
| HEK293T | wild-type | Clontech |
| HEK293T | TRE3G → dscGFP | This study |
| HEK293T | SV40 → eGFP | (Gao, et al., 2016) |
| HEK293T | EF1α → rtTA; TRE3G → KRAB-dCas9-HA-NLS-NLS-P2A- | This study |
| | sfGFP | |
| HEK293T | EF1α → rtTA; TRE3G → dCas9-HA-NLS-NLS-VPR-P2A- | This study |
| | sfGFP | |
| hiPSC | EF1α → rtTA; TRE3G → dCas9-HA-NLS-NLS-VPR-P2A- | This study |
| | sfGFP | |
| CEN.PK2-1D | MATα ura3-52; trp1-289; leu2-3, 112; his3Δ1; MAL2-8C; | (Entian and Kötter, 2007) |
| | SUC2 | |
| yJZC10 | MATa leu2-3, 112 trp1-1 can1-100 ura3-1 his3-11, 15 | (Zalatan, et al., 2014) |
| | HO::rtTA-HygB TRP1::pTETO7-Venus LEU2::pTDH3- | |
| | dCas9-3XNLS HIS3::pAdh-MCP-VP64 | |
| yJZC14 | MATa leu2-3, 112 trp1-1 can1-100 ura3-1 his3-11, 15 | Unpublished |
| | HO::rtTA-HygB TRP1::pTETO7-Venus LEU2::pTDH3- | |
| | dCas9-3XNLS mfa2:pTEF1-mCherry-kanR | |
| HEK293T | EF1α → mCherry-P2A-AcrIIA4 | This study |
| HEK293T | mU6 → sgTET; CMV → mCherry | This study |
| HEK293T | mU6 → sgTET; CMV → mCherry-P2A-AcrIIA4 | This study |
| HEK293T | mU6 → sgTET; TRE3G → mCherry-P2A-AcrIIA4 | This study |

TABLE 2
Cas9 plasmids.

| Construct | Reference |
|---|---|
| PGK → KRAB-BFP-dCas9-HA-NLSx2 | (Gao, et al., 2016) |
| PGK → VPR-BFP-dCas9-HA-NLSx2 | (Gao, et al., 2016) |
| pRNR2 → Cas9-NLS-His6 | This study |
| mU6 → sgPD1-ko; TRE3G → NLS-Cas9-NLS-eGFP | This study |
| CMV → dCas9-HA-VPR-mCherry | (Kipniss, et al., 2017) |
| CMV → NES-ARRB2-NES-TCS-dCas9-HA-VPR-mCherry | (Kipniss, et al., 2017) |
| PGK → DD-VPR-BFP-dCas9-HA-NLSx2-T2A-BFP | This study |
| PGK → VPR-BFP-dCas9-HA-NLSx2-P2A-AcrIIA4 | This study |

TABLE 3
sgRNA sequences.

| sgRNA | Sequence | SEQ ID NO |
|---|---|---|
| sgTET | GTACGTTCTCTATCACTGATAGTTTAAGAGCTATGCTGGAA | 1 |
| | ACAGCATAGCAAGTTTAAATAAGGCTAGTCCGTTATCAACT | |
| | TGAAAAAGTGGCACCGAGTCGGTGCTTTTTTT | |
| sgSV40 | GAAAGTCCCCAGGCTCCCCAGCGTTTAAGAGCTATGCTGGA | 2 |
| | AACAGCATAGCAAGTTTAAATAAGGCTAGTCCGTTATCAAC | |
| | TTGAAAAAGTGGCACCGAGTCGGTGCTTTTTTT | |
| sgmRFP_RR1 | GAACTTTCAGTTTAGCGGTCTGTTTAAGAGCTATGCTGGAA | 3 |
| | ACAGCATAGCAAGTTTAAATAAGGCTAGTCCGTTATCAACT | |
| | TGAAAAAGTGGCACCGAGTCGGTGCTTTTTTT | |
| sgCXCR4-1a | GCCTCTGGGAGGTCCTGTCCGGCTCGTTTAAGAGCTATGCTG | 4 |
| | GAAACAGCATAGCAAGTTTAAATAAGGCTAGTCCGTTATCA | |
| | ACTTGAAAAAGTGGCACCGAGTCGGTGCTTTTTTT | |
| sgCXCR4-3a | GCAGACGCGAGGAAGGAGGGCGCGTTTAAGAGCTATGCTGG | 5 |
| | AAACAGCATAGCAAGTTTAAATAAGGCTAGTCCGTTATCAA | |
| | CTTGAAAAAGTGGCACCGAGTCGGTGCTTTTTTT | |
| sgCXCR4-20i | gcagaagcggccaggacattggGTTTAAGAGCTATGCTGGAAACAGCA | 6 |
| | TAGCAAGTTTAAATAAGGCTAGTCCGTTATCAACTTGAAAA | |
| | AGTGGCACCGAGTCGGTGCTTTTTTT | |
| sgTRP1 | GGCCGGCATGGTCCCAGCCTCCTCGCTGGCGCCGGCTGGGC | 7 |
| | AACACCTTCGGGTGGCGAATGGGACTTTccggatcaagattgtacgtaG | |
| | TTTTAGAGCTAGAAATAGCAAGTTAAAATAAGGCTAGTCCG | |
| | TTATCAACTTGAAAAAGTGGCACCGAGTCGGTGCTTTT | |
| sgTET0-2x | ACTTTTCTCTATCACTGATAGTTTTAGAGCTAGAAATAGCAA | 8 |
| (wt + f6) | GTTAAAATAAGGCTAGTCCGTTATCAACTTGAAAAAGTGGC | |
| MS2 | ACCGAGTCGGTGCGGGAGCACATGAGGATCACCCATGTGCG | |
| | ACTCCCACAGTCACTGGGGAGTCTTCCC | |
| sgGAL4 | gagcactgtcctccgaacgtGTTTAAGAGCTAAGCTGGAAACAGCATA | 9 |
| | GCAAGTTTAAATAAGGCTAGTCCGTTATCAACTTGAAAAAG | |
| | TGGCACCGAGTCGGTGCTTTTTTT | |
| sg-dGFP | GGTGGCGGATCAGAAGGAGGgtttaagagctatgctggaaacagcatagcaag | 10 |
| | tttaaataaggctagtccgttatcaacttgaaaaagtggcaccgagtcggtgcttttttt | |
| sgPD1-ko | ggaagcggcagtcctggcgtttAagagctaagctggaaacagcatagcaagttTaaataaggct | 11 |
| | agtccgttatcaacttgaaaaagtggcaccgagtcggtgcttttttt | |
| sgRELA | gatctccacataggggccag | 12 |
| sgCDC42BPB | gagccgcaccttggccgaca | 13 |

sgRNA sequences used in experiments: spacer sequence; HDV ribozyme, MS2.

For reporter experiments: sgSV40 was used as the negative control sgRNA for the TRE3G promoter, and sgTET was used as the negative control sgRNA for the SV40 promoter.

For CXCR4 experiments: sgCXCR4-1a and sgCXCR4-3a were used in combination for HEK293T activation; sgCXCR4-20i was used for repression; sgCXCR4-3a was used for hiPSC activation; sgmRFP RR1 was used as the negative control sgRNA.

For genome editing reporter experiments, sgGAL4 was used as non-targeting guide and sg-dGFP for on-target guide. sgPD1-ko as above was used for WPC gene knock-out plasmid transfections. sgPD1-ko, sgRELA, and sgCDCl42BPB spacer sequences were used with a modified scaffold sequence for RNP delivery.

TABLE 4
Acr sequences.

| Protein | | |
|---|---|---|
| domain | Sequence | SEQ ID NO |
| AcrIIC1 | MAKEVFKLKPELVTYKGCGWALACIKDGEIIDLTYVRDLGIEEY | 14 |
| | DENFDGLEPEIIYYDVVASQACKEVAYRYEEMGEFTFGLCSCWE | |
| | FNVM | |
| AcrIIC3 | MAFKRAIIFTSFNGFEKVSRTEKRRLAKIINARVSIIDEYLRAKDTN | 15 |
| | ASLDGQYRAFLFNDESPAMTEFLAKLKAFAESCTGISIDAWEIEES | |
| | EYVRLPVERRDFLAAANGKEIFKI | |
| AcrIIA1 | MTIKLLDEFLKKHDLTRYQLSKLTGISQNTLKDQNEKPLNKYTVS | 16 |
| | ILRSLSLISGLSVSDVLFELEDIEKNSDDLAGFKHLLDKYKLSFPA | |
| | QEFELYCLIKEFESANIEVLPFTFNRFENEEHVNIKKDVCKALENA | |
| | ITVLKEKKNELL | |
| AcrIIA2 | MTLTRAQKKYAEAMHEFINMVDDFEESTPDFAKEVLHDSDYVVI | 17 |
| | TKNEKYAVALCSLSTDECEYDTNLYLDEKLVDYSTVDVNGVTY | |
| | YINIVETNDIDDLEIATDEDEMKSGNQEIILKSELK | |
| AcrIIA3 | MTKYNKSEIMKNAWAMFNSYEWDVENFKFVSAENKTFSNCLKE | 18 |
| | AWAEEKEYVERKAKETAEAPRSEEAKAWDWACRKLNVNDLQN | |
| | IDATDKVFYVVDMQKEMWTSNVWAQAIKAVELYVKLGLA | |
| AcrIIA4 | MNINDLIREIKNKDYTVKLSGTDSNSITQLIIRVNNDGNEYVISES | 19 |
| | ENESIVEKFISAFKNGWNQEYEDEEEFYNDMQTITLKSELN | |
| AcrIIA5 v1 | MAYGKSRYNSYRKRNFSISDNQRREYAKKMKELEQAFENLDG | 20 |
| | WYLSSMKDSAYKDFGKYEIRLSNHSADNRYHDLENGRLIVNVK | |
| | ASKLNFVDIIENKLGKIIEKIDTLDLDKYRFINATKLERDIKCYYK | |
| | GYKTKKDVI | |
| AcrIIA5 v2 | MAYGKSRYNSYRKRSFNRSNKQRREYAQEMDRLEKAFENLDG | 21 |
| | WYLSSMKDSAYKDFGKYEIRLSNHSADNKYHDLENGRLIVNIK | |
| | ASKLNFVDIIENKLDKIIEKIDKLDLDKYRFINATNLEHDIKCYYK | |
| | GFKTKKEVI | |
| AcrIIA6 | MKINDDIKELILEYMSRYFKFENDFYKLPGIKFTDANWQKFKNG | 22 |
| | GTDIEKMGAARVNAMLSCLFEDFELAMIGKAQTNYYIDNSLKL | |
| | NMPFYAYYDMFKKQLLINWLKNNRDDVICGTGRMYTASGNYI | |
| | ANAYLEVALESSRLGGGEYMLQMRFKNYSRSQEPIPSGRQNRLE | |
| | WIENNLENIR | |

### Cell Cultures

HEK293T cells (Clontech) were cultured in DMEM+GlutaMAX (Thermo Fisher) supplemented with 10% Tet-FBS (Clontech). Human iPSCs were cultured in mTeSR (STEMCELL Technologies). Cells were maintained and passaged using standard cell culture techniques and were maintained at 37° C. and 5% CO2. Cells were not regularly monitored for mycoplasma contamination.

Transient transfections were performed using TranslT-LT1 transfection reagent (Mirus). Lentivirus for generating reporter cell lines and sgRNA transduction was packaged using wild-type HEK293T (Clontech). Lentiviral transduction was performed at roughly 0.25 multiplicity-of-infection. Transduced HEK293T cell lines were sorted in bulk (except for the write-protected cell line, which was clonally sorted). The cell line containing the IFFL was sorted for negative mCherry expression, then transfected with a plasmid bearing tetracycline-controlled transactivator (tTA) and sorted for positive expression 2 days post-transfection. Doxycycline-inducible KRAB-dCas9 and dCas9-VPR HEK293T stable lines were generated using the PiggyBac transposon system. dCas9-VPR and rtTA were knocked into the AAVS1 locus in hiPSCs using techniques as previously described; briefly, plasmids bearing TALENs targeting the AAVS1 locus were co-transfected along with plasmid containing the VPR-dCas9+rtTA construct using the Amaxa Nucleofection system and a clonal population isolated.

Activation experiments were assessed for activity 2 days post-transfection; repression experiments were assessed at 5 days. Doxycycline-inducible constructs were maintained in medium supplemented with 1/mL doxycycline starting from the transfection date (except for −dox conditions). For GPCR experiments, cells were exposed to 20 μM ligand clozapine-N-oxide for 1-2 days before assessing activity. For DD-Shield1 experiments, medium of cells was supplemented with appropriate amount of Shield1 ligand immediately following transfection.

### Gene Regulation and Flow Cytometry and Analysis

For CXCR4 expression analysis, cells were targeted with APC-labeled CXCR4 antibody (BioLegend #306510) before flow cytometry assays. Analysis of flow cytometry data, including compensation, was performed using FlowJo. Cells were gated for viability and single cells, as well as positive fluorescence markers. Fluorescence values were normalized within experimental runs to an untransfected control. For plotting, values were normalized to the non-targeting sgRNA negative control condition, except for hiPSC data, which were normalized to the +dox, no sgRNA condition. Unless otherwise stated, average values provided are geometric means and error bars are ±s.e.m. Significance tests and p-values were calculated using two-sided Welch's t-tests.

### Mammalian Editing Experiments

For gain-of-function editing experiments, a HEK293T reporter cell line with out-of-frame split-GFP construct was transfected with Cas9 and sgRNA plasmid with and without plasmid containing AcrIIA4. 3 days after transfection, cells were analyzed via flow cytometry without gating for presence of plasmid.

For write-protection editing experiments, HEK293T cells were transiently transfected with plasmid bearing Cas9-eGFP and sgRNA, as above, or Cas9 protein (IDT or Synthego) with synthetic modified sgRNA (Synthego) via electroporation (Invitrogen Neon or Amaxa Nucleofection). Plasmid transfections were sorted 1-2 days post-transfection for presence of GFP. Genomic DNA was isolated via spin-column chromatography (Qiagen), amplified at target locus via PCR (Kapa Biosystems), and assessed via T7E1 endonuclease activity (NEB) or TIDE sequencing analysis. Sequencing traces (Quintara Biosciences) were compared to untransfected conditions to determine editing efficiency; sequencing traces of untransfected conditions were compared to each other to determine baseline noise of the TIDE assay.

### Microscopy Data and Analysis

Cells for microscopy were cultured in FluoroBrite DMEM medium supplemented with GlutaMAX (Thermo Fisher) and 10% Tet-FBS. Cells were transfected as above, and immediately placed in a microscopy chamber pre-equilibrated and maintained at 37° C. and 5% CO2. Selected fields of view were automatically imaged for mCherry, GFP, and BFP fluorescence and combined into time series post-acquisition.

Movies were background-subtracted (ImageJ), and cell-centered traces were generated by an automated algorithm for computationally tracking spots in the GFP channel and joining identified spots into combined tracks (TrackMate). Spots were filtered by threshold and signal-to-noise ratio, and traces comprising fewer than 12 spots were automatically discarded. Cell-centered sub-movies were generated on remaining traces and analyzed via a custom Python script. To calculate fluorescence for each trace, a 31 pixel (˜10 μm) square region was isolated, and the arithmetic mean of the top 50% highest intensity pixels was calculated for each frame of the sub-movie. To perform the fit, these traces were fit to an asymmetric gaussian function using a trace-specific to (center of gaussian) and amplitude and global minimum value and rate constants governing decay to the left and right of the center of the curve. To account for spurious detection and low-signal traces, an iterative filtering process was applied, discarding any traces that had amplitude less than the minimum value (e.g., lower than 2-fold change) and then re-performing the fit until convergence.

### Yeast Experiments

For editing experiments, competent yeast stocks (CEN.PK2-1D strain) were generated and transformed with standard protocols (Frozen-EZ Yeast Transformation II Kit, Zymo Research) and were grown at 30° C. in yeast peptone dextrose (YPD) liquid medium supplemented with 80 mg/L adenine hemisulfate and YPD-agar plates with 1 g/L monosodium glutamate and 400 mg/L G418 sulfate. Transformants were recovered for 2 h in YPD before plating with G418 selection. Colony formation was assessed 48-60 h after plating.

For toxicity experiments, CEN.PK2-1D was transformed with Acr plasmids alone, as above, plating on synthetic complete agar plates lacking uracil. For CRISPRa and CRISPRi experiments, reporter strains yJZC10 and yJZC14, respectively, were transformed with plasmids, as toxicity experiments. Overnight cultures were back-diluted 1:4 in synthetic complete medium lacking uracil and assessed 4-6 h later via flow cytometry. Control conditions (strains lacking plasmid) were transformed and grown in media containing uracil.

### Example 2

### Supplemental Modeling

A set of simple kinetic models involving production and degradation of relevant species for each circuit condition was constructed in an attempt to qualitatively recapitulate and describe the observed experimental behavior.

### Model 1: CRISPRa Condition

In this condition, VPR-dCas9 (C) is expressed from a constitutive promoter, and the reporter (G) is driven in a VPR-dCas9-dependent fashion. Simplifying assumptions were made that sgRNA concentration is saturating and that production of GFP is proportional to VPR-dCas9 concentration. Further, all production processes in a single term were bunched up. Therefore, the following rate equations governs this model.

$$\frac{d C}{d t} = {k}_{1} - {k}_{2} C \\ \frac{d G}{d t} = {k}_{3} C - {k}_{4} G$$

### Model 2: IFFL Condition

An Acr species (A) was added, with production also driven in a VPR-dCas9-dependent fashion, and assume that Acr may bind to dCas9, irreversibly inactivating it. This results in the following model.

$$\frac{d C}{dt} = {k}_{1} - {k}_{2} C - {k}_{7} A C \\ \frac{d A}{dt} = {k}_{5} C - {k}_{6} A - {k}_{7} A C \\ \frac{d G}{dt} = {k}_{3} C - {k}_{4} G$$

### Model 3: Acr Condition

As Model 2, except Acr was produced constitutively.

$$\frac{d C}{dt} = {k}_{1} - {k}_{2} C - {k}_{7} A C \\ \frac{d A}{dt} = {k}_{8} - {k}_{6} A - {k}_{7} A C \\ \frac{d G}{dt} = {k}_{3} C - {k}_{4} G$$

Because the Acr is stably integrated, it was assumed that a steady-state initial concentration in the model described herein to be given by.

$${A}_{0} = \frac{{k}_{8}}{{k}_{6}}$$

Parameterization of Models and Sensitivity Analysis

Solutions for these equations were generated numerically (MATLAB). The models were parameterized by fitting to aligned cell-tracking traces (Model 1 and Model 2) and unaligned traces (Model 3). In order to narrow the explored parameter space, models were fitted to experimental data consecutively, propagating derived constants forward to subsequent models.

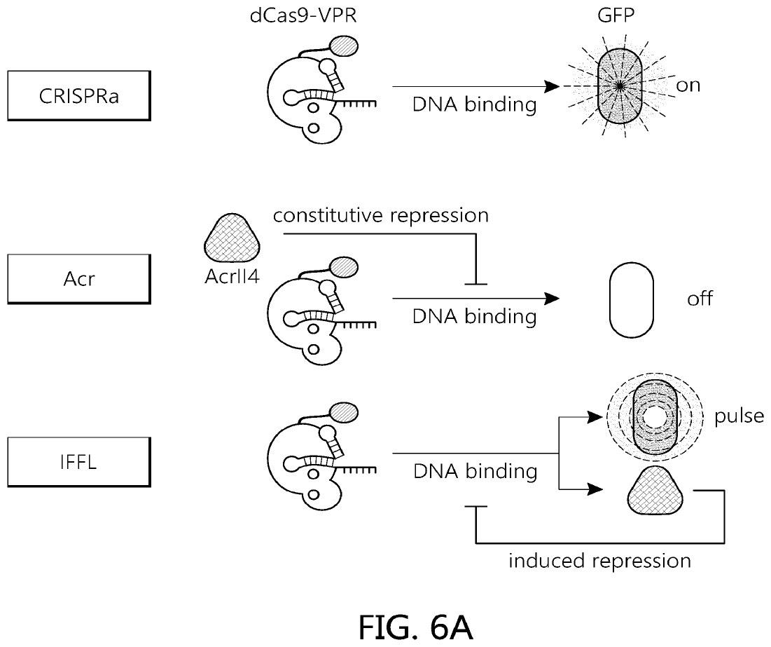

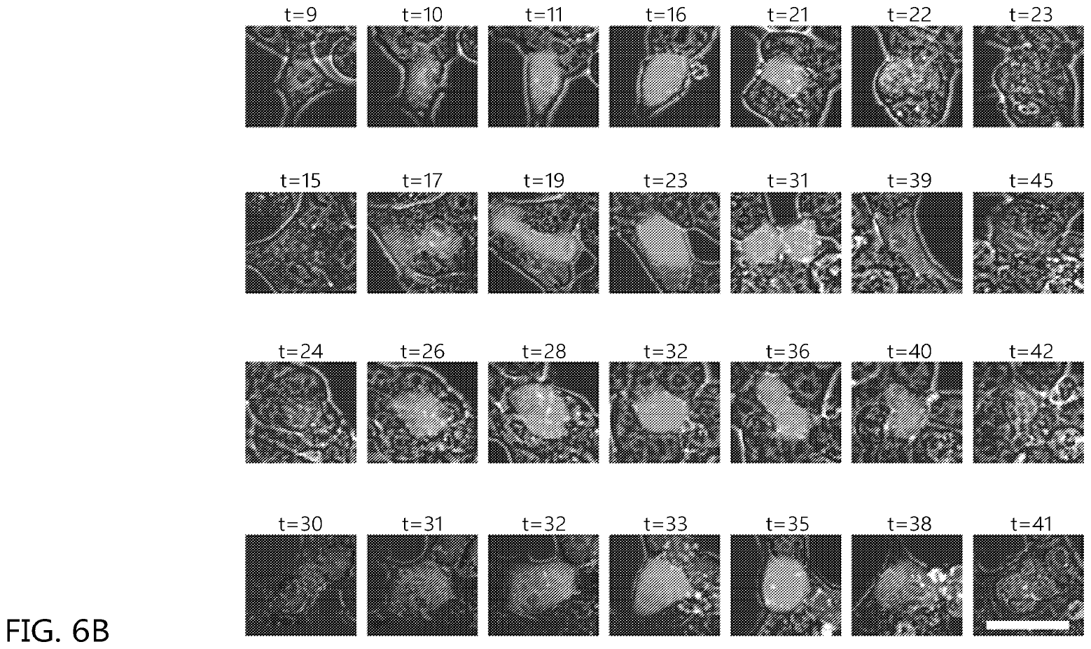

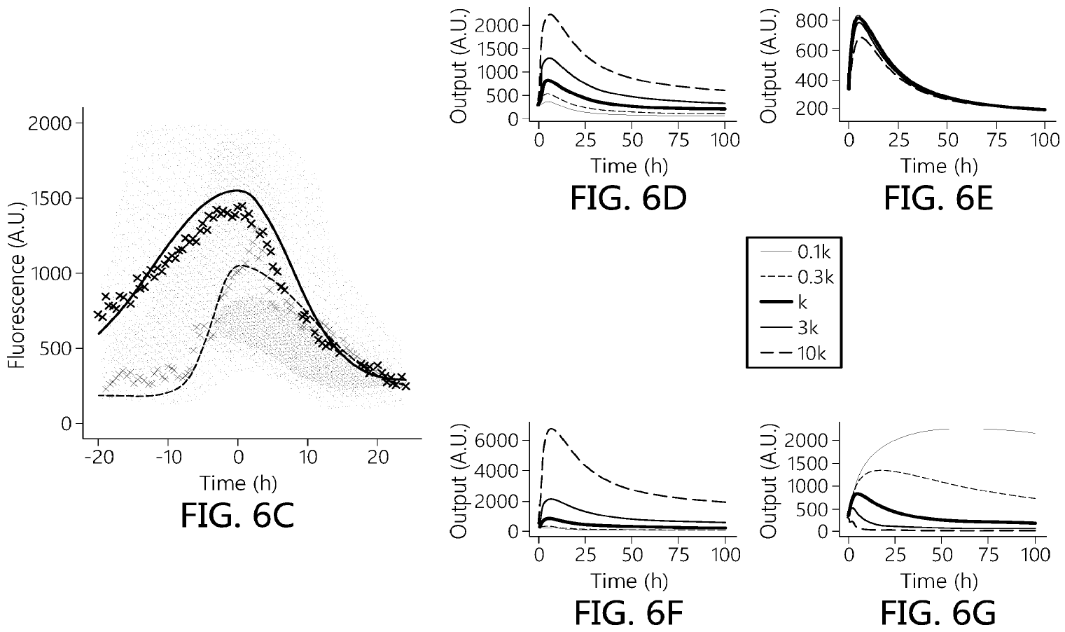

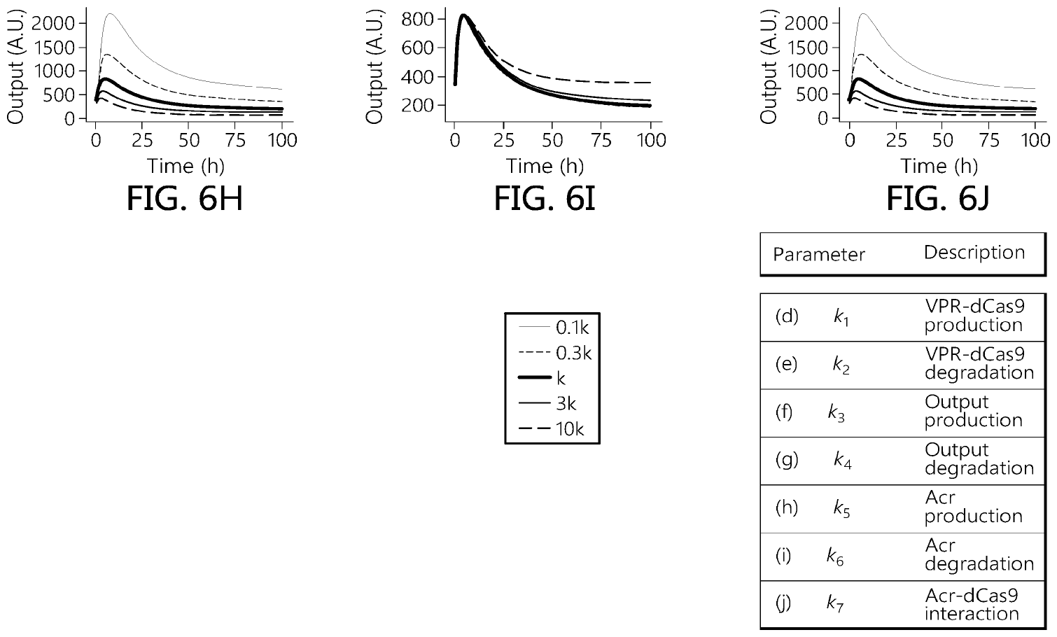

Based on the derived parameters from Model 2, each of the 7 rate constants were individually varied to generate computational predictions of circuit behavior in response to various perturbations in rate constants (shown in FIG. 6).

### Example 3

### Acrs Inhibit CRISPR-Based Gene Regulation in Mammalian Cells

This Example describe experiments performed to assess the efficacy of a panel of 5 Acrs (AcrIIC1, AcrIIA1, AcrIIA2, AcrIIA3, AcrIIA4) targeting Class 2 CRISPR systems to alter gene expression changes induced by CRISPRa and CRISPRi. As discussed above, CRISPR-based regulation of gene expression involves the use of a dCas9 with target sequence specified by a sgRNA. While DNA binding of dCas9 alone is sufficient for CRISPR-based gene interference (CRISPRi) in prokaryotes and yeast, optimal CRISPRi or CRISPR-based gene activation (CRISPRa) in most eukaryotic organisms involves the fusion of repressive or activating domains to dCas9, which, when targeted to a particular locus by an sgRNA, results in specific gene down- or up-regulation. It was therefore hypothesized that Acrs that function through the inhibition of Cas9 binding to DNA should be able to inhibit CRISPRa and CRISPRi (FIG. 1A), and, conversely, these gene regulation tools could be used to further characterize the function of Acrs.

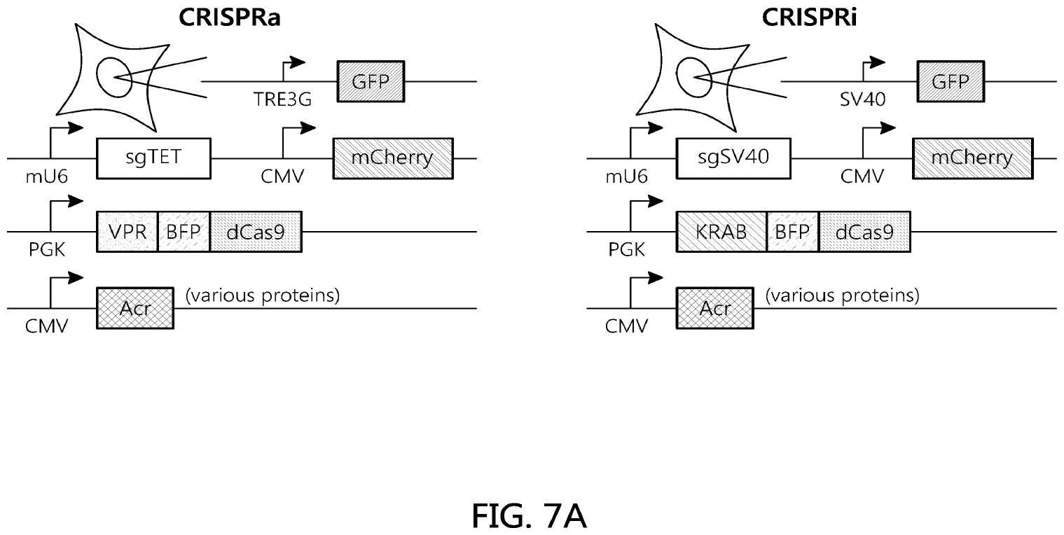

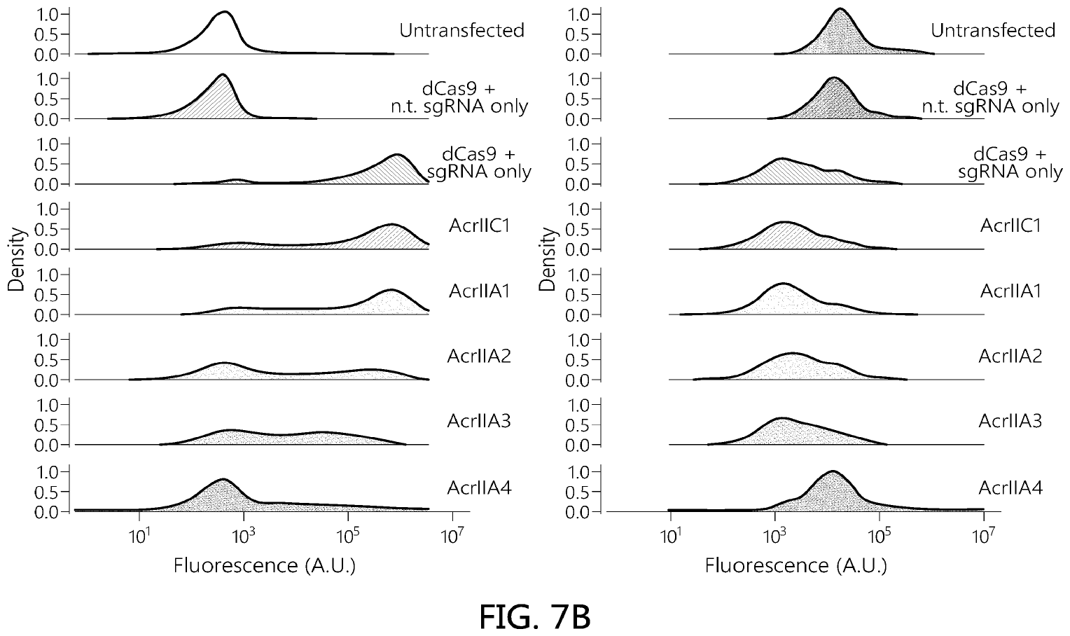

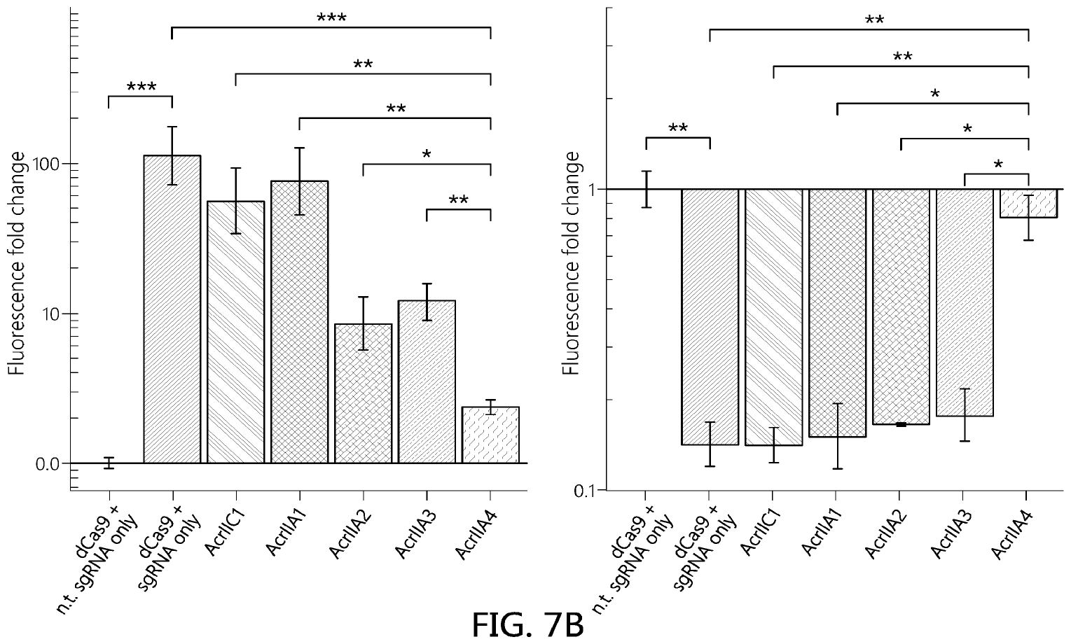

To investigate this hypothesis, a systematical assessment of the efficacy of a panel of 5 Acrs (AcrIIC1, AcrIIA1, AcrIIA2, AcrIIA3, AcrIIA4) targeting Type II CRISPR systems to alter gene expression changes induced by CRISPRa and CRISPRi was performed. Plasmids encoding dCas9-effectors, sgRNA, and Acrs were co-transfected into suitable HEK293T reporter cell lines (FIG. 7A). For CRISPRa, VPR-dCas9 was transfected into a reporter line bearing the inducible TRE3G promoter driving GFP expression; for CRISPRi, KRAB-dCas9 was used on a line with an SV40 promoter driving GFP. For both cell lines, the sgRNA sequence was designed to target the region of the promoter proximal to the transcription start site and cells were assessed for induced GFP reporter expression change.

Varying levels of inhibition of CRISPRa and CRISPRi caused by these Acrs were observed, with AcrIIA4 demonstrating consistently significant effect in negating CRISPR gene regulation (FIGS. 1B-1C), which is generally consistent with results involving CRISPRi in bacteria and editing in human cells, as well as the proposed mechanism of AcrIIA4 inhibiting DNA binding by serving as a DNA mimic.

Based on these results, the utility of the best-performing AcrIIA4 in controlling gene regulation was further explored. An optimized version of the described-above assay was designed, wherein the sgRNA and Acr were incorporated into a single plasmid by fusing the sgRNA plasmid's mCherry reporter to the Acr by a self-cleaving 2A peptide (FIG. 1B), using AcrIIC3 (demonstrated to be ineffective versus S. pyogenes Cas9) as a null Acr. In this assay, AcrIIA4 almost completely reduced gene up- or down-regulation to zero, whereas assays performed in the presence of AcrIIC3 showed similar levels of activity as control assays (FIGS. 1C-1E). These results demonstrated that this 2A fusion strategy can maintain Acr function and CRISPRa and CRISPRi can be used to quantitate Acr activity.

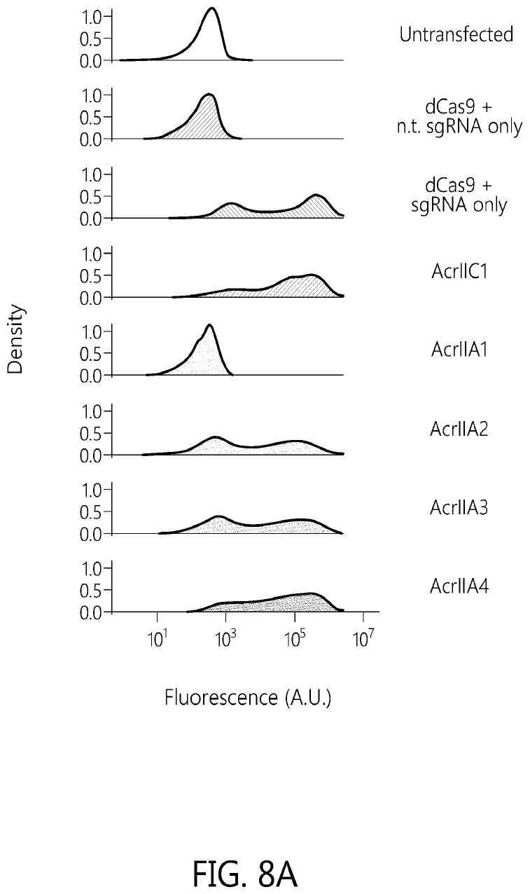

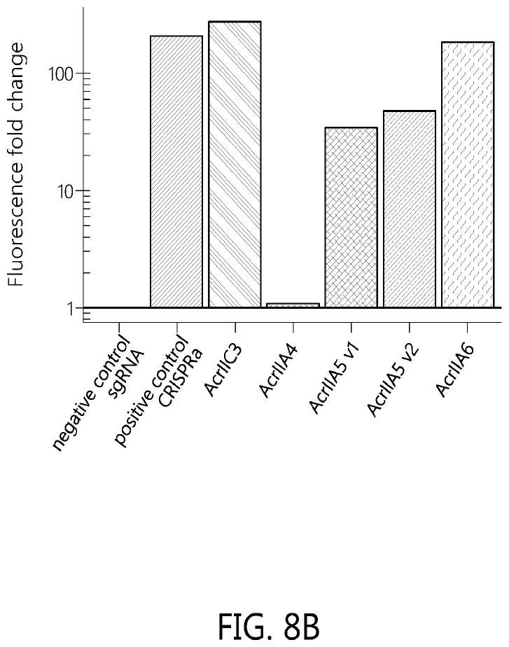

This 2A fusion strategy was subsequently used to characterize recently discovered II-A Acr families, AcrIIA5 and AcrIIA6 (see, e.g., FIGS. 8A-8B). By using this assay, it was determined that AcrIIA5 proteins demonstrated a modest (˜80%) reduction in dCas9-induced GFP expression, consistent with a previous report in yeast, whereas AcrIIA6 had no discernible effect.

### Example 4

### Acrs can Inhibit CRISPRa and CRISPRi on Endogenous Genes

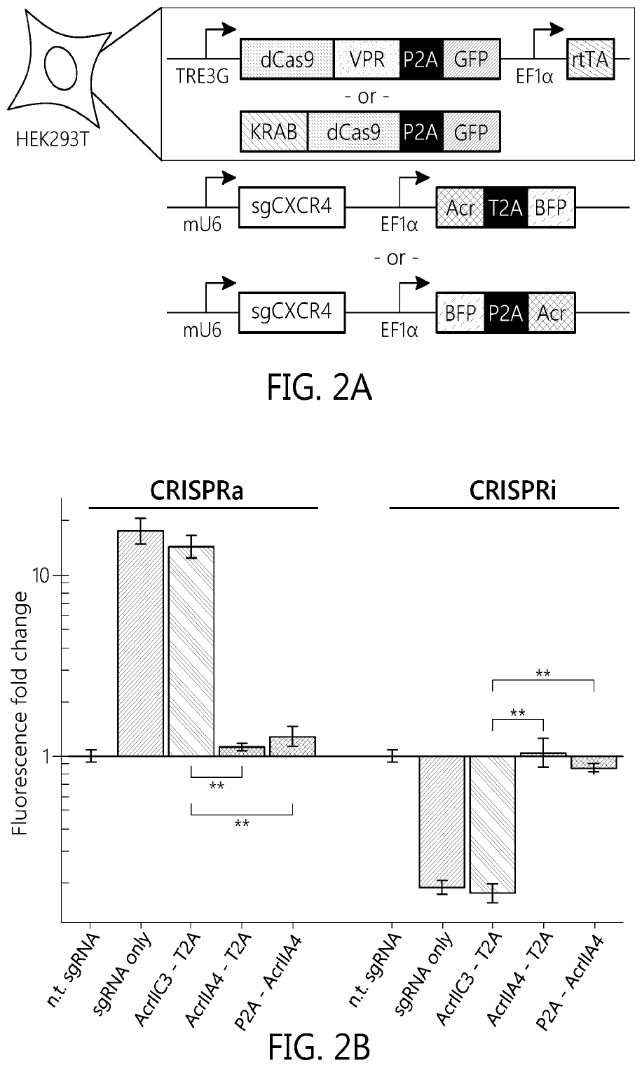

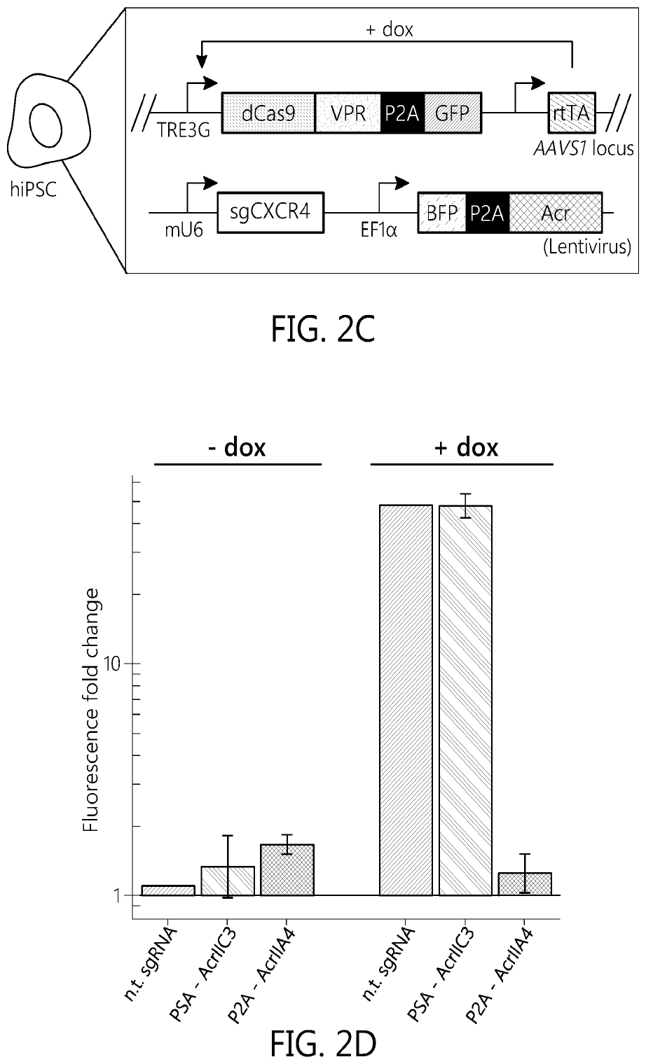

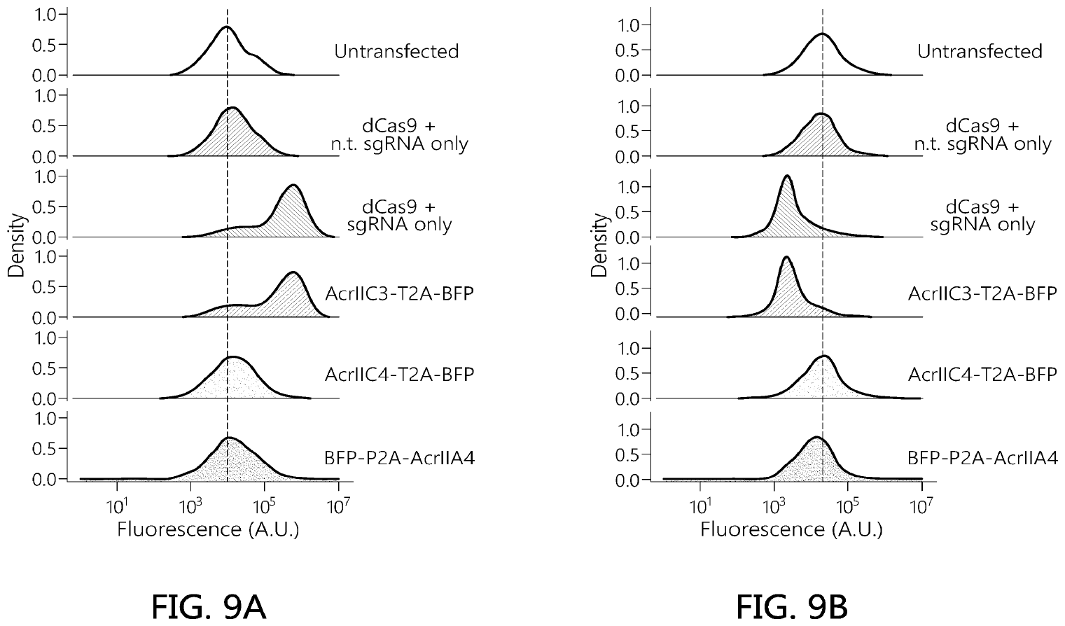

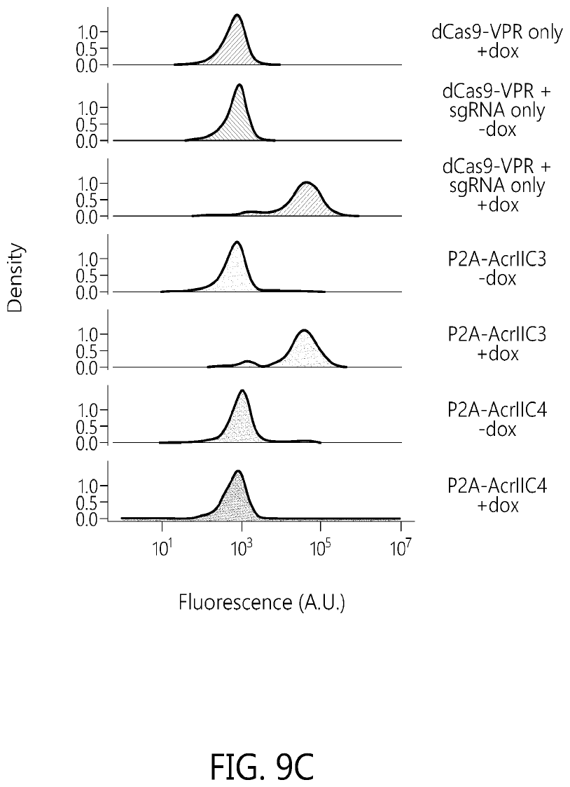

This Example describes experiments performed to investigate whether AcrIIA4 could be used in a wider variety of contexts, starting from endogenous gene regulation. A set of doxycycline (dox)-inducible CRISPRa and CRISPRi PiggyBac constructs was first created for integration into HEK293T (FIG. 2A). In these experiments, sgRNAs designed to target the expression of the endogenous C—X—C chemokine receptor type 4 (CXCR4) gene were used and strong CRISPRa (˜15-fold increase) and CRISPRi (˜85% decrease) activity was observed (FIGS. 2B and 9A-9B). By contrast, AcrIIA4 demonstrated almost total nullification of gene regulation activity, while AcrIIC3 showed little inhibitory effect (FIGS. 2A-2B and 9A-9B). It was observed that fusing AcrIIA4 N-terminal to the 2A peptide and fluorescent protein demonstrated slightly stronger anti-CRISPR effect, possibly due to differences in the coupling efficiency of the 2A peptide.

The applicability of AcrIIA4 in other cell types was subsequently tested. A human induced pluripotent stem cell (hiP SC) line was used with a dox-inducible CRISPRa construct knocked into the AAVS1 locus (FIG. 2C). This cell line was lentivirally transduced with constructs bearing CXCR4-targeting sgRNA with and without AcrIIC3 and AcrIIA4. Consistently, minimal effects on gene regulation with AcrIIC3 and strong inhibition of CRISPRa with AcrIIA4 were observed (FIGS. 2D and 9C). This suggests AcrIIA4 works for controlling CRISPR-based gene regulation in diverse mammalian cell types as a general tool.

### Example 5

### Acrs Inhibit CRISPR Activity in Yeast Cells

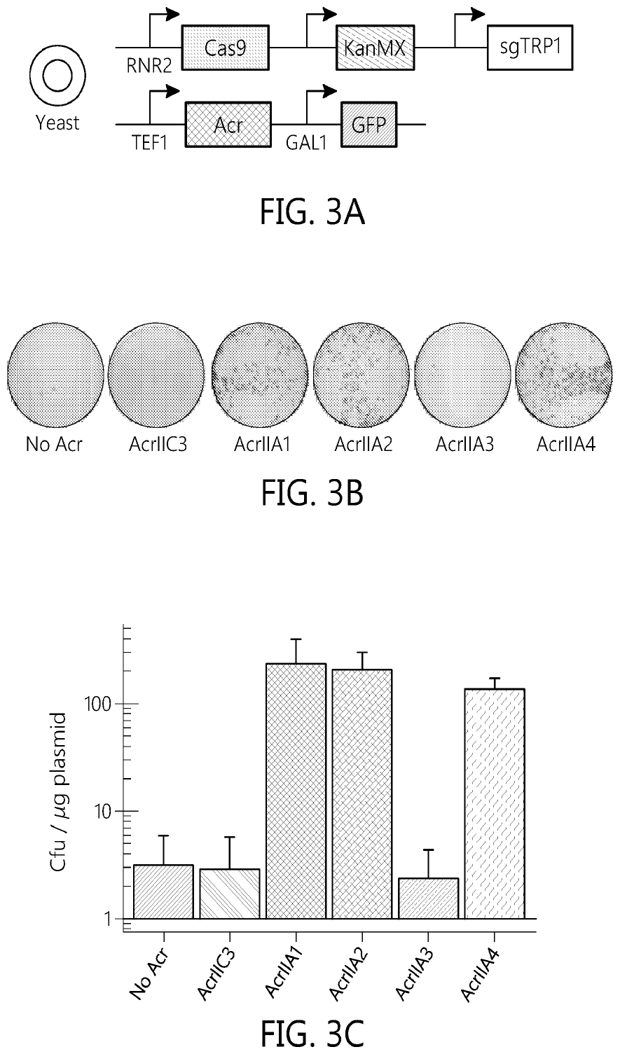

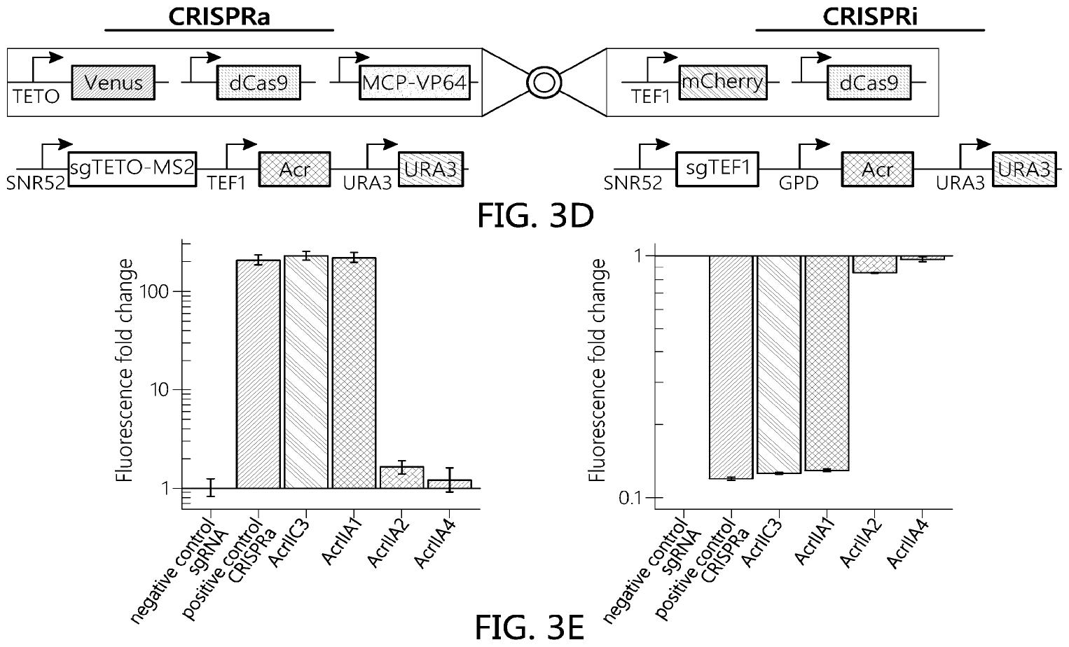

This Example describes experiments performed to investigate the efficacy of Acrs in other eukaryotic systems. S. cerevisiae is a common metabolic engineering platform and also provides a useful system for protein engineering. First, the activity of the Acr panel described herein using a yeast editing assay was assessed, in which yeast cells were co-transformed with a plasmid bearing Cas9, sgRNA targeting essential gene TRP1, and a KanMX selection marker alongside a plasmid bearing Acr genes (FIG. 3A). Cells could only survive on selection plates with active Acr inhibiting Cas9. Multiple Type IIA Acrs were found to be effective in abrogating CRISPR activity (FIGS. 3B-3C), including AcrIIA1, which showed minimal effect in the mammalian CRISPRa and CRISPRi assays described herein or in previously described CRISPRi and editing assays.

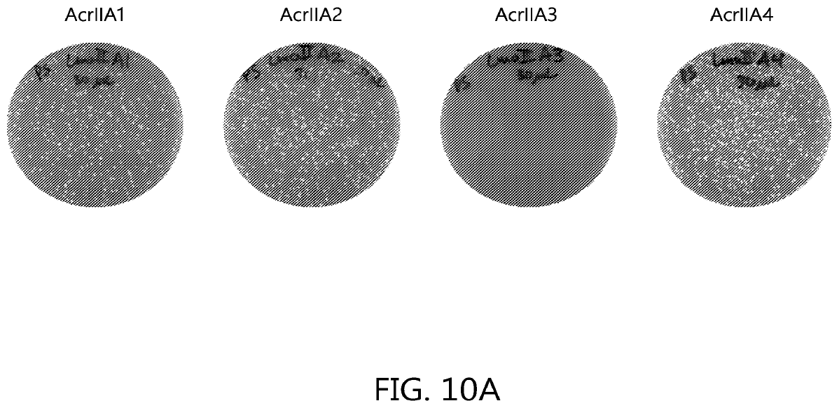

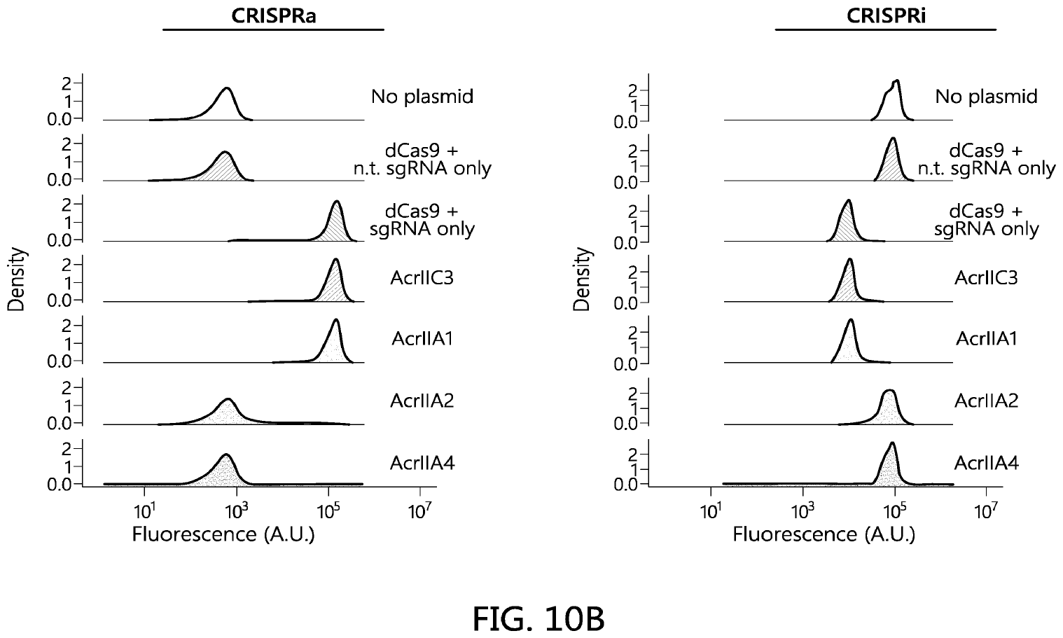

To further understand the function of Acrs in yeast, their activities on CRISPRa and CRISPRi were also tested using a pair of reporter strains and transforming a plasmid bearing sgRNA and Acr (FIG. 3D). Consistently, it was found that plasmids containing AcrIIA3 were toxic, even when transformed without dCas9 (FIG. 10A). AcrIIA3 was previously found to be toxic in bacteria, indicating that a common mechanism may underlie its biological effects. Other Acrs demonstrated a consistent effect on CRISPRa and CRISPRi (FIGS. 10B and 3E). Interestingly, all tested II-A Acrs were effective, except for AcrIIA1. These assays in concert suggest the following results: AcrIIA1 is a strong inhibitor of Cas9 editing in yeast but not dCas9-based gene regulation, indicating a possible mechanism of inhibition at the editing level (perhaps akin to a previously reported mechanism for a II-C Acr); and AcrIIA2 demonstrates stronger apparent activity for genome editing and gene regulation in yeast than in mammalian cells (consistent with other reports).

### Example 6

### Acrs Negate Activity of CRISPR-Based Synthetic Devices

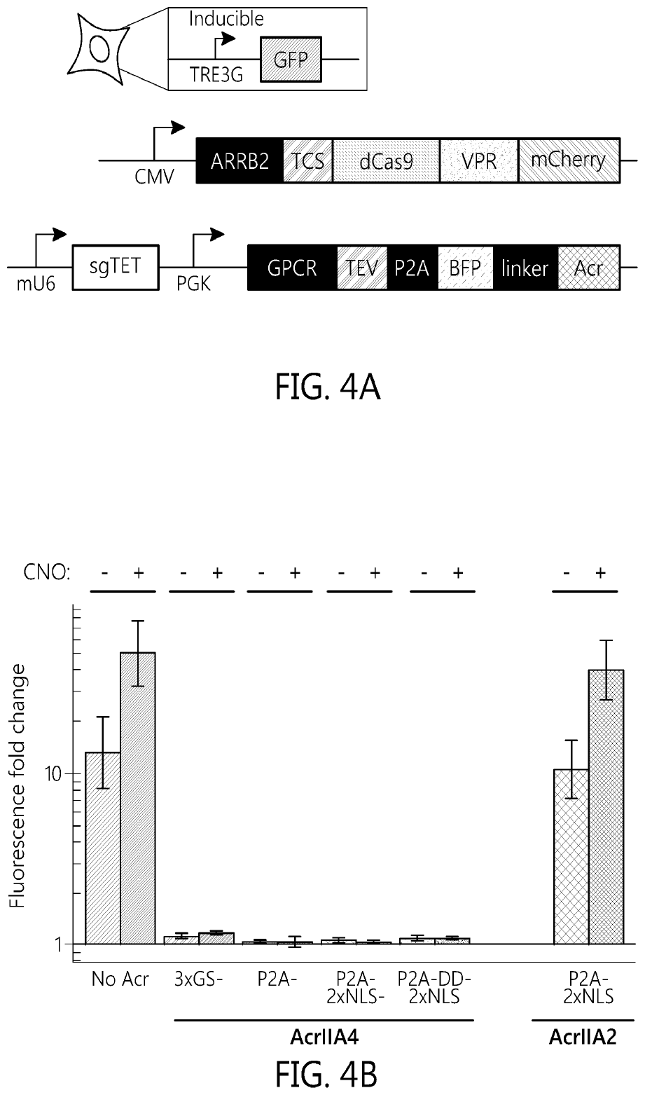

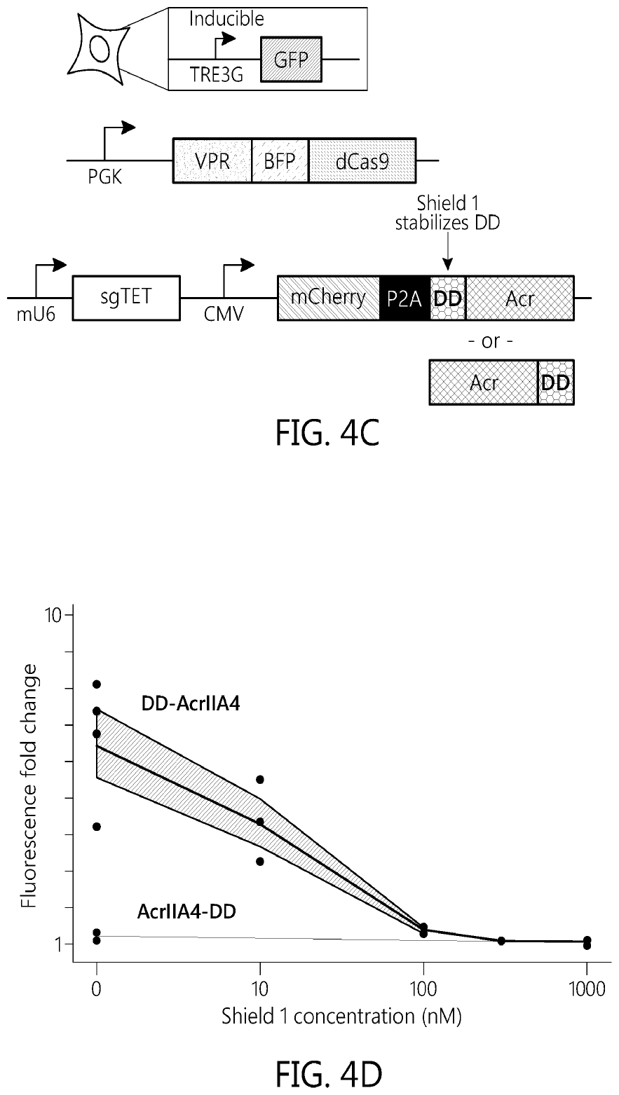

This Example describes experiments performed to examine the use of Acrs in inducible gene regulation systems. It was recently reported that a G-protein-coupled receptor (GPCR)-activated dCas9 gene regulation system (ChaCha system), which combines a GPCR fused to tobacco etch virus protease (TEV) and V2 vasopressin tail and a dCas9-VPR fused to β-arrestin via a TEV cleavage site (TCS). GPCR activation by cognate ligand binding induces recruitment of β-arrestin, which allows for release of dCas9-VPR for nuclear localization and gene activation. AcrIIA4 was incorporated into the ChaCha system via a variety of linkers—3 xGlySer, P2A, nuclear localization signal (NLS) and destabilization domain (DD)—downstream of the GPCR construct (FIG. 4A). While control ChaCha assays demonstrated ligand-inducible gene activation, all of the AcrIIA4 fusions described herein completely inhibited the synthetic device in both on and off states (FIG. 4B).

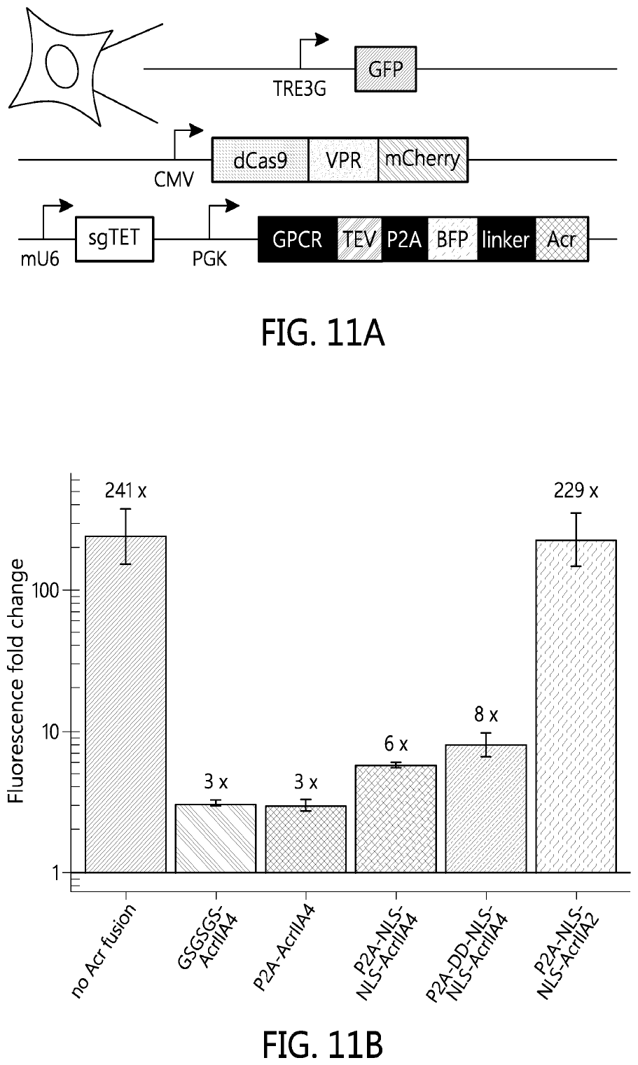

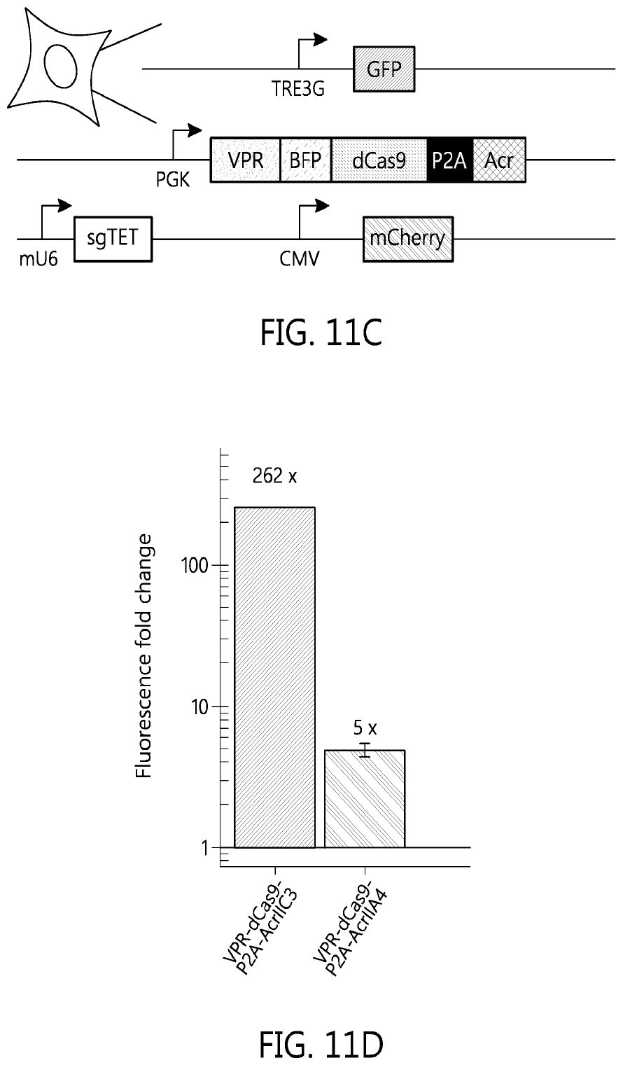

To further understand the nature of this effect, the same set of constructs was tested with a free dCas9-VPR control (FIG. 11A) and again obtained significant reductions in activation (FIG. 11B). By contrast to the Acr ChaCha described herein and previous Acr CRISPRa assays, there remained residual (3- to 8-fold) activation. By comparing the performance of the identical (after cleavage) P2A-AcrIIA4 construct in the assay described herein and in previously described Acr CRISPRa assays, these results can most likely be at least partially explained by the difference in stoichiometric ratios: the CMV (strong) and PGK (weak) promoters are switched (and the GPCR-TEV-P2A-BFP construct is much larger than the mCherry fused to AcrIIA4 in FIGS. 1A-1E). This idea that relative stoichiometry potentially play a role in overall activity was corroborated by an experiment wherein dCas9-VPR and Acr are produced from the same plasmid in roughly a 1:1 ratio (FIG. 11C), resulting in similar amounts of significant, but not total, downregulation of gene expression (FIG. 11D).

Based on these combined results, the performance of various Acr fusions can be assessed: fusing AcrIIA4 to NLSx2 seems to diminish its performance, even relative to a BFP fusion (3 xGlySer linker), and, in accordance with expectations, fusing DD reduces performance further. Similarly, the stronger performance of these construct variants on the ChaCha assay relative to the free dCas9-VPR assay seems to indicate that active free dCas9-VPR molecules in the ChaCha assay (after protease cleavage) are produced in lower amounts relative to constitutive expression of the free dCas9-VPR. Also tested was a P2A-NLSx2-AcrIIA2 construct, which showed no inhibition of CRISPRa even in the ChaCha assays, indicating that this construct is almost completely inactive. The results suggest that switching between experimental or construct configurations might allow for deeper quantitative understanding of both CRISPR-based devices and anti-CRISPR activity.

### Example 7

### Acrs Allows Dosable Control of CRISPR Activity

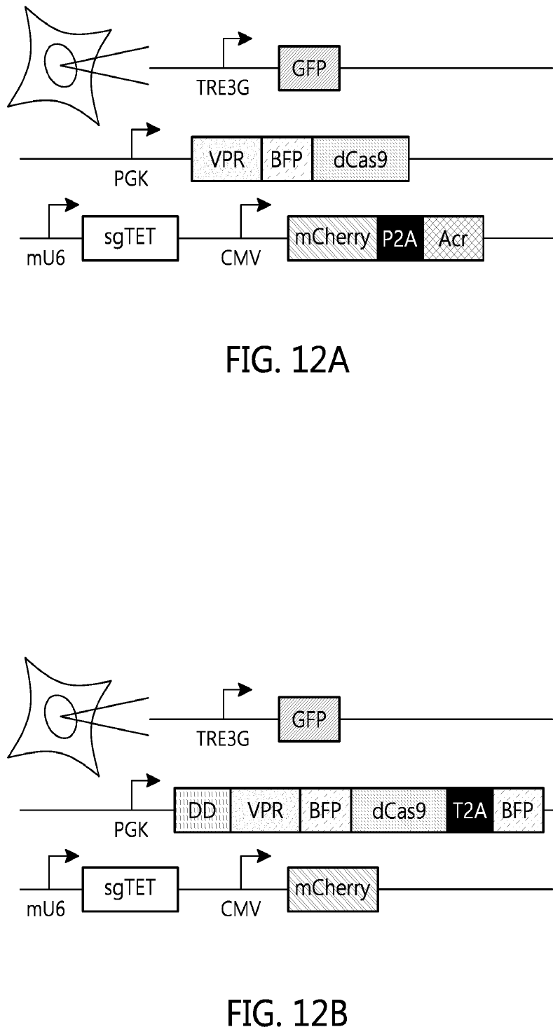

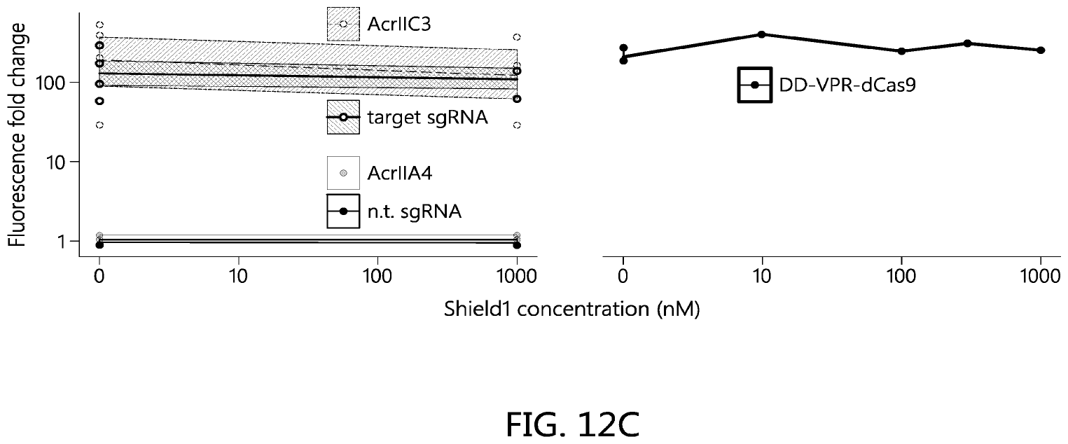

This Example describes experiments performed to further engineer Acrs for inducible control of gene expression. To do this, an engineered inducible destabilization domain (DD) was fused to AcrIIA4 (FIG. 4C). Addition of cognate ligand Shield1 stabilizes DD and the fused AcrIIA4, leading to stronger inhibition of dCas9 activity. The DD-AcrIIA4 constructs were tested using a reporter CRISPRa assay and observed Shield1-dependent switch behavior (FIG. 4d), while normal CRISPRa or Acr lacking DD showed no response (FIGS. 12A and 12C). Fusing DD to the N-terminus of AcrIIA4 showed Shield-1-inducible gene expression, while fusing DD to the C-terminus was ineffective, possibly due to the reduced amount of induced degradation provided by DD at the C-terminus. Also, no change in activation with a DD-VPR-dCas9 construct was observed (FIGS. 12B and 12C). A DD-Cas9 was previously shown to have inducible editing activity; the difference possibly lies in the increased size of the VPR-dCas9 construct and the delivery method (transient transfection versus viral transduction). Notably, another DD variant was found to be ineffective in modulating gene expression when fused to dCas9 in another study, further corroborating that DD provides incomplete control over dCas9-based effector function. These results demonstrate the utility of AcrIIA4 as an easily incorporable and modular tool for engineering inducible dCas9 activity, requiring no re-engineering of the dCas9 construct (and demonstrating superior performance in at least certain contexts).

### Example 8

### Acrs Offers a Means to Genomically “Write-Protect” Cells

This Example describes experiments performed to investigate whether human cells pre-engineered with Acr molecules would become resistant to genome editing, which in turns results in a genome with “write protection” against specific Cas9s. This is because with the broad use of CRISPR for multiple purposes in diverse organisms, including genome editing to correct diseases, generation of genetically modified organisms (GMOs), and field testing of gene drives to eliminate species, it remains a major need to devise new countermeasures that provide prophylactic options to limit genome editing in organisms and protect genome integrity in populations of organisms. In addition, the experimental results described in this Example also provide a proof-of-principle demonstration of the practicality of integrating Acrs in the genome to generate cells that are immune to unlicensed editing applications.

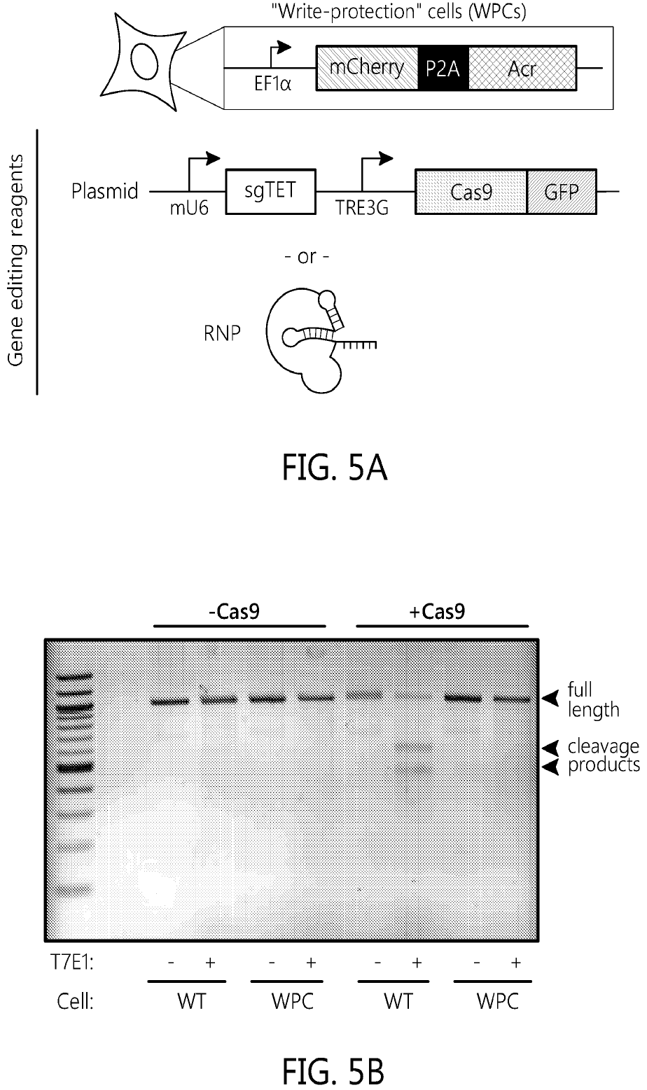

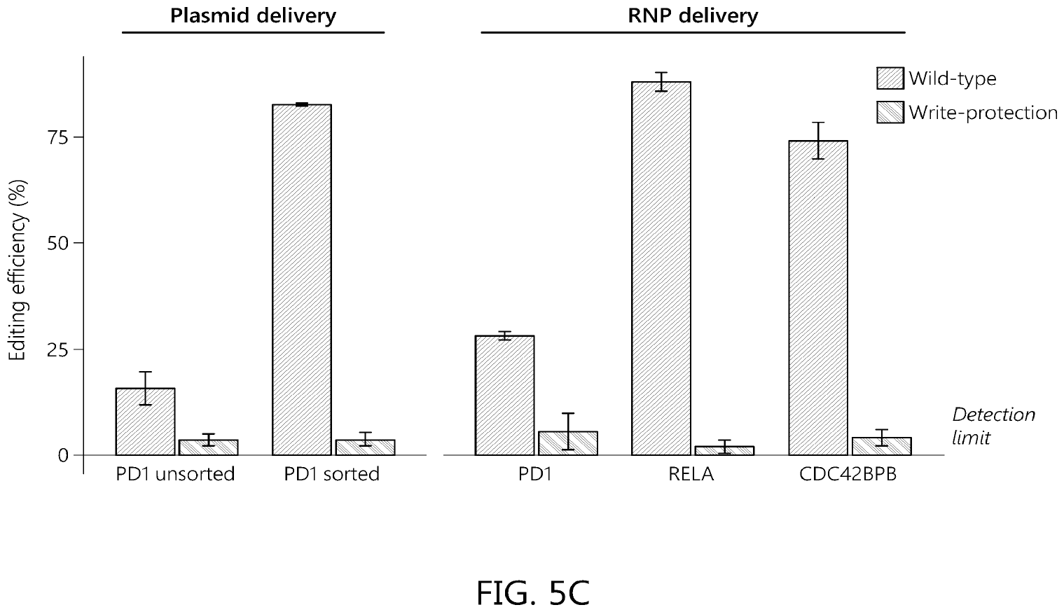

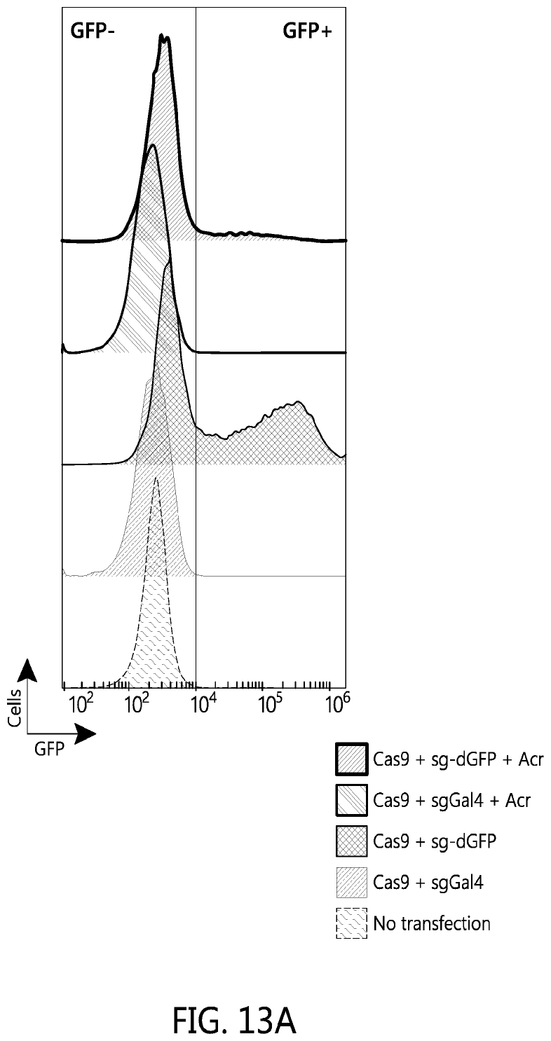

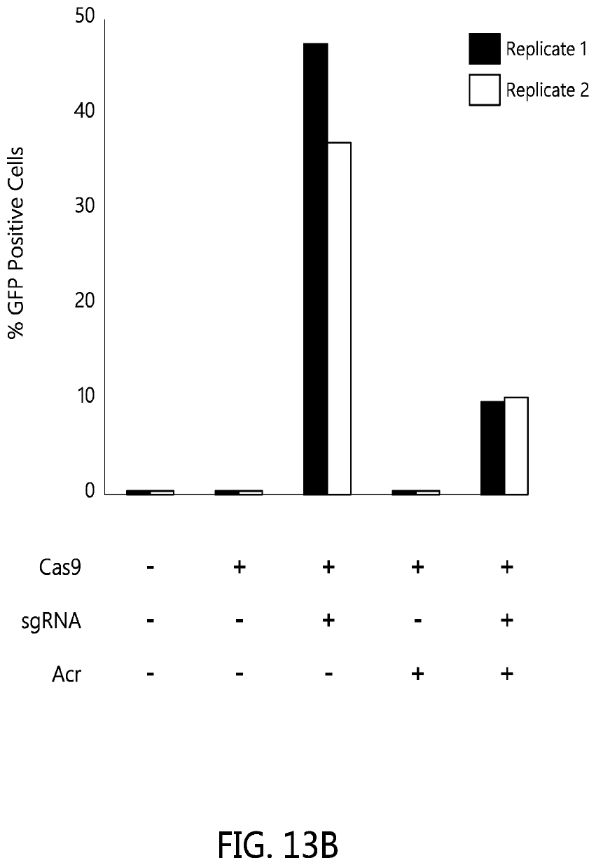

First, the efficacy of AcrIIA4 in a HEK293T reporter system was tested for genome editing and it was noted that co-transfection of AcrIIA4 plasmid resulted in strong, but not total inhibition of genome editing (see, e.g., FIGS. 13A-13B). A lentiviral construct encoding AcrIIA4 was then stably integrated into the genome of HEK293T cells (FIG. 5A), generating write-protected cells (WPCs). Genome editing in WPCs compared to wild-type HEK293T cells was then tested by delivering Cas9 and a sgRNA targeting various genomic loci. Using a plasmid delivery method followed by T7E1 assay, no editing in WPC cells pre-engineered with AcrIIA4 was observed, while wild-type HEK293T cells exhibited strong genome editing (FIG. 5B). Furthermore, various delivery methods using plasmids, with or without subsequent sorting, and ribonucleoprotein complexes (RNPs) were compared with one another, and in all cases genome editing in AcrIIA4-engineered WPC cells was found to be negligible with a similar level below the detection limit of Tracking of Indels by Decomposition (TIDE) analysis (FIG. 5C). These data demonstrate the feasibility of this approach of generating “write protection” in cells to counteract further genome editing, thus laying a foundation to prevent accidental or intentional genome editing in desired cell types.

### Example 9

### Acrs-Based Genetic Circuits Provide Pulsatile Gene Regulation

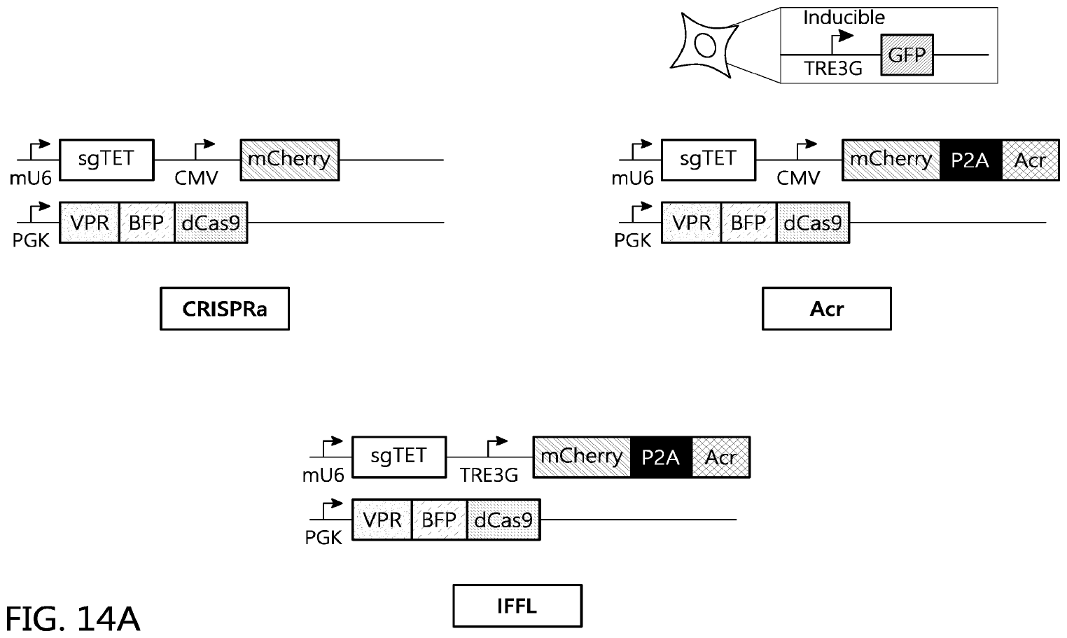

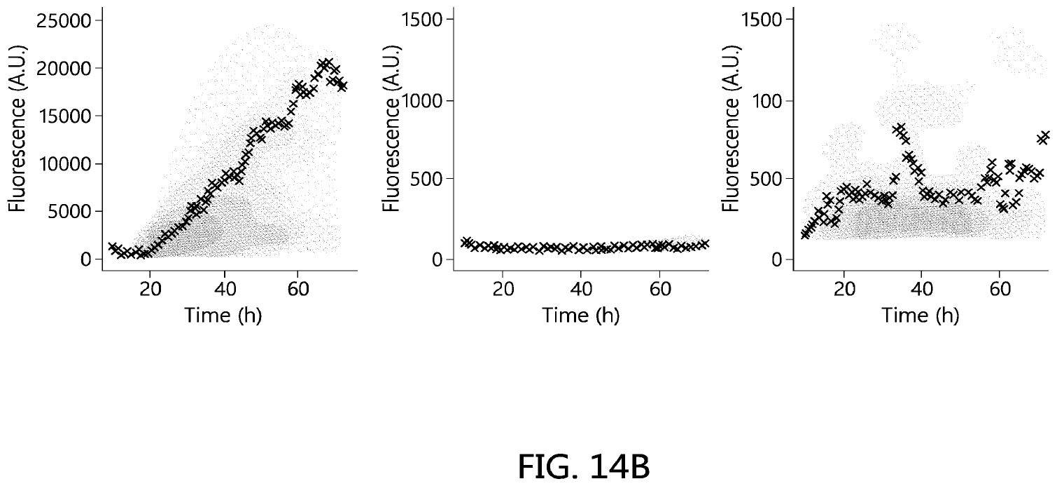

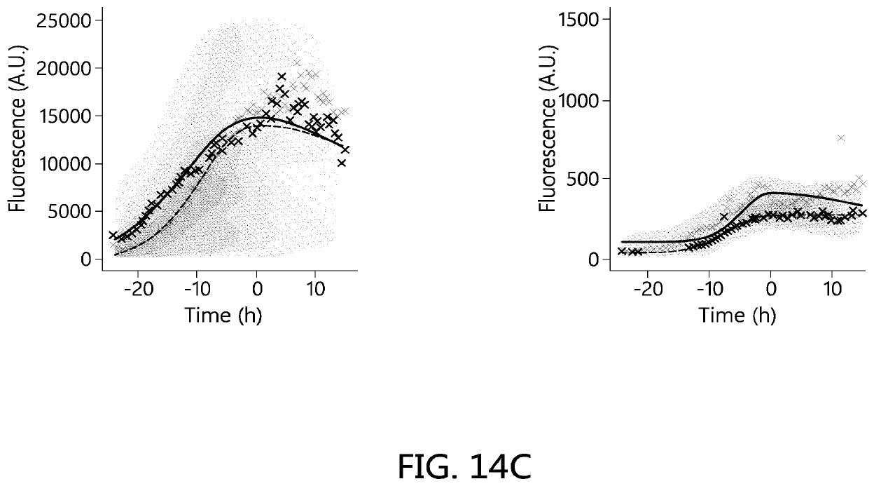

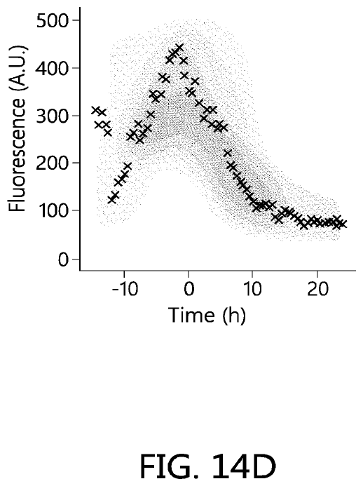

This Example described experiments performed to investigate another potential advantage of Acr-based control of gene regulation, which is that it lends itself as a method to implement pre-programmed genetic circuitry. To test this idea, three simple gene regulation circuits were generated (FIG. 6A): CRISPRa (VPR-dCas9-driven GFP expression), Acr (VPR-dCas9 activity inhibited by constitutive Acr expression), and an incoherent feedforward loop (IFFL) circuit (VPR-dCas9 driving both GFP and Acr expression, resulting in delayed abrogation of CRISPRa activity). To observe the effects of these circuits in a time-dependent manner, a plasmid containing sgRNA and appropriate Acr construct were stably integrated, which triggered the circuit via transient transfection of VPR-dCas9 plasmid (FIG. 14A). The resulting changes in expression of GFP were tracked by live-cell time-lapse microscopy. Though individual cells exhibited heterogeneous responses, clear qualitative differences in phenotypes among the different circuits were evident (see, e.g., Supplementary Movies 1-3).

To more quantitatively understand the behavior of the circuits, an unbiased automated cell tracking image analysis pipeline was integrated, which allowed to follow the GFP trajectory of a cell (or a cluster of cells) over time (see, e.g., FIG. 6B; Supplementary Movie 4). By tracking the GFP fluorescence of these cells, we observed clear CRISPRa and anti-CRISPRa phenotypes in the combined population for the CRISPRa and Acr conditions, while the IFFL condition demonstrated intermediate GFP expression (FIG. 14B).

It was hypothesized that the signal in the bulk population from the IFFL circuit was diluted by variation in the onset time of the circuit, wherein different cells activate at different times (see, e.g., FIG. 6B; Supplementary Movies 3-4), most likely due to biological variation and stochastic differences in delivery time of the circuit-activating VPR-dCas9 plasmid. To account for this variation, each cellular trajectory was fitted to an asymmetric gaussian function, allowing for the alignment of these traces in time relative to the peak point of response. The aligned traces demonstrated a pulse-like phenotype of GFP expression for the IFFL condition (FIG. 6C), whereas a similar alignment procedure revealed sigmoidal behavior with high or low amounts of GFP expression for the CRISPRa and Acr conditions, respectively (FIG. 14C).

Based on fits to the IFFL condition, descriptions of average circuit response were estimated, including pulse width (half-rise time ˜6 h, half-decay time ˜10 h) and amplitude (peak expression ˜5-fold relative to basal level). Also tested was the IFFL condition without stably integrating the sgRNA plasmid and observed similar pulse behavior in a percentage of cells, indicating the possibility of achieving programmed circuit behavior solely via transient delivery of DNA. The ability to implement “pulse generator” circuits using Acrs highlights their utility in synthetic circuit engineering, which can provide a powerful method to control genes in a highly programmable and dynamic manner.

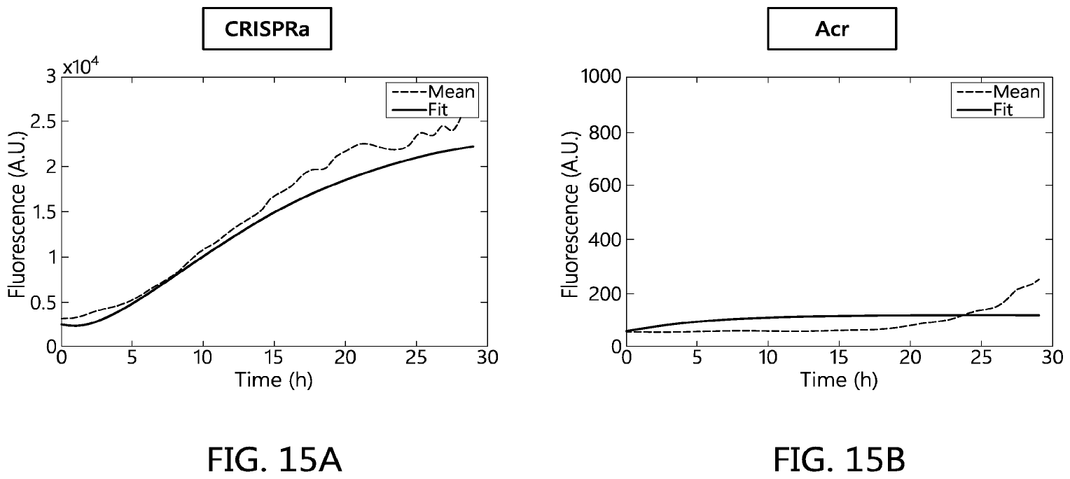

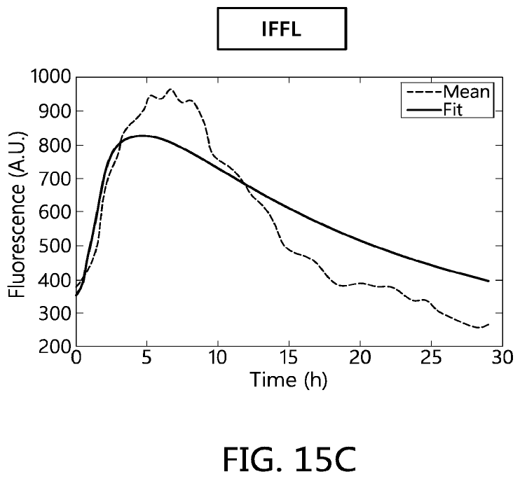

As described in greater detail below, a simple computational model was built in order to further understand the basis of the IFFL circuit behavior see, e.g., Examples 2 and 9). The model was parameterized via sequential fits to experimental data, which allowed to recapitulate the qualitative behavior of all three implemented circuits (FIGS. 15A-15C). Theoretical predictions of the effects of perturbing various parameters on the performance of the circuit were subsequently generated (FIGS. 6D-6J). Notably, parameters such as VPR-dCas9 and Acr production rate, as well as Acr strength of interaction, were predicted as major factors that control amplitude of the generated pulse. These results provide semi-quantitative predictions of how circuit behavior can be rationally altered and lay the groundwork for further implementation of genetic circuits with tunable dynamics and responses.

### Example 10

### Inducible Degradation of a DD-Acr Polypeptide (ArcIJA4) Fusion Using Shield1

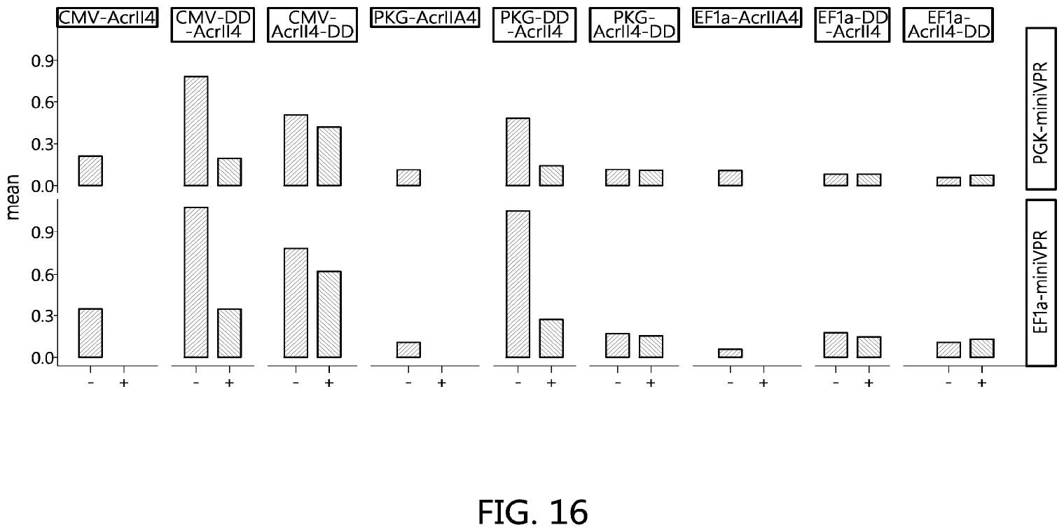

FIG. 16 schematically summarizes the results of experiments performed to demonstrate the degradation of a DD-Acr polypeptide (AcrIIA4) fusion in the presence of Shield1. To further characterize the activity of degradation-based control, a systematic comparison of two versions of inducible CRISPRa activity for N-terminal and C-terminal fusions of DD to AcrIIA4 across varying expression levels of Acr and dCas9. The experiments were performed with transient transfection of plasmids bearing the fusions expressed from various promoters (CMV-, PGK-, EF1a-). It was observed that the modulation strength was dependent on the relative expression strengths of the relevant molecules: higher modulations were observed for lower levels expression of Acr and higher levels of expression of dCas9. It was further confirmed that the C-terminal fusion of DD to Acr demonstrated lower modulation levels than the N-terminal fusion across expression levels (see, e.g., FIG. 16).

Also compared was the ability of DD to modulate CRISPRa activity when fused to dCas9-miniVPR. As shown in FIG. 17, it was confirmed that the N-terminal DD-dCas9-miniVPR fusion showed low-to-negligible levels of modulation. The C-terminal dCas9-miniVPR-DD demonstrated Shield1-mediated increase in CRISPRa activity, but also high levels of activity in the absence of Shield1. These experimental results demonstrate that degron-mediated control of Acr allows for access to different regimes of CRISPR activity, including lower leakiness.

### Example 11

### Genome Editing Efficiencies at Two Different Loci

FIG. 18 schematically summarizes the results of experiments performed to demonstrate genome editing efficiencies at two different loci. In these experiments, a lentiviral construct encoding AcrIIA4 was stably integrated into the genome of human induced pluripotent stem cells (iPSCs), generating write-protected cells (WPCs). Genome editing in WPCs compared to wild-type iPSCs was then tested by delivering Cas9 and a sgRNA targeting various genomic loci via RNPs (see, e.g., FIG. 18). In all cases, genome editing in AcrIIA4-engineered WPC cells was found to be negligible with a similar level below the detection limit by TIDE analysis. These data further demonstrate the applicability of this approach of generating “write protection” in cells to counteract further genome editing, thus laying a foundation to prevent accidental or intentional genome editing in desired cell types.

### Example 12

### HK-AcrVA and Gate

FIG. 19 schematically summarizes the results of experiments performed to demonstrate HK-AcrVA and gate. In these experiments, a dCas12a-miniVPR protein with a leucine zipper was split into two portions: N-terminal portion (A) and C-terminal portion (B) that could spontaneously recombine into an active dCas12a-miniVPR protein when both portions are expressed. dCas12a-miniVPR activity is inhibited in the presence of a third polypeptide, i.e., AcrVA1 protein (C). Therefore, the three proteins constitute a logic gate, where both portions of dCas12a-miniVPR protein (A and B) must be expressed and AcrVA1 is absent in order to obtain output of the device (A AND B AND NOT C). Reporter cells were transfected with various combinations of the plasmids and gene activation levels assessed. Significant levels of gene expression were obtained only in the presence of both portions of dCas12a-miniVPR and in the absence of AcrVA1.

Supplementary movies: Representative movies of live-cell genetic circuit experiments. Timestamps correspond to hours post-transfection.

Live-cell genetic circuit experiments were tested in CRISPRa condition (unperturbed GFP activation). The results of these experiments were further documented in a video (Supplementary Movie 1) which depicts cells transfected with CRISPRa components and no Acr continuously increased in GFP expression over time, reaching a high level of steady-state expression

Live-cell genetic circuit experiments were also tested in Acr condition (constitutive expression of Acr inhibits GFP activation). The results of these experiments were further documented in a video (Supplementary Movie 2), which depicts cells with constitutively expressed Acr transfected with CRISPRa components along failed to express significant GFP at all time points.

Live-cell genetic circuit experiments were also tested in IFFL condition (induced activation of GFP and Acr). The results of these experiments were further documented in a video (Supplementary Movie 3), which depicts cells with inducible Acr transfected with CRISPRa components demonstrate an increase followed by a decrease in GFP expression. In this video, selected cells demonstrating pulsatile reporter expression are manually annotated.

Computationally selected cell-tracking traces of IFFL condition was generated from image analysis pipeline. The results of these experiments were further documented in a video (Supplementary Movie 4), which demonstrates that a computational analysis pipeline allows for tracking of single cell fluorescence over time. This movie depicts a collection of single-cell movies that demonstrate the pulsatile gene expression phenotype.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### REFERENCES

1. Dominguez, A. A., Lim, W. A. & Qi, L. S. Beyond editing: repurposing CRISPR-Cas9 for precision genome regulation and interrogation. Nat. Rev. Mol. Cell Biol. 17, 5-15 (2016).
2. Komor, A. C., Badran, A. H. & Liu, D. R. CRISPR-Based Technologies for the Manipulation of Eukaryotic Genomes. Cell 168, 20-36 (2017).
3. Nandagopal, N. & Elowitz, M. B. Synthetic biology: integrated gene circuits. Science 333, 1244-8 (2011).
4. Sedlmayer, F., Aubel, D. & Fussenegger, M. Synthetic gene circuits for the detection, elimination and prevention of disease. Nat. Biomed. Eng. 2, 399-415 (2018).
5. Nielsen, A. A. & Voigt, C. A. Multi-input CRISPR/Cas genetic circuits that interface host regulatory networks. Mol. Syst. Biol. 10, 763-763 (2014).
6. Gander, M. W., Vrana, J. D., Voje, W. E., Carothers, J. M. & Klavins, E. Digital logic circuits in yeast with CRISPR-dCas9 NOR gates. Nat. Commun. 8, 15459 (2017).
7. Gao, Y. et al. Complex transcriptional modulation with orthogonal and inducible dCas9 regulators. Nat. Methods 13, 1043-1049 (2016).
8. Kipniss, N. H. et al. Engineering cell sensing and responses using a GPCR-coupled CRISPR-Cas system. Nat. Commun. 8, 2212 (2017).
9. Baeumler, T. A., Ahmed, A. A. & Fulga, T. A. Engineering Synthetic Signaling Pathways with Programmable dCas9-Based Chimeric Receptors. Cell Rep. 20, 2639-2653 (2017).
10. Liu, Y. et al. Engineering cell signaling using tunable CRISPR-Cpf1-based transcription factors. Nat. Commun. 8, 2095 (2017).
11. Chen, T. et al. Chemically Controlled Epigenome Editing through an Inducible dCas9 System. J. Am. Chem. Soc. 139, 11337-11340 (2017).
12. Kleinjan, D. A., Wardrope, C., Nga Sou, S. & Rosser, S. J. Drug-tunable multidimensional synthetic gene control using inducible degron-tagged dCas9 effectors. Nat. Commun. 8, 1191 (2017).
13. Maxwell, K. L. The Anti-CRISPR Story: A Battle for Survival. Mol. Cell 68, 8-14 (2017).
14. Pawluk, A. et al. Naturally Occurring Off-Switches for CRISPR-Cas9. Cell 167, 1829-1838.e9 (2016).
15. Rauch, B. J. et al. Inhibition of CRISPR-Cas9 with Bacteriophage Proteins. Cell 168, 150-158.e10 (2017).
16. Dong, D. et al. Structural basis of CRISPR-SpyCas9 inhibition by an anti-CRISPR protein. Nature 546, 436-439 (2017).
17. Shin, J. et al. Disabling Cas9 by an anti-CRISPR DNA mimic. Sci. Adv. 3, e1701620 (2017).
18. Harrington, L. B. et al. A Broad-Spectrum Inhibitor of CRISPR-Cas9. Cell 170, 1224-1233.e15 (2017).
19. Marshall, R. et al. Rapid and Scalable Characterization of CRISPR Technologies Using an E. coli Cell-Free Transcription-Translation System. Mol. Cell 69, 146-157.e3 (2018).
20. Bubeck, F. et al. Engineered anti-CRISPR proteins for optogenetic control of CRISPR-Cas9. Nat. Methods 15, 924-927 (2018).
21. Liu, X. S. et al. Rescue of Fragile X Syndrome Neurons by DNA Methylation Editing of the FMR1 Gene. Cell 172, 979-992.e6 (2018).
22. Qi, L. S. et al. Repurposing CRISPR as an RNA-Guided Platform for Sequence-Specific Control of Gene Expression. Cell 152, 1173-1183 (2013).
23. Peters, J. M. et al. A Comprehensive, CRISPR-based Functional Analysis of Essential Genes in Bacteria. Cell 165, 1493-1506 (2016).
24. Rock, J. M. et al. Programmable transcriptional repression in mycobacteria using an orthogonal CRISPR interference platform. Nat. Microbiol. 2, 16274 (2017).
25. Gilbert, L. A. et al. CRISPR-mediated modular RNA-guided regulation of transcription in eukaryotes. Cell 154, 442-51 (2013).
26. Zalatan, J. G. G. et al. Engineering Complex Synthetic Transcriptional Programs with CRISPR RNA Scaffolds. Cell 160, 339-350 (2014).
27. Chavez, A. et al. Highly efficient Cas9-mediated transcriptional programming. Nat. Methods 12, 326-328 (2015).
28. Hynes, A. P. et al. An anti-CRISPR from a virulent streptococcal phage inhibits Streptococcus pyogenes Cas9. Nat. Microbiol. 2, 1374-1380 (2017).
29. Hynes, A. P. et al. Widespread anti-CRISPR proteins in virulent bacteriophages inhibit a range of Cas9 proteins. Nat. Commun. 9, 2919 (2018).
30. Li, J., Xu, Z., Chupalov, A. & Marchisio, M. A. Anti-CRISPR-based biosensors in the yeast S. cerevisiae. J. Biol. Eng. 12, 11 (2018).
31. Liu, Z. et al. Systematic comparison of 2A peptides for cloning multi-genes in a polycistronic vector. Sci. Rep. 7, 2193 (2017).
32. Basgall, E. M. et al. Gene drive inhibition by the anti-CRISPR proteins AcrIIA2 and AcrIIA4 in Saccharomyces cerevisiae. Microbiology 164, 464-474 (2018).
33. Banaszynski, L. A., Chen, L., Maynard-Smith, L. A., Ooi, A. G. L. & Wandless, T. J. A Rapid, Reversible, and Tunable Method to Regulate Protein Function in Living Cells Using Synthetic Small Molecules. Cell 126, 995-1004 (2006).
34. Chu, B. W., Banaszynski, L. A., Chen, L. & Wandless, T. J. Recent progress with FKBP-derived destabilizing domains. Bioorg. Med. Chem. Lett. 18, 5941-5944 (2008).
35. Senturk, S. et al. Rapid and tunable method to temporally control genome editing based on conditional Cas9 stabilization. Nat. Commun. 8, 14370 (2017).
36. Brinkman, E. K., Chen, T., Amendola, M. & van Steensel, B. Easy quantitative assessment of genome editing by sequence trace decomposition. Nucleic Acids Res. 42, e168-e168 (2014).
37. Doron, S. et al. Systematic discovery of antiphage defense systems in the microbial pangenome. Science (80-.). 359, eaar4120 (2018).
38. Watters, K. E., Fellmann, C., Bai, H. B., Ren, S. M. & Doudna, J. A. Systematic discovery of natural CRISPR-Cas12a inhibitors. Science (80-.). 362, 236-239 (2018).
39. Marino, N. D. et al. Discovery of widespread type I and type V CRISPR-Cas inhibitors. Science (80-.). 362, 240-242 (2018).
40. Galanie, S., Thodey, K., Trenchard, I. J., Filsinger Interrante, M. & Smolke, C. D. Complete biosynthesis of opioids in yeast. Science 349, 1095-100 (2015).
41. Gantz, V. M. et al. Highly efficient Cas9-mediated gene drive for population modification of the malaria vector mosquito Anopheles stephensi. Proc. Natl. Acad. Sci. 112, 201521077 (2015).
42. Hammond, A. et al. A CRISPR-Cas9 gene drive system targeting female reproduction in the malaria mosquito vector Anopheles gambiae. Nat. Biotechnol. 34, 78-83 (2016).
43. Basu, S., Mehreja, R., Thiberge, S., Chen, M.-T. & Weiss, R. Spatiotemporal control of gene expression with pulse-generating networks. Proc. Natl. Acad. Sci. U.S.A 101, 6355-60 (2004).
44. Mandegar, M. A. et al. CRISPR Interference Efficiently Induces Specific and Reversible Gene Silencing in Human iPSCs. Cell Stem Cell 18, 541-553 (2016).
45. Entian, K.-D. & Kötter, P. in Methods in Microbiology 36, 629-666 (Academic Press, 2007).
46. Hendel, A. et al. Chemically modified guide RNAs enhance CRISPR-Cas genome editing in human primary cells. Nat. Biotechnol. 33, 985-989 (2015).
47. Fonfara I., Le Rhun A., Chylinski K., Makarova K. S., Lécrivain A. L., Bzdrenga J., Koonin E. V., Charpentier E., Phylogeny of Cas9 determines functional exchangeability of dual-RNA and Cas9 among orthologous type II CRISPR-Cas systems. Nucleic Acids Res. 42(4): 2577-2590, (2014).
48. Morsut L, Roybal K T, Xiong X, Gordley R M, Coyle S M, Thomson M, and Lim W A. Engineering Customized Cell Sensing and Response Behaviors Using Synthetic Notch Receptors. Cell. 2016 Feb. 11; 164(4): 780-791.
49. Roybal K T, Jasper Z. Williams, Leonardo Morsut, Levi J. Rupp, Isabel Kolinko, Joseph H. Choe, Whitney J. Walker, Krista A. McNally, and Wendell A. Lim. Engineering T cells with Customized Therapeutic Response Programs Using Synthetic Notch Receptors. Cell. 2016 Oct. 6; 167(2): 419-432.
50. Koonin E. V., Makarova K. S., and Zhang F. Diversity, classification and evolution of CRISPR-Cas systems. Current Opinion in Microbiology, Vol. 37, June 2017, pp. 67-78.
51. Zhang F., Song G., and Tian Y. Anti-CRISPRs: The natural inhibitors for CRISPR-Cas systems. Animal Model Exp Med. 2019 June; 2(2): 69-75.

What is claimed is:

1. An engineered cell comprising:
(a) one or more components of a CRISPR system comprising (i) a CRISPR-associated (Cas) endonuclease or a first nucleic acid encoding the Cas endonuclease, and (ii) at least one guide RNA (gRNA); and
(b) an anti-CRISPR (Acr) polypeptide or a second nucleic acid encoding the Acr polypeptide,
wherein the Acr polypeptide modulates an activity of the CRISPR system in an inducible and/or programmable manner.

2. The engineered cell of claim 1, wherein the at least one gRNA is encoded by a sequence incorporated in the first nucleic acid or in the second nucleic acid.

3. The engineered cell of any one of claims 1 to 2, wherein the CRISPR system further comprising a donor template that comprises a sequence encoding a gene-of-interest (GOI).

4. The engineered cell of any one of claims 1 to 3, wherein the CRISPR system comprises a Class 2 Cas endonuclease or a derivative thereof.

5. The engineered cell of claim 4, wherein the Class 2 Cas endonuclease is a Cas9 endonuclease selected from the group consisting of a Streptococcus pyogenes Cas9, a Streptococcus thermophiles Cas9, a Staphylococcus aureus Cas9, a Brackiella oedipodis Cas9, a Neisseria meningitidis Cas9, a Haemophilus influenzae Cas9, a Simonsiella muelleri Cas9, a Ralstonia solanacearum Cas9, a Francisella novicida Cas9, and a Listeria monocytogenes Cas9, or a derivative of any thereof.

6. The engineered cell of any one of claims 4 to 5, wherein the Cas9 derivative is a nuclease-deficient Cas9 polypeptide (dCas9).

7. The engineered cell of any one of claims 1 to 3, wherein the CRISPR system comprises a Type V Cas endonuclease or a derivative thereof.

8. The engineered cell of claim 7, wherein the Type V Cas endonuclease is a Cas12 endonuclease selected from the group consisting of a Francisella novicida Cas12, a Lachnospiraceae bacterium ND2006 Cas12, and an Acidaminococcus sp. BV3L6 Cas12a, or a derivative of any thereof.

9. The engineered cell of claim 8, wherein the Cas12 derivative is a nuclease-deficient Cas12 polypeptide (dCas12).

10. The engineered cell of any one of claims 1 to 6, wherein the Cas endonuclease is operably linked to an effector domain is selected from the group consisting of an activation domain, a repression domain, a protein modification domain, a histone modification domain, a DNA modification domain, a RNA modification domain, and a heterodimerization domain.

11. The engineered cell of any one of claims 1 to 10, wherein the CRISPR system is a CRISPR-based gene regulation system.

12. The engineered cell of claim 11, wherein the CRISPR-based gene regulation system is a CRISPR-based gene activation (CRISPRa) system.

13. The engineered cell of claim 12, wherein the CRISPRa system comprises a dCas9 polypeptide or a dCas12 polypeptide operably linked to a transcriptional activation domain.

14. The engineered cell of claim 13, wherein the transcriptional activation domain is selected from the group consisting of a VP16 activation domain, a VP64 activation domain, a p65 activation domain, a MyoD1 activation domain, a HSF1 activation domain, a RTA activation domain, a SETT/9 activation domain, a VP64-p65-Rta (VPR) activation domain, a mini VPR activation domain, a yeast GAL4 activation domain, a yeast HAP1 activation domain, and a histone acetyltransferase.

15. The engineered cell of claim 11, wherein the CRISPR-based gene regulation system is a CRISPR-based gene interference (CRISPRi) system.

16. The engineered cell of claim 15, wherein the CRISPRi system comprises a dCas9 polypeptide or a dCas12 polypeptide operably linked to a repression domain.

17. The engineered cell of claim 16, wherein the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3A repressor domain, and a DNMT3L repressor domain.

18. The engineered cell of any one of claims 1 to 10, wherein the CRISPR system is a CRISPR-based genome editing system.

19. The engineered cell of claim 18, wherein the activity of the CRISPR-based genome editing system is selected from the group consisting of target polynucleotide binding, target polynucleotide double-strand break creation, target polynucleotide nicking (single-strand DNA cleavage), and target polynucleotide modification.

20. The engineered cell of claim 19, wherein the target polynucleotide modification is selected from the group consisting of insertion of at least one nucleotide, deletion of at least one nucleotide, substitution of at least one nucleotide, and chemical alteration of at least one nucleotide.

21. The engineered cell of any one of claims 1 to 20, wherein the Acr polypeptide comprises a bacteriophage-derived Acr polypeptide.

22. The engineered cell of claim 21, wherein the bacteriophage-derived Acr polypeptide is selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof.

23. The engineered cell of claim 22, wherein the functional variants have at least 80% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, and AcrVA5.

24. The engineered cell of any one of claims 1 to 23, wherein the expression and/or activity of the Acr polypeptide and/or the CRISPR system is inducible or programmable.

25. The engineered cell of any one of claims 1 to 24, wherein the Acr polypeptide and/or the Cas endonuclease is operably linked to an inducible destabilization domains (DD) selected from the group consisting of a DD from the rapamycin-binding protein (FKBP12), a DD from dihydrofolate reductase (DHFR), a DD from estrogen receptor ligand binding domain (ERLBD), and a DD from estrogen receptor (ER50).

26. The engineered cell of claim 25, wherein the DD is from FKBP12 and the stabilization of the DD is induced by addition of rapamycin analogue compound Shield1.

27. The engineered cell of any one of claims 1 to 26, wherein the Acr polypeptide and/or the Cas endonuclease is operably linked to a proteolytic cleavage site.

28. The engineered cell of claim 27, wherein the proteolytic cleavage site can be cleaved by a protease selected from the group consisting of a tobacco etch virus (TEV) protease, a porcine teschovirus-1 2A (P2A) protease, a foot-and-mouth disease virus (FMDV) 2A (F2A) protease, an Equine Rhinitis A Virus (ERAV) 2A (E2A) protease, a Thosea asigna virus 2A (T2A) protease, a cytoplasmic polyhedrosis virus 2a (BmCPV2A) protease, a Flacherie Virus 2A (BmIFV2A) protease, thrombin, PreScission™ protease, a glutamyl endopeptidase, an Epstein-Barr virus protease, a matrix metalloproteinase 2 (MMP-2), a matrix metalloproteinase 1 (MMP-1), a membrane type 1 matrixmetalloproteinase (MT-MMP), a stromelysin 3 (or MMP-11), a matrix metalloproteinase 13 (collagenase-3), an MMP-3 (stromelysin), an MMP-7 (matrilysin), MMP-9, an NS3 protease, and a thermolysin-like MMP.

29. The engineered cell of any one of claims 1 to 28, wherein the Acr polypeptide and/or the Cas endonuclease is further linked to one or more of the following: a nuclear localization signal (NLS), a G-protein-coupled receptor (GPCR), a Gly-Ser linker, and a synthetic Notch receptor.

30. The engineered cell of any one of claims 1 to 29, wherein the Acr polypeptide and/or the one or more components of the CRISPR system are expressed episomally in the cell.

31. The engineered cell of any one of claims 1 to 29, wherein the Acr polypeptide and/or the one or more components of the CRISPR system are expressed from nucleic acids stably integrated in the genome of the cell.

32. The engineered cell of any one of claims 30 to 31, wherein the expression of the Acr polypeptide and/or the one or more components of the CRISPR system is independently under control of a constitutive promoter, a repressible promoter, or an inducible promoter.

33. The engineered cell of claim 32, wherein the constitutive promoter is selected from the group consisting of a SV40 promoter, a CMV promoter, a PGK promoter, a ubiquitin C (UBC) promoter, an EF1A promoter, and a CAGG promoter, and a SFFV promoter.

34. The engineered cell of claim 32, wherein the inducible promoter is selected from the group consisting of a TRE3G inducible promoter, a tetracycline-regulated promoter, a steroid-regulated promoter, a metal-regulated promoter, an estrogen receptor-regulated promoter, and a UAS inducible promoter.

35. The engineered cell of any one of claims 32 to 34, wherein the expression of the Acr polypeptide and/or the one or more components of a CRISPR system is under control of the same promoter.

36. The engineered cell of any one of claims 1 to 35, comprising:
(a) a first nucleic acid molecule encoding (i) an Acr polypeptide and (ii) a single guide RNA (sgRNA); and
(b) a second nucleic acid molecule encoding a nuclease-deficient Cas polypeptide (dCas) fused to an effector domain;
wherein the Acr polypeptide inhibits the activity of the dCas-effector domain fusion and wherein the expression of the Acr polypeptide and the CRISPRa system is under control of the same promoter.

37. The engineered cell of claim 35, wherein the first nucleic acid molecule is stably integrated into the genome of the cell.

38. The engineered cell of claim 35, wherein the first nucleic acid molecule is maintained episomally in the cell.

39. The engineered cell of any one of claims 1 to 38, wherein the cell is selected from the group consisting of a microbial cell, a fungal cell, a plant cell, and an animal cell, mammalian cell.

40. The engineered cell of claim 39, wherein the animal cell is a mammalian cell.

41. The engineered cell of claim 40, wherein the mammalian cell is a human cell.

42. The engineered cell of claim 41, wherein the human cell is a stem cell.

43. The engineered cell of claim 42, wherein the stem cell is a human-induced pluripotent stem cell (hiPSC).

44. A kit comprising:
(a) a Cas endonuclease of a CRISPR system or a first nucleic acid encoding the Cas endonuclease; and
(b) an anti-CRISPR (Acr) polypeptide or a second nucleic acid encoding the Acr polypeptide,
wherein the Acr polypeptide modulates an activity of the CRISPR system in an inducible and/or programmable manner.

45. The kit of claim 44, further comprising a sequence encoding at least one gRNA and/or a sequence encoding a donor template encoding a gene-of-interest (GOI).

46. The kit of claim 45, wherein the sequence encoding the sgRNA and/or the sequence encoding a donor template is incorporated in the first nucleic acid.

47. The kit of any one of claims 45 to 46, wherein the sequence encoding the gRNA and/or the sequence encoding a donor template is incorporated in the second nucleic acid.

48. A method for selectively modulating an activity of a CRISPR system in a cell, the method comprising providing to the cell:
(a) one or more components of a CRISPR system comprising (i) a Cas endonuclease or a first nucleic acid encoding the Cas endonuclease, and (ii) at least one gRNA; and
(b) an anti-CRISPR (Acr) polypeptide or a second nucleic acid encoding the Acr polypeptide,
wherein the Acr polypeptide modulates an activity of the CRISPR system in an inducible and/or programmable manner.

49. The method of claim 48, wherein the at least one gRNA is encoded by a sequence incorporated in the first nucleic acid or in the second nucleic acid.

50. The method of any one of claims 48 to 49, wherein the CRISPR system further comprising a donor template that comprises a sequence encoding a gene-of-interest (GOI).

51. The method of any one of claims 48 to 50, wherein the CRISPR system comprises a Class 2 Cas endonuclease or a derivative thereof.

52. The method of any one of claims 48 to 51, wherein the CRISPR system comprises a Type II Cas endonuclease or a derivative thereof.

53. The method of claim 52, wherein the Type II Cas endonuclease is a Cas9 endonuclease selected from the group consisting of a Streptococcus pyogenes Cas9, a Streptococcus thermophiles Cas9, a Staphylococcus aureus Cas9, a Brackiella oedipodis Cas9, a Neisseria meningitidis Cas9, a Haemophilus influenzae Cas9, a Simonsiella muelleri Cas9, a Ralstonia solanacearum Cas9, a Francisella novicida Cas9, and a Listeria monocytogenes Cas9, or a derivative of any thereof.

54. The method of any one of claims 52 to 53, wherein the Cas9 derivative is a nuclease-deficient Cas9 polypeptide (dCas9).

55. The method of any one of claims 48 to 51, wherein the CRISPR system comprises a Type V Cas endonuclease or a derivative thereof.

56. The engineered cell of claim 55, wherein the Type V Cas endonuclease is a Cas12 endonuclease selected from the group consisting of a Francisella novicida Cas12, a Lachnospiraceae bacterium ND2006 Cas12, and an Acidaminococcus sp. BV3L6 Cas12a, or a derivative thereof.

57. The engineered cell of any one of claims 55 to 56, wherein the Cas12 derivative is a nuclease-deficient Cas12 polypeptide (dCas12).

58. The method of any one of claims 48 to 57, wherein the Cas endonuclease is operably linked to an effector domain selected from the group consisting of an activation domain, a repression domain, a protein modification domain, a histone modification domain, a DNA modification domain, a RNA modification domain, and a heterodimerization domain.

59. The method of any one of claims 48 to 58, wherein the CRISPR system is a CRISPR-based gene regulation system.

60. The method of claim 59, wherein the CRISPR-based gene regulation system is a CRISPR-based gene activation (CRISPRa) system.

61. The method of claim 60, wherein the CRISPRa system comprises a dCas9 or a dCas12 polypeptide operably linked to a transcriptional activation domain.

62. The method of claim 61, wherein the transcriptional activation domain is selected from the group consisting of a VP16 activation domain, a VP64 activation domain, a p65 activation domain, a MyoD1 activation domain, a HSF1 activation domain, a RTA activation domain, a SETT/9 activation domain, a VP64-p65-Rta (VPR) activation domain, a mini VPR domain, a yeast GAL4 activation domain, a yeast HAP1 activation domain, and a histone acetyltransferase.

63. The method of any one of claims 48 to 58, wherein the CRISPR-based gene regulation system is a CRISPR-based gene interference (CRISPRi) system.

64. The method of claim 63, wherein the CRISPRi system comprises a dCas9 polypeptide or a dCas12 polypeptide operably linked to a repression domain.

65. The method of claim 64, wherein the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3A repressor domain, and a DNMT3L repressor domain.

66. The method of any one of claims 48 to 58, wherein the CRISPR-based gene regulation system is a CRISPR-based genome editing system.

67. The method of claim 66, wherein the activity of the CRISPR-based genome editing system is selected from the group consisting of target polynucleotide binding, target polynucleotide double-strand break creation, target polynucleotide nicking (single-strand DNA cleavage), and target polynucleotide modification.

68. The method of claim 67, wherein the target polynucleotide modification is selected from the group consisting of insertion of at least one nucleotide, deletion of at least one nucleotide, substitution of at least one nucleotide, and chemical alteration of at least one nucleotide.

69. The method of any one of claims 48 to 68, wherein the Acr polypeptide comprises a bacteriophage-derived Acr polypeptide.

70. The method of claim 69, wherein the bacteriophage-derived Acr polypeptide is selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, AcrVA5, and functional variants of any thereof.

71. The method of claim 70, wherein the functional variants have at least 80% sequence identity to a polypeptide selected from the group consisting of AcrIIC1, AcrIIC2, AcrIIC3, AcrIIA1, AcrIIA2, AcrIIA3, AcrIIA4, AcrIIA5, AcrIIA6, AcrVA1, AcrVA2, AcrVA3, AcrVA4, and AcrVA5.

72. The method of any one of claims 48 to 71, wherein the expression and/or activity of the Acr polypeptide and/or the CRISPR system is inducible or programmable.

73. The method of any one of claims 48 to 72, wherein the Acr polypeptide is provided to the cell by introducing into the cell a second nucleic acid molecule encoding the Acr polypeptide.

74. The method of claim 73, wherein the second nucleic acid encoding the Acr polypeptide is introduced into the cell via transient delivery.

75. The method of claim 73, wherein the Acr polypeptide is provided to the cell via stable integration of the second nucleic acid molecule into the genome of the cell.

76. The method of any one of claims 48 to 75, wherein the Acr polypeptide and/or the Cas endonuclease is operably linked to an inducible destabilization domain (DD) selected from the group consisting of a DD from the FKBP12, a DD from dihydrofolate reductase (DHFR), a DD from estrogen receptor ligand binding domain (ERLBD), and a DD from estrogen receptor (ER50).

77. The method of claim 76, wherein the stabilization of the DD is induced by addition of the stabilizing ligand rapamycin analogue compound Shield1.

78. The method of any one of claims 48 to 77, wherein the Acr polypeptide is operably linked to a proteolytic cleavage site.

79. The method of claim 78, wherein the proteolytic cleavage site can be cleaved by a protease selected from the group consisting of a tobacco etch virus (TEV) protease, a porcine teschovirus-1 2A (P2A) protease, a foot-and-mouth disease virus (FMDV) 2A (F2A) protease, an Equine Rhinitis A Virus (ERAV) 2A (E2A) protease, a Thosea asigna virus 2A (T2A) protease, a cytoplasmic polyhedrosis virus 2a (BmCPV2A) protease, a Flacherie Virus 2A (BmIFV2A) protease, thrombin, PreScission™ protease, a glutamyl endopeptidase, an Epstein-Barr virus protease, a matrix metalloproteinase 2 (MMP-2), a matrix metalloproteinase 1 (MMP-1), a membrane type 1 matrixmetalloproteinase (MT-MMP), a stromelysin 3 (or MMP-11), a matrix metalloproteinase 13 (collagenase-3), an MMP-3 (stromelysin), an MMP-7 (matrilysin), MMP-9, an NS3 protease, and a thermolysin-like MMP.

80. The method of any one of claims 48 to 79, wherein the Acr polypeptide is further linked to one or more of the following: a nuclear localization signal (NLS), a G-protein-coupled receptor (GPCR), a Gly-Ser linker, and a synthetic Notch receptor.

81. The method of any one of claims 48 to 80, wherein the Acr polypeptide and/or the one or more components of the CRISPR system are expressed episomally in the cell.

82. The method of any one of claims 48 to 80, wherein the Acr polypeptide and/or the one or more components of the CRISPR system are expressed from nucleic acids stably integrated in the genome of the cell.

83. The method of any one of claims 48 to 82, wherein the expression of the Acr polypeptide and/or the one or more components of the CRISPR system is independently under control of a constitutive promoter, a repressible promoter, or an inducible promoter.

84. The method of claim 83, wherein the constitutive promoter is selected from the group consisting of SV40 promoter, a CMV promoter, a PGK promoter, a ubiquitin C (UBC) promoter, an EF1A promoter, a CAGG promoter, and a SFFV promoter.

85. The method of claim 83, wherein the inducible promoter is selected from the group consisting of a TRE3G inducible promoter, a tetracycline-regulated promoter, a steroid-regulated promoter, a metal-regulated promoter, an estrogen receptor-regulated promoter, and a UAS inducible promoter.

86. The method of any one of claims 83 to 85, wherein the expression of the Acr polypeptide and/or the one or more components of the CRISPR system is under control of the same promoter.

87. The method of any one of claims 48 to 86, comprising:
(a) introducing into the cell a first nucleic acid molecule encoding (i) an Acr polypeptide and (ii) a sgRNA to produce an engineered cell; and
(b) transiently transfecting the engineered cell from (a) with a second nucleic acid molecule encoding a nuclease-deficient Cas polypeptide (dCas) fused to an activation domain;
wherein the Acr polypeptide inhibits the activity of the dCas-transcription domain fusion and wherein the dCas-transcription domain fusion activates the expression of the Acr polypeptide to create an incoherent feedforward loop (IFFL) gene regulation circuit.

88. The method of claim 87, wherein the first nucleic acid molecule is stably integrated into the genome of the cell.

89. The method of claim 88, wherein the first nucleic acid molecule is maintained episomally in the cell.

90. The method of any one of claims 48 to 89, wherein the cell is selected from the group consisting of a microbial cell, a fungal cell, a plant cell, and an animal cell, mammalian cell.

91. The method of claim 90, wherein the animal cell is a mammalian cell.

92. The method of claim 91, wherein the mammalian cell is a human cell.

93. The method of claim 92, wherein the human cell is a stem cell.

94. The method of claim 93, wherein the stem cell is a human-induced pluripotent stem cell (hiPSC).

95. The method of any one of claims 48 to 94, wherein the Acr polypeptide is provided to the cell concurrently with the introduction of at least one component of the CRISPR-based gene regulation system.

96. The method of any one of claims 48 to 94, wherein the Acr polypeptide is provided to the cell sequentially in relation to the introduction of the CRISPR-based gene regulation system.

97. The method of claim 96, wherein the Acr polypeptide is provided to the cell prior to the introduction of the CRISPR-based gene regulation system.

98. The method of any one of claims 48 to 97, wherein the one or more components of the CRISPR-based gene regulation system is introduced into the cell via delivery of nucleic acids encoding the one or more components or via delivery of purified ribonucleoprotein (RNP) complex.

99. A method for making a genetic circuit, comprising:
(a) providing a plurality of first nucleic acid molecules each encoding a nuclease-deficient Cas polypeptide (dCas) fused to an effector domain; and
(b) introducing each of the first nucleic acid molecules into a host cell comprising a second nucleic acid molecule to produce a plurality of engineered cells, wherein the second nucleic acid molecule encodes (i) an Acr polypeptide and (ii) a gRNA capable of directing the dCas-effector domain fusion to a target gene in the engineered cells, and
wherein the Acr polypeptide simultaneously inhibits the activity of the dCas-effector domain fusions expressed from the plurality of first nucleic acid molecules.

100. The method of claim 99, wherein the dCas polypeptideis a dCas9 polypeptide.

101. The method of claim 99, wherein the dCas polypeptide is a dCas12 polypeptide.

102. The method of any one of claims 99 to 101, wherein the dCas-effector domain fusion comprises two non-contiguous portions: an N-terminal portion (A) and a C-terminal portion (B) which, when combined, forms an active dCas-effector domain fusion.

103. The method of claim 102, wherein the Acr polypeptide inhibits the activity of the active dCas-effector domain fusion when both non-contiguous portions of dCas-effector domain fusion are present in the host cell.

104. The method of any one of claims 99 to 103, wherein the first nucleic acid molecule and/or the second nucleic acid molecule is stably integrated into the genome of the cell.

105. The method of any one of claims 99 to 103, wherein the first nucleic acid molecule and/or the second nucleic acid molecule is maintained episomally in the cell.

106. The method of any one of claims 99 to 105, wherein the target gene is an endogenous gene of the cell.

107. The method of any one of claims 99 to 105, wherein the target gene is a reporter gene selected from the group consisting of GFP, BFP, YFP, luciferase, and mCherry.

108. The method of any one of claims 99 to 107, wherein the effector domain is an activation domain.

109. The method of any one of claims 99 to 108, wherein expression of the target gene is monitored by live-cell time-lapse microscopy.

110. A genetic circuit produced by a method according to any one of claims 99 to 109.

111. A method of treating a disorder or health condition in a subject, the method comprising, the method comprising providing to a cell in the subject:
(a) an anti-CRISPR (Acr) polypeptide or nucleic acid encoding the Acr polypeptide; and
(b) a CRISPR-based system or nucleic acid encoding the system,
wherein the Acr polypeptide modulates an activity of the CRISPR-based system in said cell.


SEQUENCE LISTING (22 sequences)

SEQ ID NO: 1 - DNA, 114 bp, Artificial sequence
  Synthetic polynucleotide
  misc_feature: sgTET
gtacgttctctatcactgatagtttaagagctatgctggaaacagcatagcaagtttaaataaggctagtccgttatcaacttgaaaaagtggcaccgagtcggtgcttttttt

SEQ ID NO: 2 - DNA, 115 bp, Artificial sequence
  Synthetic polynucleotide
  misc_feature: sgSV40
gaaagtccccaggctccccagcgtttaagagctatgctggaaacagcatagcaagtttaaataaggctagtccgttatcaacttgaaaaagtggcaccgagtcggtgcttttttt

SEQ ID NO: 3 - DNA, 114 bp, Artificial sequence
  Synthetic polynucleotide
  misc_feature: sgmRFP_RR1
gaactttcagtttagcggtctgtttaagagctatgctggaaacagcatagcaagtttaaataaggctagtccgttatcaacttgaaaaagtggcaccgagtcggtgcttttttt

SEQ ID NO: 4 - DNA, 118 bp, Artificial sequence
  Synthetic polynucleotide
  misc_feature: sgCXCR4-1a
gcctctgggaggtcctgtccggctcgtttaagagctatgctggaaacagcatagcaagtttaaataaggctagtccgttatcaacttgaaaaagtggcaccgagtcggtgcttttttt

SEQ ID NO: 5 - DNA, 116 bp, Artificial sequence
  Synthetic polynucleotide
  misc_feature: sgCXCR4-3a
gcagacgcgaggaaggagggcgcgtttaagagctatgctggaaacagcatagcaagtttaaataaggctagtccgttatcaacttgaaaaagtggcaccgagtcggtgcttttttt

SEQ ID NO: 6 - DNA, 115 bp, Artificial sequence
  Synthetic polynucleotide
  misc_feature: sgCXCR4-20i
gcagaagcggccaggacattgggtttaagagctatgctggaaacagcatagcaagtttaaataaggctagtccgttatcaacttgaaaaagtggcaccgagtcggtgcttttttt

SEQ ID NO: 7 - DNA, 169 bp, Artificial sequence
  Synthetic polynucleotide
  misc_feature: sgTRP1
ggccggcatggtcccagcctcctcgctggcgccggctgggcaacaccttcgggtggcgaatgggactttccggatcaagattgtacgtagttttagagctagaaatagcaagttaaaataaggctagtccgttatcaacttgaaaaagtggcaccgagtcggtgctttt

SEQ ID NO: 8 - DNA, 152 bp, Artificial sequence
  Synthetic polynucleotide
  misc_feature: sgTETO-2x (wt + f6) MS2
acttttctctatcactgatagttttagagctagaaatagcaagttaaaataaggctagtccgttatcaacttgaaaaagtggcaccgagtcggtgcgggagcacatgaggatcacccatgtgcgactcccacagtcactggggagtcttccc

SEQ ID NO: 9 - DNA, 113 bp, Artificial sequence
  Synthetic polynucleotide
  misc_feature: sgGAL4
gagcactgtcctccgaacgtgtttaagagctaagctggaaacagcatagcaagtttaaataaggctagtccgttatcaacttgaaaaagtggcaccgagtcggtgcttttttt

SEQ ID NO: 10 - DNA, 113 bp, Artificial sequence
  Synthetic polynucleotide
  misc_feature: sg-dGFP
ggtggcggatcagaaggagggtttaagagctatgctggaaacagcatagcaagtttaaataaggctagtccgttatcaacttgaaaaagtggcaccgagtcggtgcttttttt

SEQ ID NO: 11 - DNA, 111 bp, Artificial sequence
  Synthetic polynucleotide
  misc_feature: sgPD1-ko
ggaagcggcagtcctggcgtttaagagctaagctggaaacagcatagcaagtttaaataaggctagtccgttatcaacttgaaaaagtggcaccgagtcggtgcttttttt

SEQ ID NO: 12 - DNA, 20 bp, Artificial sequence
  Synthetic polynucleotide
  misc_feature: sgRELA
gatctccacataggggccag

SEQ ID NO: 13 - DNA, 20 bp, Artificial sequence
  Synthetic polynucleotide
  misc_feature: sgCDC42BPB
gagccgcaccttggccgaca

SEQ ID NO: 14 - protein, 92 aa, Artificial sequence
  Synthetic polypeptide
  misc_feature: AcrIIC1
MAKEVFKLKPELVTYKGCGWALACIKDGEIIDLTYVRDLGIEEYDENFDGLEPEIIYYDVVASQACKEVAYRYEEMGEFTFGLCSCWEFNVM

SEQ ID NO: 15 - protein, 117 aa, Artificial sequence
  Synthetic polypeptide
  misc_feature: AcrIIC3
MAFKRAIIFTSFNGFEKVSRTEKRRLAKIINARVSIIDEYLRAKDTNASLDGQYRAFLFNDESPAMTEFLAKLKAFAESCTGISIDAWEIEESEYVRLPVERRDFLAAANGKEIFKI

SEQ ID NO: 16 - protein, 149 aa, Artificial sequence
  Synthetic polypeptide
  misc_feature: AcrIIA1
MTIKLLDEFLKKHDLTRYQLSKLTGISQNTLKDQNEKPLNKYTVSILRSLSLISGLSVSDVLFELEDIEKNSDDLAGFKHLLDKYKLSFPAQEFELYCLIKEFESANIEVLPFTFNRFENEEHVNIKKDVCKALENAITVLKEKKNELL

SEQ ID NO: 17 - protein, 123 aa, Artificial sequence
  Synthetic polypeptide
  misc_feature: AcrIIA2
MTLTRAQKKYAEAMHEFINMVDDFEESTPDFAKEVLHDSDYVVITKNEKYAVALCSLSTDECEYDTNLYLDEKLVDYSTVDVNGVTYYINIVETNDIDDLEIATDEDEMKSGNQEIILKSELK

SEQ ID NO: 18 - protein, 124 aa, Artificial sequence
  Synthetic polypeptide
  misc_feature: AcrIIA3
MTKYNKSEIMKNAWAMFNSYEWDVENFKFVSAENKTFSNCLKEAWAEEKEYVERKAKETAEAPRSEEAKAWDWACRKLNVNDLQNIDATDKVFYVVDMQKEMWTSNVWAQAIKAVELYVKLGLA

SEQ ID NO: 19 - protein, 87 aa, Artificial sequence
  Synthetic polypeptide
  misc_feature: AcrIIA4
MNINDLIREIKNKDYTVKLSGTDSNSITQLIIRVNNDGNEYVISESENESIVEKFISAFKNGWNQEYEDEEEFYNDMQTITLKSELN

SEQ ID NO: 20 - protein, 140 aa, Artificial sequence
  Synthetic polypeptide
  misc_feature: AcrIIA5 v1
MAYGKSRYNSYRKRNFSISDNQRREYAKKMKELEQAFENLDGWYLSSMKDSAYKDFGKYEIRLSNHSADNRYHDLENGRLIVNVKASKLNFVDIIENKLGKIIEKIDTLDLDKYRFINATKLERDIKCYYKGYKTKKDVI

SEQ ID NO: 21 - protein, 140 aa, Artificial sequence
  Synthetic polypeptide
  misc_feature: AcrIIA5 v2
MAYGKSRYNSYRKRSFNRSNKQRREYAQEMDRLEKAFENLDGWYLSSMKDSAYKDFGKYEIRLSNHSADNKYHDLENGRLIVNIKASKLNFVDIIENKLDKIIEKIDKLDLDKYRFINATNLEHDIKCYYKGFKTKKEVI

SEQ ID NO: 22 - protein, 183 aa, Artificial sequence
  Synthetic polypeptide
  misc_feature: AcrIIA6
MKINDDIKELILEYMSRYFKFENDFYKLPGIKFTDANWQKFKNGGTDIEKMGAARVNAMLSCLFEDFELAMIGKAQTNYYIDNSLKLNMPFYAYYDMFKKQLLINWLKNNRDDVICGTGRMYTASGNYIANAYLEVALESSRLGGGEYMLQMRFKNYSRSQEPIPSGRQNRLEWIENNLENIR